SEQUENCE LISTING (35 sequences)

SEQ ID NO: 1 - RNA, 56 nt, Artificial sequence
  Synthetic oligonucleotide
  misc_feature (1)..(12): n is a, c, g, or u
nnnnnnnnnnnnrgrurururarargrargrcrurarurgrcrurgrururururg

SEQ ID NO: 2 - protein, 1368 aa, Streptococcus pyogenes [sequence omitted]

SEQ ID NO: 3 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - RNA, 25 nt, Artificial Sequence
  Synthetic oligonucleotide
caccgggaaugcgauuuacaagcuu

SEQ ID NO: 5 - RNA, 25 nt, Artificial Sequence
  Synthetic oligonucleotide
aaacaagcuuguaaaucgcauuccc

SEQ ID NO: 6 - RNA, 25 nt, Artificial Sequence
  Synthetic oligonucleotide
caccgcuagcaauucuuagcuguca

SEQ ID NO: 7 - RNA, 25 nt, Artificial Sequence
  Synthetic oligonucleotide
aaacugacagcuaagaauugcuagc

SEQ ID NO: 8 - RNA, 25 nt, Artificial Sequence
  Synthetic oligonucleotide
caccguggcaaccauuaggaauaac

SEQ ID NO: 9 - RNA, 25 nt, Artificial Sequence
  Synthetic oligonucleotide
aaacguuauuccuaaugguugccac

SEQ ID NO: 10 - RNA, 24 nt, Artificial Sequence
  Synthetic oligonucleotide
caccgaaagugguucugucuuaaa

SEQ ID NO: 11 - RNA, 23 nt, Artificial Sequence
  Synthetic oligonucleotide
aaacuuuaagacagaaccacuuu

SEQ ID NO: 12 - RNA, 25 nt, Artificial Sequence
  Synthetic oligonucleotide
caccgaaaaucucugucuucaaaag

SEQ ID NO: 13 - RNA, 25 nt, Artificial Sequence
  Synthetic oligonucleotide
aaaccuuuugaagacagagauuuuc

SEQ ID NO: 14 - RNA, 25 nt, Artificial Sequence
  Synthetic oligonucleotide
caccgugcaugcuaaagugugugaa

SEQ ID NO: 15 - RNA, 25 nt, Artificial Sequence
  Synthetic oligonucleotide
aaacuucacacacuuuagcaugcac

SEQ ID NO: 16 - RNA, 25 nt, Artificial Sequence
  Synthetic oligonucleotide
caccgugaauauauuuuucucagcc

SEQ ID NO: 17 - RNA, 25 nt, Artificial Sequence
  Synthetic oligonucleotide
aaacggcugagaaaaauauauucac

SEQ ID NO: 18 - RNA, 25 nt, Artificial Sequence
  Synthetic oligonucleotide
caccggcauucuaccaccuggucuu

SEQ ID NO: 19 - RNA, 25 nt, Artificial Sequence
  Synthetic oligonucleotide
aaacaagaccaggugguagaaugcc

SEQ ID NO: 20 - RNA, 20 nt, Artificial Sequence
  Synthetic oligonucleotide
caagguggcgcaguuuuccc

SEQ ID NO: 21 - RNA, 20 nt, Artificial Sequence
  Synthetic oligonucleotide
gggaaaacugcgccaccuug

SEQ ID NO: 22 - RNA, 20 nt, Artificial Sequence
  Synthetic oligonucleotide
uuauguaaaaaugugugggc

SEQ ID NO: 23 - RNA, 20 nt, Artificial Sequence
  Synthetic oligonucleotide
gcccacacauuuuuacauaa

SEQ ID NO: 24 - RNA, 20 nt, Artificial Sequence
  Synthetic oligonucleotide
caucccuauaucuuaacuca

SEQ ID NO: 25 - RNA, 20 nt, Artificial Sequence
  Synthetic oligonucleotide
ugaguuaagauauagggaug

SEQ ID NO: 26 - RNA, 20 nt, Artificial Sequence
  Synthetic oligonucleotide
guaccucuucacauuguugc

SEQ ID NO: 27 - RNA, 20 nt, Artificial Sequence
  Synthetic oligonucleotide
gcaacaaugugaagagguac

SEQ ID NO: 28 - RNA, 20 nt, Artificial Sequence
  Synthetic oligonucleotide
acagcccagauaccauguug

SEQ ID NO: 29 - RNA, 20 nt, Artificial Sequence
  Synthetic oligonucleotide
caacaugguaucugggcugu

SEQ ID NO: 30 - RNA, 20 nt, Artificial Sequence
  Synthetic oligonucleotide
uuauugcuauauguaacuga

SEQ ID NO: 31 - RNA, 20 nt, Artificial Sequence
  Synthetic oligonucleotide
ucaguuacauauagcaauaa

SEQ ID NO: 32 - RNA, 20 nt, Artificial Sequence
  Synthetic oligonucleotide
cauuauugggauauccuuua

SEQ ID NO: 33 - RNA, 20 nt, Artificial Sequence
  Synthetic oligonucleotide
uaaaggauaucccaauaaug

SEQ ID NO: 34 - RNA, 20 nt, Artificial Sequence
  Synthetic oligonucleotide
aagagauuucaggcugggcg

SEQ ID NO: 35 - RNA, 20 nt, Artificial Sequence
  Synthetic oligonucleotide
cgcccagccugaaaucucuu

(19) United States
(12) Patent Application Publication
(10) Pub. No.: US 2022/0220472 A1
(43) Pub. Date: Jul. 14, 2022

(54) TARGETED THERAPY

(71) Applicant: The Regents of the University of Michigan, Ann Arbor, MI (US)

(72) Inventor: Mats Ljungman, Ann Arbor, MI (US)

(21) Appl. No.: 17/604,085

(22) PCT Filed: May 5, 2020
(86) PCT No.: PCT/US2020/031407

§ 371 Date: Oct. 15, 2021

Related U.S. Application Data
(60) Provisional application No. 62/843,893, filed on May 6, 2019.

(51) Int. Cl. C12N 15/11; A61P 35/00; C12Q 1/6886; C12N 9/22; A61K 38/46; A61K 31/5377

(52) U.S. Cl. CPC C12N 15/11; A61P 35/00; C12Q 1/6886; C12N 9/22; A61K 38/465; A61K 48/00; C12Q 2600/156; C12Q 2600/158; C12N 2310/20; C12N 2800/80; A61K 31/5377

(57) ABSTRACT

Provided herein is technology relating to treating cancer and particularly, but not exclusively, to compositions, methods, systems, and kits for selectively killing cancer cells by targeting nucleic acid rearrangement junctions (e.g., chromosome rearrangement junctions (CRJ), extrachromosomal circle junctions, etc.) with a recombinant nuclease construct.

This application claims priority to U.S. provisional patent application Ser. No. 62/843,893, filed May 6, 2019, which is incorporated herein by reference in its entirety.

### FIELD

Provided herein is technology relating to treating cancer and particularly, but not exclusively, to compositions, methods, systems, and kits for selectively killing cancer cells by targeting nucleic acid rearrangement junctions (e.g., chromosome rearrangement junctions (CRJ), extrachromosomal circle junctions, etc.) with a recombinant nuclease construct.

### BACKGROUND

Cancer is a major global health issue with tremendous physical suffering and deep economic consequences. Over 17 million new cancer cases are diagnosed and nearly 10 million people die from this disease each year. The global cancer therapeutic market has been predicted to increase from $121 billion in 2017 to $172 billion by 2022. Thus, new effective treatments are needed to improve treating this disease.

The most effective cancer therapies preferably damage and kill cancer cells relative to healthy cells. The tyrosine kinase inhibitor GLEEVEC (imatinib) is an exemplary cancer drug that specifically targets cancer cells harboring a BCR-ABL fusion protein in a certain type of leukemia without significant side effects (see, e.g., Capdeville et al. (2002) “Glivec (STI571, imatinib), a rationally developed, targeted anticancer drug” Nat Rev Drug Discov 1:493-502, incorporated herein by reference). Unfortunately, cancer research has not produced similar treatments for other cancer types caused by a single targetable fusion protein and it is unlikely that other cancers could be targeted by such an approach. Instead, current treatment regiments heavily depend on broad acting agents that are often DNA-damaging and that inflict heavy collateral side effects in patients. There is a strong need to find cancer therapies that specifically target cancer cells and that consequently minimize and/or eliminate side effects.

### SUMMARY

Nucleic acid rearrangement (e.g., chromosomal rearrangements, extrachromosomal circular DNA, etc.) have a causal role in cancer and/or are present in cancer cells (see, e.g., Rowley (2001) Nat Rev Cancer 1: 245; Koche (2020) Nature Genetics 52: 29, each of which is incorporated herein by reference). For example, most solid tumors have numerous chromosomal aberrations. It is thought that the karyotypic complexity of solid tumors is due to secondary alterations acquired through cancer evolution or progression.

Several mechanisms of nucleic acid rearrangements have been described. Generally, these rearrangements produce a rearrangement junction. In one exemplary mechanism, promoter/enhancer elements of one gene are rearranged adjacent to a proto-oncogene, thus causing altered expression of an oncogenic protein. This type of translocation is exemplified by the apposition of immunoglobulin (IG) and T-cell receptor (TCR) genes to MYC leading to activation of this oncogene in B- and T-cell malignancies, respectively (see, e.g., Rabbitts (1994) Nature 372: 143). In a second exemplary mechanism, rearrangement results in the fusion of two chromosomal regions, which may produce a fusion protein that has a new function or altered activity. The prototypic example of this translocation is the BCR-ABL gene fusion in chronic myelogenous leukemia (CML) (see, e.g., Rowley (1973) Nature 243: 290; de Klein (1982) Nature 300: 765). Importantly, this finding led to the rational development of imatinib mesylate (Gleevec), which successfully targets the BCR-ABL kinase (Deininger (2005) Blood 105: 2640). In some embodiments, the technology provided herein targets and destroys these chromosomal rearrangements to kill cancer cells.

In addition, many tumors generate extrachromosomal circular DNAs that comprise amplified oncogenes. See, e.g., Koche (2020) “Extrachromosomal circular DNA drives oncogenic genome remodeling in neuroblastoma” Nature Genetics 52: 29-34, incorporated herein by reference. In some embodiments, the technology provided herein targets and destroys these extrachromosomal circular DNAs to eliminate, reduce, and/or minimize the amplified oncogene or to eliminate, reduce, and/or minimize expression of the amplified oncogene. In some embodiments, the technology provided herein targets and destroys these extrachromosomal circular DNAs to kill cancer cells.

Provided herein is a cancer treatment technology that targets nucleic acid rearrangement junctions (e.g., chromosome rearrangement junctions (CRJ), extrachromosomal circle junctions, etc.) formed during the course of carcinogenesis and provides selective killing of cancer cells. In some embodiments, CRJ are targeted. In some embodiments, the CRJ result from a gene fusion resulting from the juxtaposition of at least a portion of a first chromosomal locus to at least a portion of a second chromosomal locus that are normally not juxtaposed (e.g., normally not near or adjacent to each other). The gene fusion need not include entire genes or exons of genes. The location where the portion of a first chromosomal locus is fused to the portion of a second chromosomal locus is the CRJ or fusion junction. These CRJs are unique for each cancer and/or tumor and are not present in normal cells. A CRJ fuses two DNA sequences that are normally distant from each other, thus creating a unique fusion junction nucleotide sequence in the CRJ DNA fusion nucleic acid. A tumor carries a set of CRJs that are identified using whole genome sequencing.

In some embodiments, an extrachromosomal circle junction is targeted. In some embodiments, the extrachromosomal circle junction results from a nucleic acid rearrangement (e.g., a nonhomologous end joining repair or replication-associated mechanism). In some embodiments, the nucleic acid rearrangement amplifies oncogenic nucleic acid sequences in the extrachromosomal circular DNA. In some embodiments, the extrachromosomal circle junction provides the selective killing of cancer cells.

Thus, according to the technology provided herein, nucleic acid rearrangement junctions (e.g., chromosome rearrangement junctions (CRJ), extrachromosomal circle junctions, etc.) are identified and targeted for cancer-specific therapy using a CRISPR/Cas9 technology. In particular, a dCas9-Fok1 fusion protein (in some embodiments, further comprising a GFP label) is used with paired guide RNAs (gRNAs) specifically designed to bind sequences adjacent to a cancer-specific nucleic acid rearrangement junction. The dCas9-Fok1 is thus targeted to a nucleic acid rearrangement junction by the gRNAs, which promotes dimerization and activation of the Fok1 endonuclease at the nucleic acid rearrangement junction and production of DNA double strand breaks in the chromosome of the cancer cells. The double strand break induces endogenous cellular surveillance pathways that may save or kill the cancer cells. Alternatively, the double strand breaks may kill cancer cells through loss of chromosome arms with essential genes.

During the development of embodiments of the technology provided herein, experiments were conducted that produced data indicating that induction of dCas9-Fok1 and 2 gRNA pairs in a doxycycline-inducible construct reduced the clonogenic survival of the colon cancer cell line HCT116 by 40-50%. Further, additional experiments were conducted that indicate that survival of cancer cells was a result of Fok1-induced double-strand DNA breaks (DSB) at the CRJs. In these experiments, the dCas9-Fok1 and gRNA were induced in the presence of a potent inhibitor of DSB repair. Data collected from these experiments indicated that cancer cells nearly completely lost their ability to form colonies when dCas9-Fok1 and gRNA pairs were expressed to induce only two double strand breaks in the presence of the DSB repair inhibitor. In control experiments, the same cancer cells expressing the dCas9-Fok1 without the gRNA pair did not show any effect of the DSB repair inhibitor. Thus, data collected during the development of embodiments of the technology described herein indicate that cancer-specific CRJs are selectively targeted with the CRISPR/dCas9-Fok1 technology described herein. Further, additional data from experiments in HCT116 cells and in vivo data using engineered HCT116 cells in xenograft mouse models indicate that inducible expression of dCas9-Fok1 and gRNA pairs reduces tumor growth rate compared to tumors not expressing the gRNA pairs or non-CRJ-targeting gRNAs.

Accordingly, in some embodiments, provided herein are methods of treating a subject having cancer or in need of a cancer treatment. For example, in some embodiments, methods comprise identifying a nucleic acid rearrangement junction (e.g., chromosome rearrangement junction (CRJ), extrachromosomal circle junction) in nucleotide sequence data obtained from a sample from said subject; and contacting a nucleic acid comprising said nucleic acid rearrangement junction with a gRNA-guided nuclease, a first gRNA, and a second gRNA. In some embodiments, methods further comprise obtaining a sample from said subject. In some embodiments, the first gRNA is complementary to a first target sequence of the nucleic acid comprising the nucleic acid rearrangement junction and the second gRNA is complementary to a second target sequence of the nucleic acid comprising the nucleic acid rearrangement junction. In some embodiments, the first target sequence and the second target sequence flank the nucleic acid rearrangement junction. In some embodiments, the first target sequence comprises the nucleic acid rearrangement junction and the second target sequence is adjacent to said nucleic acid rearrangement junction. In some embodiments of methods, methods further comprise producing or having produced the nucleotide sequence data. In some embodiments, methods further comprise sequencing nucleic acids obtained from the sample from the subject. In some embodiments, methods comprise having sequenced (e.g., by another) nucleic acids obtained from the sample from the subject. In some embodiments, the gRNA-guided nuclease is a dCas9-Fok1 protein.

In some embodiments, the gRNA-guided nuclease is a first gRNA-guided nuclease and the method further comprises contacting the nucleic acid comprising the nucleic acid rearrangement junction with a second gRNA-guided nuclease. In some embodiments, the first gRNA-guided nuclease is a dCas9-Fok1 protein and the second gRNA-guided nuclease is a dCas9-Fok1 protein. In some embodiments, the first gRNA-guided nuclease and the second gRNA-guided nuclease form a dimer In some embodiments, the dimer produces a double stranded break in the nucleic acid.

In some embodiments, methods further comprise administering an effective amount of an inhibitor of double stranded break repair to the subject. In some embodiments, methods further comprise administering an effective amount of an inhibitor of DNA-PK to the subject. In some embodiments, methods further comprise administering an effective amount of Nu7441 to said subject.

In some embodiments, the sample comprises a cancer cell. In some embodiments, the sample is obtained from, is, and/or comprises a biopsy sample from the subject.

In some embodiments, producing or having produced the nucleotide sequence data comprises use of whole genome sequencing. In some embodiments, producing or having produced the nucleotide sequence data comprises use of sequencing by synthesis or single molecule sequencing.

In some embodiments, methods comprise analyzing the nucleotide sequence data and designing the first gRNA and the second gRNA to target the nucleic acid comprising the nucleic acid rearrangement junction (e.g., chromosome rearrangement junction (CRJ), extrachromosomal circle junction). In some embodiments, methods comprise synthesizing or having synthesized the first gRNA and the second gRNA. In some embodiments, methods comprise administering the gRNA-guided nuclease or a nucleic acid encoding the gRNA-guided nuclease, the first gRNA, and the second gRNA to the subject. In some embodiments, methods comprise identifying a plurality of nucleic acid rearrangement junctions (e.g., chromosome rearrangement junctions (CRJ), extrachromosomal circle junctions, etc.) in the nucleotide sequence data. In some embodiments, methods comprise designing a specific gRNA pair targeting each nucleic acid comprising a nucleic acid rearrangement junction (e.g., chromosome rearrangement junction (CRJ), extrachromosomal circle junction). In some embodiments, methods comprise contacting each of a plurality of nucleic acids, wherein each nucleic acid comprises a nucleic acid rearrangement junction (e.g., chromosome rearrangement junction (CRJ), extrachromosomal circle junction), with a specific gRNA pair and a gRNA-guided nuclease. In some embodiments, the plurality of nucleic acid rearrangement junctions (e.g., chromosome rearrangement junctions (CRJ), extrachromosomal circle junctions, etc.) comprises 1-10, 1-20, 1-50, or 1-100 nucleic acid rearrangement junctions (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleic acid rearrangement junctions).

In some embodiments, the methods for treating a subject having cancer or in need of a cancer treatment comprise obtaining a sample from said subject; producing nucleotide sequence data from said sample; identifying a plurality of nucleic acid rearrangement junctions (e.g., chromosome rearrangement junctions (CRJ), extrachromosomal circle junctions, etc.) in said sequence data; producing a plurality of specific gRNA pairs that target nucleic acids comprising the plurality of nucleic acid rearrangement junctions (e.g., chromosome rearrangement junctions (CRJ), extrachromosomal circle junctions, etc.); and administering said plurality of said specific gRNA pairs and a gRNA-guided nuclease or a nucleic acid encoding said gRNA-guided nuclease to said subject. In some embodiments, methods further comprise administering an inhibitor of double strand break repair to the subject.

In some embodiments, the technology provides reaction mixtures. In some embodiments, the technology provides a reaction mixture comprising a gRNA-guided nuclease, a first gRNA, a second gRNA, and a nucleic acid comprising a nucleic acid rearrangement junction (e.g., chromosome rearrangement junction (CRJ), extrachromosomal circle junction). In some embodiments, the first gRNA and the second gRNA are bound to the nucleic acid comprising a nucleic acid rearrangement junction. In some embodiments, the first gRNA and the second gRNA flank the nucleic acid rearrangement junction. In some embodiments, the first gRNA binds to a sequence comprising the nucleic acid rearrangement junction and the second gRNA binds to a sequence adjacent to the nucleic acid rearrangement junction. In some embodiments, the gRNA-guided nuclease of the reaction mixture is a dCas9-Fok1. In some embodiments, reaction mixtures comprise a dimer of a gRNA-guided nuclease bound to the nucleic acid comprising the nucleic acid rearrangement junction. In some embodiments, the nucleic acid comprising a nucleic acid rearrangement junction comprises a double stranded break. In some embodiments, a first gRNA-guided nuclease of the dimer binds the first gRNA and a second gRNA-guided nuclease of the dimer binds the second gRNA-guided nuclease. Some embodiments provide an in vitro composition comprising a reaction mixture described herein. Some embodiments provide an in vivo composition comprising a reaction mixture described herein.

Some embodiments relate to a kit comprising a dCas9-Fok1 protein or a nucleic acid encoding a dCas9-Fok1 protein; and an administration means for administration.

In some embodiments, the technology provides a system for treating a subject having cancer. In some embodiment, the systems comprise a gRNA-guided nuclease or a nucleic acid encoding a gRNA-guided nuclease, a first gRNA, and a second gRNA; a nucleic acid sequencer; a software component for identifying nucleic acid rearrangement junctions (e.g., chromosome rearrangement junctions (CRJ), extrachromosomal circle junctions, etc.) in nucleic acid sequence data; a software component for designing gRNA pairs to a target nucleic acid comprising a nucleic acid rearrangement junction; and an administering component for administering said gRNA-guided nuclease or a nucleic acid encoding a gRNA-guided nuclease, said first gRNA, and said second gRNA to said subject. In some embodiments, the nucleic acid sequencer produces whole genome sequence data. In some embodiments, systems further comprise a nucleic acid synthesizer. In some embodiments, systems further comprise a sampling component to obtain a sample from the subject. In some embodiments, systems further comprise an inhibitor of double stranded break repair (e.g., an inhibitor of DNA-PK (e.g., Nu7441)).

In some embodiments, the technology provides use of a gRNA-guided nuclease, a first gRNA, and a second gRNA to treat a subject having cancer. In some embodiments, the gRNA-guided nuclease is a dCas9-Fok1 protein. In some embodiments, the technology provides use of a dCas9-Fok1 protein, a first gRNA, a second gRNA, and Nu7441 to treat a subject having cancer. In some embodiments, the first gRNA and the second gRNA provide a gRNA pair that is specific for a nucleic acid comprising a nucleic acid rearrangement junction (e.g., chromosome rearrangement junction (CRJ), extrachromosomal circle junction). In some embodiments, the technology provides use of a dCas9-Fok1 protein, a first gRNA, and a second gRNA to produce a double stranded break in a nucleic acid comprising a nucleic acid rearrangement junction in vitro. In some embodiments, the technology provides use of a dCas9-Fok1 protein, a first gRNA, and a second gRNA to produce a double stranded break in a nucleic acid comprising a nucleic acid rearrangement junction in vivo. In some embodiments of the uses, methods, systems, kits, and/or reaction mixtures described herein the nucleic acid rearrangement junction is a chromosome rearrangement junction or an extrachromosomal circle junction. In some embodiments of the uses, methods, systems, kits, and/or reaction mixtures described herein the nucleic acid rearrangement comprises a chromosomal sequence, an episomal sequence, a minicircle sequence, a mitochondrial sequence, or a chloroplast sequence. In some embodiments of the uses, methods, systems, kits, and/or reaction mixtures described herein the nucleic acid rearrangement is a DNA rearrangement junction (e.g., a chromosome rearrangement junctions (CRJ), a extrachromosomal circle junction). Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a schematic drawing showing an embodiment of the technology described herein to target a nucleic acid rearrangement junction (e.g., a chromosome rearrangement junction (CRJ), an extrachromosomal circle junction) in cancer. A nucleic acid rearrangement junction juxtaposes DNA sequences that normally are far apart to produce a fusion junction. Using gRNA specifically complementary to sequences flanking the fusion junction, two dCas9-Fok1 (optionally comprising a GFP marker) complexes are brought together to form a Fok1 dimer comprising nuclease activity that produces a DSB that is toxic to the cancer cell. Embodiments comprise augmenting toxicity using an inhibitor of DSB repair, e.g., Nu7441.

FIG. 2 shows a schematic of in vivo experiments testing dox-inducible expression of dCas9-Fok1-GFP with and without nucleic acid rearrangement junction-targeting gRNAs mice. In these experiments, HCT116 cells are injected into mice and each group is split into three treatment groups that receive ether regular water, water with Dox, or water with Dox and Nu7441.

FIG. 3 shows schematic maps of four CRJ selected for targeting in experiments conducted during the development of embodiments of the technology. In FIG. 3, the locations of the CRJ are indicated by basepair coordinates (60335540, 29523774, 130047744, and 12815669) and PAM sequences are indicated at either end of the gRNAs denoted by the shorter gray lines.

FIG. 4 is a series of fluorescence microscopy images showing that induction of dCas9-Fok1-GFP with doxycycline does not result in the formation of gH2AX foci. However, when both dCas9-Fok1-GFP and paired gRNA targeting nucleic acid rearrangement junctions were induced, approximately 2 gH2AX foci per cell were produced, indicating that both targeted nucleic acid rearrangement junctions were cut by the dimerized Fok1 endonuclease.

FIG. 5A and FIG. 5B show a series of bar plots indicating that targeting nucleic acid rearrangement junctions with dCas9-Fok1-GFP+gRNAs in HCT116 cells resulted in reduced clonogenic survival. FIG. 5A shows data collected from HCT116 cells expressing Dox-inducible dCas9-Fok1-GFP without gRNA (left-most plot) or with three different pairs of gRNAs (5ab3cd, 3ab3cd, 3abxab, as indicated). Cells were plated and treated with different concentrations of Dox for 12 days. Cells were then fixed and stained, colonies were counted, and colony numbers were reported as a percentage of control cells not treated with Dox. FIG. 5B shows data collected from HCT116 cells expressing Dox-inducible dCas9-Fok1-GFP without gRNA (top) or with three different pairs of gRNAs (5ab3cd, 3ab3cd, 3abxab, as above) in the presence of 200 nM Nu7441 during the 12-day incubations. Cells were plated and treated with different concentrations of Dox for 12 days. Cells were then fixed and stained and colonies counted and expressed as a percentage of control cells not treated with Dox. In FIG. 5A and FIG. 5B, the gRNA pairs target two CRJs on chromosome 3 (3ab and 3cd), one on chromosome 5 (5ab), and one on the X chromosome (xab).

FIG. 6A is a schematic drawing showing the chromosome locations of CRJs selected for the studies with UMUC-3 bladder cancer cells. The locations of the targeting gRNA are shown by arrows and the number 2 (targeting chromosome 2 near LRRTM4), 4 (chromosome 4), 7* (targeting chromosome 7 near IMMP2L), and 19 (targeting chromosome 19 near PSG9).

FIG. 6B is a schematic drawing showing the UMUC-3 cell lines generated with doxycycline-inducible expression of Fok1-dCas9 and gRNAs. The two controls (no-gRNA control and a control expressing non-targeting gRNA) are on the left and the 4 cell lines expressing combinations of CRJ-targeting gRNAs are on the right. Numbers (3a & 3b; 7 & 2; 7 & 4; 19 & 2; and 19 & 4) indicate the combinations of gRNA used for the experiment.

FIG. 6C is a series of bar plots showing data collected from an in vivo clonogenic survival assay in the UMUC-3 bladder cells shown schematically in FIG. 6B. The data indicated that cell fitness was decreased in cells expressing CRJ-targeting gRNAs (top row). In contrast, no toxicity was observed in the control cells. Further, treatments with the DNA-PK inhibitor Nu7441 improved the toxicity of the CRJ-targeting gRNAs (bottom row). The data are expressed as the mean and standard deviation of three biological replicates. Numbers (3a & X; 7 & 2; 7 & 4; 19 & 2; and 19 & 4) indicate the combinations of gRNA used for the experiment.

FIG. 7 is a schematic drawing showing an in vivo experiment comparing tumor growth between UMUC-3 cells expressing non-targeting gRNAs (left) and UMUC-3 cells expressing CRJ-targeting gRNAs (right). These cell lines are engineered to express luciferase for bioluminescent monitoring. Each mouse group represents 10 NODscid mice. Only the UMUC-3 cells treated with doxycycline to induce CRJ-targeting gRNA show reduced tumor growth and Nu7441 augments tumor growth inhibition.

FIG. 8 is a flowchart showing an embodiment of methods provided herein for cancer therapy.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to treating cancer and particularly, but not exclusively, to compositions, methods, systems, and kits for selectively killing cancer cells by targeting nucleic acid rearrangement junctions (e.g., chromosome rearrangement junctions (CRJ), extrachromosomal circle junctions, etc.) with a recombinant nuclease construct. Nucleic acid rearrangement (e.g., chromosome rearrangement, extrachromosomal circular DNA) is common in cancer cells and represents a biomarker of cancer because the genome of tumor cells typically harbors hundreds of such rearrangements (see, e.g., Notta et al (2016) “A renewed model of pancreatic cancer evolution based on genomic rearrangement patterns” Nature 538: 378-382; Koche (2020) “Extrachromosomal circular DNA drives oncogenic genome remodeling in neuroblastoma” Nature Genetics 52: 29-34, each of which is incorporated herein by reference).

Chromosomal rearrangements are formed early in tumorigenesis and may be formed by several mechanisms as single events or by chromothripsis (see, e.g., Notta, supra). Some characteristics of cancer cells such as, e.g., growth characteristics, nutrient requirements, etc., are selected for during cancer progression. Chromosomal rearrangements are clonal; thus, the rearrangements are present in the primary tumor and in the metastases arising from the primary tumor (see, e.g., Notta, supra). Thus, specifically targeting chromosomal rearrangements provides a technology to treat cancer systemically in a patient.

Extrachromosomal circular DNAs that comprise amplified oncogenes are often formed in tumor cells. See, e.g., Koche (2020) “Extrachromosomal circular DNA drives oncogenic genome remodeling in neuroblastoma” Nature Genetics 52: 29-34, incorporated herein by reference. Rearrangement of genomic DNA produces these extrachromosomal circular DNAs, which also are found to reintegration into the linear genome. nucleic acid rearrangement junctions are present in both the extrachromosomal circular DNA and at the chromosomal sites where circular DNA has reintegrated into the genome. In some embodiments, the technology provided herein targets and destroys these extrachromosomal circular DNAs to eliminate, reduce, and/or minimize the amplified oncogene or to eliminate, reduce, and/or minimize expression of the amplified oncogene.

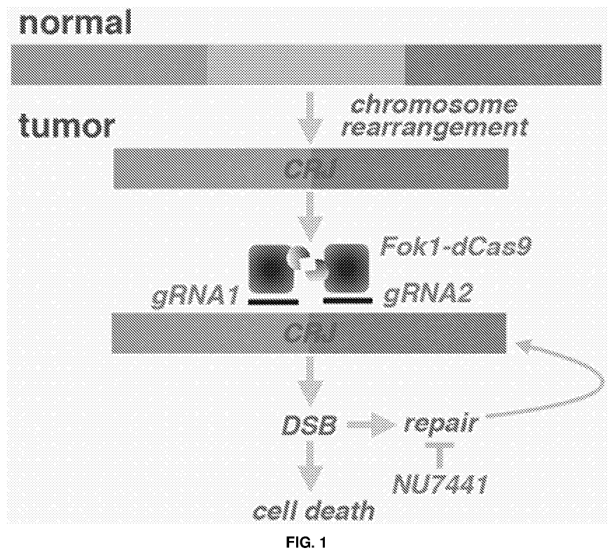

Accordingly, embodiments of the present technology relate to using a targeted nuclease (e.g., CRISPR/Cas9) technology to target this biomarker (e.g., comprising one or more nucleic acid rearrangements and/or one or more nucleic acid rearrangement junctions) of the cancer cells and specifically kill the cancer cells without affecting normal cells. In some embodiments, the technology comprises use of a protein fusion comprising dCas9 fused to the endonuclease Fok1. A dCas9-Fok1 fusion is targeted to both sides of a nucleic acid rearrangement junction by a pair of gRNAs. The two Fok1 endonucleases dimerize, thus activating the Fok1 nuclease at the nucleic acid rearrangement junction site and producing a double-stranded break (DSB) in the nucleic acid comprising the nucleic acid rearrangement junction (see, e.g., Tsai et al. (2014) “Dimeric CRISPR RNA-guided Fok1 nucleases for highly specific genome editing” Nat Biotechnol 32: 569-576, incorporated herein by reference). See, e.g., FIG. 1.

In some embodiments, the technology comprises use of a specific inhibitor of the DSB repair protein DNA-PK (e.g., Nu7441) to increase the toxicity of the generated DSB (see, e.g., FIG. 1). The technology is not limited in the inhibitor of DSB repair and is not limited in the specific inhibitor of the DSB repair protein DNA-PK. Accordingly, in some embodiments, the technology comprises use of other inhibitors of DSB repair and/or other inhibitors of DNA-PK. During the development of embodiments of the technology, experiments are conducted in which tumor cells are treated with other inhibitors of DSB repair (e.g., inhibitors of DNA-PK) and evaluated (e.g., in clinical trials) to identify one or more other inhibitors of DSB repair (e.g., inhibitors of DNA-PK) for use in combination with the dCas9-Fok1 technology to induce DSBs selectively in cancer cells. Mechanisms of DSB repair and DSB repair inhibition, including inhibitors of DSB repair (e.g., inhibitors of DNA-PK), are described, e.g., in Blackford and Jackson (2017) “ATM, ATR, and DNA-PK: The Trinity at the Heart of the DNA Damage Response” Mol Cell 66: 801-817; Pospisilova et al. (2017) “Small molecule inhibitors of DNA-PK for tumor sensitization to anticancer therapy” J Physiol Pharmacol 68: 337-344; Veuger et al. (2003) “Radiosensitization and DNA repair inhibition by the combined use of novel inhibitors of DNA-dependent protein kinase and poly(ADP-ribose) polymerase-1” Cancer Res 63: 6008-6015; and Brown et al. (2017) “Targeting DNA Repair in Cancer: Beyond PARP Inhibitors” Cancer Discov 7: 20-37, each of which is incorporated herein by reference.

Experiments conducted during the development of embodiments of the technology provided herein produced data using the colon cancer cell line HCT116 indicating that inducing expression of dCas9-Fok1 and 2 pairs of gRNAs targeting cancer-specific nucleic acid rearrangement junctions (e.g., CRJ) reduced the fitness (e.g., survival) of these cells. Furthermore, the data indicated that combining this treatment with a non-toxic concentration of an inhibitor of DSB repair (e.g., an inhibitor of DNA-PK (e.g., Nu7441)) produced improved toxicity and killing. Accordingly, the technology provided herein effectively kills cancer cells by specifically targeting nucleic acid rearrangement junctions. Further, experiments are conducted to evaluate the technology in cell lines and in vivo to reduce the fitness (e.g., survival) of cancer cells in tumors.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the term “gRNA-targeted nuclease” refers to a protein (e.g., a fusion protein) comprising 1) a nuclease domain and/or a protein domain having nuclease activity that produces double-stranded breaks in a nucleic acid; and 2) a gRNA-binding domain that directs the gRNA-targeted nuclease to target a nucleic acid with sequence specificity. In some embodiments, the gRNA-targeted nuclease comprises: 1) a Cas9 or a similar protein (e.g., a Cpf1 or other Cas9-like protein or Cas9 homolog as described herein) having a gRNA binding and targeting activity similar to a Cas9 but with minimized and/or eliminated nuclease activity (e.g., a “dead Cas9” or similar “dead” Cpf1 or other “dead” Cas9-like protein or “dead” Cas9 homolog as described herein) that is fused to: 2) a Fok1 nuclease

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982), incorporated herein by reference). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 2002, 41(14), 4503-4510, incorporated herein by reference) and U.S. Pat. No. 5,034,506, incorporated herein by reference), locked nucleic acid (LNA; see Wahlestedt et al., Proc. Natl. Acad. Sci. U.S.A., 2000, 97, 5633-5638, incorporated herein by reference), cyclohexenyl nucleic acids (see Wang, J. Am. Chem. Soc., 2000, 122, 8595-8602, incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

Furthermore, the terms “nucleic acid”, “polynucleotide”, “nucleotide sequence”, and “oligonucleotide” are used interchangeably. They refer to a polymeric form of nucleotides of any length, either deoxyribonucleotides or ribonucleotides, or analogs thereof. Polynucleotides may have any three dimensional structure and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996, each of which is incorporated herein by reference. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner, herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152, each of which is incorporated herein by reference). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the term “% sequence identity” refers to the percentage of nucleotides or nucleotide analogs in a nucleic acid sequence that is identical with the corresponding nucleotides in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “homology” and “homologous” refers to a degree of identity. There may be partial homology or complete homology. A partially homologous sequence is one that is less than 100% identical to another sequence.

The term “sequence variation” as used herein refers to a difference or multiple differences in nucleic acid sequence between two nucleic acids. For example, a wild-type structural gene and a mutant form of this wild-type structural gene may vary in sequence by the presence of one or more single base substitutions or by deletions and/or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

As used herein, the terms “complementary”, “hybridizable”, or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-S′.” Complementarity may be “partial,” in which only some of the nucleic acid bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., nucleotides that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

It is understood in the art that the sequence of a polynucleotide need not be 100% complementary to that of its target nucleic acid to be hybridizable or specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, a nucleic acid in which 18 of 20 nucleotides of the nucleic acid are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular segments of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656, each of which is incorporated herein by reference) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489, incorporated herein by reference).

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

“Mismatch” means a nucleobase of a first nucleic acid that is not capable of pairing with a nucleobase at a corresponding position of a second nucleic acid.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960), each of which is incorporated herein by reference, have been followed by the refinement of this process into an essential tool of modern biology. For example, hybridization and washing conditions are now well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001), each of which is incorporated herein by reference. The conditions of temperature and ionic strength determine the “stringency” of the hybridization.

As used herein, the term “Tm” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation: Tₘ=81.5+0.41*(% G+C), when a nucleic acid is in aqueous solution at 1 M NaCl (see e.g., Anderson and Young, Quantitative Filter Hybridization, in Nucleic Acid Hybridization (1985). Other references (e.g., Allawi and SantaLucia, Biochemistry 36: 10581-94 (1997) include more sophisticated computations which account for structural, environmental, and sequence characteristics to calculate Tₘ. For example, in some embodiments these computations provide an improved estimate of Tₘ for short nucleic acid probes and targets (e.g., as used in the examples).

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) comprises a “double-stranded nucleic acid”. For example, triplex structures are considered to be “double-stranded”. In some embodiments, any base-paired nucleic acid is a “double-stranded nucleic acid”.

As used herein, the term “genomic locus” or “locus” (plural “loci”) is the specific location of a gene or DNA sequence on a chromosome.

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this invention it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “functional derivative” of a polypeptide is a compound having a qualitative biological property in common with said polypeptide. “Functional derivatives” include, but are not limited to, fragments of polypeptide and derivatives of a polypeptide and its fragments, provided that they have a biological activity in common with a corresponding polypeptide. The term “derivative” encompasses both amino acid sequence variants of polypeptide, covalent modifications, and fusions thereof. A “fusion” polypeptide is a polypeptide comprising a polypeptide or portion (e.g., one or more domains) thereof fused or bonded to another heterologous polypeptide.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, the term “nuclease-deficient” refers to a protein comprising reduced nuclease activity, minimized nuclease activity (e.g., a nickase), undetectable nuclease activity, and/or having no nuclease activity, e.g., as a result of amino acid substitutions that reduce, minimize, and/or eliminate the nuclease activity of a protein. In some embodiments, a nuclease-deficient protein is described as a “dead” protein.

The term “oligonucleotide” as used herein is defined as a molecule comprising two or more deoxyribonucleotides or ribonucleotides, preferably at least 5 nucleotides, in some embodiments at least about 10 to 15 nucleotides and in some embodiments at least about 15 to 50 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides). The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide (e.g., a dCas9 protein or domain or similar protein or domain)) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein binding protein). In the case of a protein domain-binding protein, it can bind to itself (to form homodimers, homotrimers, etc.) and/or it can bind to one or more molecules of a different protein or proteins.

As used herein, the term “ribonucleoprotein”, abbreviated “RNP” refers to a multimolecular complex comprising a polypeptide (e.g., gRNA-targeted nuclease (e.g., a Cas9, a dCas9, a dCas9-Fok1 fusion protein, or a protein having an activity similar to a Cas9, a dCas9, a dCas9-Fok1 fusion protein (e.g., a Cpf1, Cpf1-Fok1 fusion protein, or other Cas9-like protein, Cas9 homolog, and/or Fok1 fusion thereof))) and a ribonucleic acid (e.g., a gRNA (e.g., sgRNA, a dgRNA)). In some embodiments, the polypeptide and ribonucleic acid are bound by a non-covalent interaction.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine/isoleucine, phenylalanine-tyrosine, lysine-arginine, alanine-valine, and asparagine-glutamine.

“Recombinant,” as used herein, means that a particular nucleic acid (DNA or RNA) is the product of various combinations of cloning, restriction, polymerase chain reaction (PCR), and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms). Alternatively, DNA sequences encoding RNA (e.g., DNA-targeting RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, e.g., an “insert”, may be attached so as to bring about the replication of the attached segment in a cell.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam and Labhasetwar (2012), Advanced Drug Delivery Reviews, 64 (supplement): 61-71, incorporated herein by reference). The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995, incorporated herein by reference.

A “target nucleic acid” (e.g., a “target DNA”) as used herein is a polynucleotide (nucleic acid, gene, chromosome, genome, etc.) that comprises a “target site” or “target sequence.” The terms “target site” or “target sequence” are used interchangeably herein to refer to a nucleic acid sequence present in a target DNA to which a DNA-targeting segment of a DNA-targeting RNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand”.

The RNA molecule that binds to the polypeptide in the RNP and targets the polypeptide to a specific location within the target DNA is referred to herein as the “DNA targeting RNA” or “DNA-targeting RNA polynucleotide” (also referred to herein as a “guide RNA” or “gRNA”). A DNA-targeting RNA comprises two segments, a “DNA-targeting segment” and a “protein-binding segment.” In some embodiments, the gRNA comprises two RNAs (e.g., a dgRNA, e.g., a crRNA and a tracrRNA) and in some embodiments the gRNA comprises one RNA (e.g., a sgRNA).

By “segment” it is meant a segment or section or portion or region of a molecule, e.g., a contiguous segment of nucleotides in an RNA, DNA, or protein. A segment can also mean a segment or section or portion or region of a complex such that a segment may comprise regions of more than one molecule. For example, in some embodiments the protein-binding segment (described below) of a DNA targeting RNA is one RNA molecule and the protein-binding segment therefore comprises a region of that RNA molecule. In other cases, the protein-binding segment (described below) of a DNA-targeting RNA comprises two separate molecules that are hybridized along a region of complementarity. As an illustrative, non-limiting example, a protein-binding segment of a DNA targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA (the complementary strand of the target DNA). The protein-binding segment (or “protein-binding sequence”) interacts with a polypeptide of the RNP. The protein-binding segment of a DNA-targeting RNA comprises two complementary segments of nucleotides that hybridize to one another to form a double stranded RNA duplex (dsRNA duplex).

A DNA-targeting RNA and a polypeptide form a RNP complex (e.g., bind via non-covalent interactions). The DNA-targeting RNA provides target specificity to the RNP complex by comprising a nucleotide sequence that is complementary to a sequence of a target DNA. The polypeptide of the RNP complex provides site-specific binding and, in some embodiments, a nuclease activity (e.g., for producing a double-strand break in a chromosome). In other words, the polypeptide of the RNP is guided to a target DNA sequence (e.g., a target sequence in a chromosomal nucleic acid; a target sequence in an extrachromosomal nucleic acid (e.g., an episomal nucleic acid, a minicircle, etc.); a target sequence in a mitochondrial nucleic acid; a target sequence in a chloroplast nucleic acid; a target sequence in a plasmid; etc.) by virtue of its association with the protein-binding segment of the DNA-targeting RNA.

In some embodiments, a DNA-targeting RNA comprises two separate RNA molecules (e.g., two RNA polynucleotides, e.g., an “activator-RNA” and a “targeter-RNA”) and is referred to herein as a “double-molecule DNA-targeting RNA” or a “two-molecule DNA-targeting RNA” or a “double guide RNA” or a “dgRNA”. In other embodiments, the DNA-targeting RNA is a single RNA molecule (e.g., a single RNA polynucleotide) and is referred to herein as a “single-molecule DNA-targeting RNA,” a “single guide RNA,” or an “sgRNA.” The term “DNA-targeting RNA” or “guide RNA” or “gRNA” is inclusive, referring both to double-molecule DNA-targeting RNAs (dgRNAs) and to single-molecule DNA-targeting RNAs (sgRNAs).

An exemplary two-molecule DNA-targeting RNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the DNA-targeting segment (single stranded) of the DNA-targeting RNA and a region (“duplex-forming segment”) that forms one half of the dsRNA duplex of the protein-binding segment of the DNA-targeting RNA. A corresponding tracrRNA-like molecule (activator-RNA) comprises a region (duplex-forming segment) that forms the other half of the dsRNA duplex of the protein-binding segment of the DNA-targeting RNA. In other words, a portion of the crRNA-like molecule is complementary to and hybridizes with a portion of a tracrRNA-like molecule to form the dsRNA duplex of the protein-binding domain of the DNA-targeting RNA. As such, each crRNA-like molecule can be said to have a corresponding tracrRNA-like molecule. The crRNA-like molecule additionally provides the single stranded DNA-targeting segment.

Thus, a crRNA-like molecule (e.g., a crRNA) and a tracrRNA-like molecule (e.g., a tracrRNA) hybridize (as a corresponding pair) to form a DNA-targeting RNA. The exact sequence of a given crRNA or tracrRNA molecule is characteristic of the species in which the RNA molecules are found. Various crRNAs and tracrRNAs are known in the art. A subject double molecule DNA-targeting RNA (dgRNA) can comprise any corresponding crRNA and tracrRNA pair. A subject double-molecule DNA-targeting RNA (sgRNA) can comprise any corresponding crRNA and tracrRNA pair.

The term “activator-RNA” is used herein to mean a tracrRNA-like molecule of a double molecule DNA-targeting RNA (e.g., a tracrRNA). The term “targeter-RNA” is used herein to mean a crRNA-like molecule of a double-molecule DNA-targeting RNA (e.g., a crRNA). The term “duplex-forming segment” is used herein to mean the segment of an activator-RNA or a targeter-RNA that contributes to the formation of the dsRNA duplex by hybridizing to a segment of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the DNA-targeting segment of the DNA-targeting RNA. Therefore, a subject double-molecule DNA-targeting RNA can be comprised of any corresponding activator-RNA and targeter-RNA pair.

As used herein, “CRISPR system” refers collectively to transcripts and other elements involved in the expression of and/or directing the activity of CRISPR-associated (“Cas”) genes, including sequences encoding a Cas gene, dCas gene, Cas homolog, and/or Cpf1 gene; a tracr (trans-activating CRISPR) sequence (e.g., tracrRNA or an active partial tracrRNA); a cr (CRISPR) sequence (e.g., crRNA or an active partial crRNA); and/or other sequences and transcripts from a CRISPR locus. In some embodiments of the technology, the terms “guide sequence” and “guide RNA” (gRNA) are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR RNP complex (e.g., in vitro or in vivo) and direct it to the site of a target sequence in a cell (e.g., after introduction of the RNP).

As used herein, the term “CRISPR activity” refers to an activity associated with a CRISPR system. Examples of such activities are sequence-specific binding, double-stranded nuclease activity, nickase activity, transcriptional activation, transcriptional repression, nucleic acid methylation, nucleic acid demethylation, and recombinase.

As used herein, the terms “subject” and “patient” refer to any organisms including plants, microorganisms, and animals (e.g., mammals such as dogs, cats, livestock, and humans).

The terms “treatment”, “treating”, and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, e.g., arresting its development; or (c) relieving the disease, e.g., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and In some embodiments after the symptomatic stage of the disease

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

The term “label” as used herein refers to any atom or molecule that can be used to provide a detectable (preferably quantifiable) effect, and that can be attached to a nucleic acid or protein. Labels include, but are not limited to, dyes (e.g., fluorescent dyes or moities); radiolabels such as ³²P; binding moieties such as biotin; haptens such as digoxgenin; luminogenic, phosphorescent, or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

As used herein, “moiety” refers to one of two or more parts into which something may be divided, such as, for example, the various parts of an oligonucleotide, a molecule, a chemical group, a domain, a probe, etc.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the technology encompass a variety of conditions wherein cell division is deregulated. Exemplary cell proliferative disorders include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue.

A cell proliferative disorder includes a precancer or a precancerous condition. A cell proliferative disorder includes cancer. In some embodiments, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers. In some embodiments, cancer cells or precancerous cells are identified by the presence of one or more nucleic acid rearrangements that produce one or more nucleic acid rearrangement junctions (e.g., chromosome rearrangement junction (CRJ), extrachromosomal circle junction).

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute lymphocytic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

As used herein, a “normal cell” is a cell that cannot be classified as part of a “cell proliferative disorder”. A normal cell lacks unregulated or abnormal growth, or both, that can lead to the development of an unwanted condition or disease. In some embodiments, a normal cell possesses normally functioning cell cycle checkpoint control mechanisms.

As used herein, “contacting a cell” refers to a condition in which a compound or other composition of matter is in direct contact with a cell or is close enough to induce a desired biological effect in a cell.

As used herein, the term “nucleic acid rearrangement junction” refers to a junction formed from the juxtaposition of at least a portion of a first nucleic acid to at least a portion of a second nucleic acid that results from a nucleic acid rearrangement, e.g., in a cancer cell. The nucleic acid rearrangement may be a DNA rearrangement or an RNA rearrangement and, accordingly, the DNA rearrangement comprises a DNA rearrangement junction and an RNA rearrangement comprises an RNA rearrangement junction. In some embodiments, the nucleic acid rearrangement produces an abnormal dosage of gene(s) located within the rearranged genomic fragments. The nucleic acid rearrangement junction may comprise and/or be formed in, e.g., a chromosomal sequence, an episomal sequence, a minicircle sequence, a mitochondrial sequence, a chloroplast sequence, etc. Exemplary nucleic acid rearrangements that produce nucleic acid rearrangement junctions include chromosomal rearrangements (e.g., interchromosomal rearrangements and intrachromosomal rearrangements), extrachromosomal circular DNA, gene duplication, gene amplification, low copy repeats (LCRs), repeat gene clusters, and segmental duplications. nucleic acid rearrangement junctions may be formed from any nucleic acid in a cell (e.g., genomic DNA), e.g., portions of genes or from non-coding portions of the genome. See, e.g., Lupski (1998) “Genomic disorders: structural features of the genome can lead to nucleic acid rearrangements and human disease traits” Trends Genet 14: 417-22; Stankiewicz (2002) “Genome architecture, rearrangements and genomic disorders” Trends Genet 18: 74-82; and Carvalho (2016) “Mechanisms underlying structural variant formation in genomic disorders” Nat Rev Genet 17: 224-38, each of which is incorporated herein by reference.

### Description

Although the disclosure herein refers to certain illustrated embodiments, it is to be understood that these embodiments are presented by way of example and not by way of limitation.

### CRISPR/Cas9

CRISPR/Cas9 technology has revolutionized scientific research and has begun to revolutionize clinical practices (see, e.g., Barrangou and Doudna (2016) “Applications of CRISPR technologies in research and beyond. Nat Biotechnol 34: 933-941; Tsai and Joung (2016) “Defining and improving the genome-wide specificities of CRISPR-Cas9 nucleases” Nat Rev Genet 17: 300-312, each of which is incorporated herein by reference). CRISPR/Cas9 and related technologies are based on targeting specific sequences in a genome using gRNAs complementary to sequences of interest. The CRISPR/Cas9 system is commonly used to inactivate and/or delete critical sequences of a gene through endonucleolytic cleavage followed by mutagenic repair by the host cell (see, e.g., Tsai and Joung, supra). While the original CRISPR/Cas9 technology has found widespread use, the technology also includes CRISPR/Cas9-like systems discovered in archaea and compact CRISPR/CasX, CRISPR/CasY, and Cas13 systems discovered in bacteria (see, e.g., Cloney (2017) “Metagenomics: Uncultivated microbes reveal new CRISPR-Cas systems” Nat Rev Genet 18: 146; Burstein et al (2017) “New CRISPR-Cas systems from uncultivated microbes” Nature 542: 237-241; Cox et al. (2017) “RNA editing with CRISPR-Cas13” Science 358: 1019-1027, each of which is incorporated herein by reference). These Cas9-like proteins and system greatly increase the versatility of this targeting approach (see, e.g., Cloney, Burstein et al., Cox et al., supra, and Abudayyeh et al (2017) “RNA targeting with CRISPR-Cas13” Nature 550: 280-284, each of which is incorporated herein by reference).

Further, alterations in Cas9 that minimize and/or eliminate block its catalytic activity have been engineered (e.g., producing “dead” proteins such as “dCas9”) and used to site-specifically inhibit (or activate) particular genes (see, e.g., Qi et al. (2013) “Repurposing CRISPR as an RNA-guided platform for sequence-specific control of gene expression” Cell 152: 1173-1183; and Gilbert et al (2013) “CRISPR-mediated modular RNA-guided regulation of transcription in eukaryotes. Cell 154: “442-451”, each of which is incorporated herein by reference) and to bring together two halves of proteins at particular genomic sequences (see, e.g., Gilbert, supra). In some embodiments, the technology provided herein relates to the use of a “dead” CRISPR protein (e.g., a Cas9, Cas9 homolog, and/or other gRNA-guided protein) to target nucleic acid rearrangement junctions (e.g., chromosome rearrangement junctions (CRJ), extrachromosomal circle junctions, etc.) and produce double-strand breaks in chromosomal DNA.

Also, a variety of Cas9 variants have been generated that possess different requirements for the PAM sequence, a sequence which needs to be located next to the sequence recognized by the guide RNA, thus dramatically increasing the variety of genomic sequences that can be targeted (see, e.g., Kleinstiver et al. (2015) “Broadening the targeting range of Staphylococcus aureus CRISPR-Cas9 by modifying PAM recognition” Nat Biotechnol 33: 1293-1298; Kleinstiver et al (2015) “Engineered CRISPR-Cas9 nucleases with altered PAM specificities” Nature 523: 481-485; Hirano et al. (2016) “Structural Basis for the Altered PAM Specificities of Engineered CRISPR-Cas9” Mol Cell 61: 886-894; and Walton (2020) “Unconstrained genome targeting with near-PAMless engineered CRISPR-Cas9 variants” Science 368: 290-96, each of which is incorporated herein by reference). And, recent refinement of Cas9 sequences has dramatically increased the specificity of targeting (see, e.g., Slaymaker et al. (2016) “Rationally engineered Cas9 nucleases with improved specificity” Science 351: 84-88; and Kleinstiver et al. (2016) “High-fidelity CRISPR-Cas9 nucleases with no detectable genome-wide off-target effects” Nature 529: 490-495, each of which is incorporated herein by reference). In some embodiments, the technology comprises a “dead” Cas9 protein as known in the art to have an altered PAM sequence.

In some embodiments, the technology comprises use of a ribonucleoprotein (RNP) comprising a gRNA-targeted nuclease (e.g., a dCas9-Fok1 fusion) that produces a double-stranded break (DSB) in DNA at a nucleic acid rearrangement junction (e.g., chromosome rearrangement junction (CRJ), extrachromosomal circle junction). In some embodiments, the technology comprises use of a RNP complex comprising a dCas9, dCas9-like protein, and/or a domain of a dCas9 or dCas9-like protein and an RNA (e.g., e.g., a gRNA (e.g., a subject DNA-targeting RNA, an activator-RNA and a targeter-RNA, a crRNA and a tracrRNA; a dgRNA; a sgRNA)). In some embodiments, the protein is a Cas9 or Cas9-like protein having minimized and/or eliminated nuclease activity (“dCas9”) fused to a Fok1 nuclease domain (“dCas9-Fok1” or “dCas9-Fok1 protein fusion”) as described herein. Thus, in some embodiments the technology comprises use of a ribonucleoprotein (RNP) complex comprising a dCas9 or dCas9-like protein fused to a Fok1 domain (“dCas9-Fok1” or “dCas9-Fok1 protein fusion”) as described herein and an RNA (e.g., e.g., a gRNA (e.g., a subject DNA-targeting RNA, an activator-RNA and a targeter-RNA, a crRNA and a tracrRNA; a dgRNA; a sgRNA)).

The RNA provides target specificity to the RNP complex by comprising a nucleotide sequence that is complementary to a sequence of a target DNA (e.g., at or near a nucleic acid rearrangement junction (e.g., chromosome rearrangement junction (CRJ), extrachromosomal circle junction)). The polypeptide of the complex provides binding and nuclease activity. In other words, the polypeptide is guided to a DNA sequence (e.g. a chromosomal sequence (e.g., at or near a nucleic acid rearrangement junction (e.g., chromosome rearrangement junction (CRJ), extrachromosomal circle junction)) or an extrachromosomal sequence (e.g., an episomal sequence, a minicircle sequence, a mitochondrial sequence, a chloroplast sequence, etc.)) by virtue of its association with at least the protein-binding segment of the DNA-targeting RNA.

While various CRISPR/Cas systems have been used extensively for genome editing in cells of various types and species, recombinant and engineered nucleic acid-binding proteins such as Cas9 and Cas9-like proteins find use in the present technology to provide a gRNA-targeted nuclease (e.g., a dCas9-Fok1 fusion protein). Embodiments of the technology provide an RNP comprising a polypeptide, e.g., a dCas9-Fok1 fusion protein or a related or similar protein. The Cas9 protein was discovered as a component of the bacterial adaptive immune system (see, e.g., Barrangou et al. (2007) “CRISPR provides acquired resistance against viruses in prokaryotes” Science 315: 1709-1712, incorporated herein by reference). Cas9 is an RNA-guided endonuclease that targets and destroys foreign DNA in bacteria using RNA:DNA base-pairing between a guide RNA (gRNA) and foreign DNA to provide sequence specificity. Recently, Cas9/gRNA complexes (e.g., a Cas9/gRNA RNP) have found use in genome editing (see, e.g., Doudna et al. (2014) “The new frontier of genome engineering with CRISPR-Cas9” Science 346: 6213, incorporated herein by reference).

Accordingly, some Cas9/RNA RNP complexes comprise two RNA molecules: (1) a CRISPR RNA (crRNA), possessing a nucleotide sequence complementary to the target nucleotide sequence; and (2) a trans-activating crRNA (tracrRNA). In this mode, Cas9 functions as an RNA-guided nuclease that uses both the crRNA and tracrRNA to recognize and cleave a target sequence. Recently, a single chimeric guide RNA (sgRNA) mimicking the structure of the annealed crRNA/tracrRNA has become more widely used than crRNA/tracrRNA because the gRNA approach provides a simplified system with only two components (e.g., the dCas9 or dCas9-Fok1 fusion and the gRNA). Thus, sequence-specific binding of the RNP to a nucleic acid can be guided by a dual-RNA complex (e.g., a “dgRNA”), e.g., comprising a crRNA and a tracrRNA in two separate RNAs or by a chimeric single-guide RNA (e.g., a “sgRNA”) comprising a crRNA and a tracrRNA in a single RNA. (see, e.g., Jinek et al. (2012) “A Programmable Dual-RNA-Guided DNA Endonuclease in Adaptive Bacterial Immunity” Science 337:816-821, incorporated herein by reference).

As used herein, the targeting region of a crRNA (2-RNA dgRNA system) or a sgRNA (single guide system) is referred to as the “guide RNA” (gRNA). In some embodiments, the gRNA comprises, consists of, or essentially consists of 10 to 50 bases, e.g., 15 to 40 bases, e.g., 15 to 30 bases, e.g., 15 to 25 bases (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases). Methods are known in the art for determining the length of the gRNA that provides the most efficient target recognition for a Cas9. See, e.g., Lee et al. (2016) “The Neisseria meningitidis CRISPR-Cas9 System Enables Specific Genome Editing in Mammalian Cells” Molecular Therapy 24: 645 (2016), incorporated herein by reference.

Accordingly, in some embodiments, the gRNA is a short synthetic RNA comprising a “scaffold sequence” (protein-binding segment) for protein binding (e.g., for Cas9, dCas9, or dCas9-Fok1 binding) and a user-defined “DNA-targeting sequence” (DNA-targeting segment) that is approximately 20-nucleotides long and is complementary to the target site of the target nucleic acid.

In some embodiments, DNA targeting specificity is determined by two factors: 1) a DNA sequence matching the gRNA targeting sequence and a protospacer adjacent motif (PAM) directly downstream of the target sequence. Some Cas9/gRNA complexes recognize a DNA sequence comprising a protospacer adjacent motif (PAM) sequence and an adjacent sequence comprising approximately 20 bases complementary to the gRNA. Canonical PAM sequences are NGG or NAG for Cas9 from Streptococcus pyogenes and NNNNGATT for the Cas9 from Neisseria meningitidis. In some embodiments, the technology comprises use of a Cas9 having an expanded PAM recognition (e.g., an xCas9 protein). Following DNA recognition by hybridization of the gRNA to the DNA target sequence, Cas9 cleaves the DNA sequence via an intrinsic nuclease activity. For genome editing and other purposes, the CRISPR/Cas system from S. pyogenes has been used most often. Using this system, one can target a given target nucleic acid (e.g., for editing or other manipulation) by designing a gRNA comprising a nucleotide sequence complementary to a DNA sequence (e.g., a DNA sequence comprising approximately 20 nucleotides) that is 5′-adjacent to the PAM. Methods are known in the art for determining a PAM sequence that provides efficient target recognition for a Cas9 (and thus for a gRNA-guided nuclease (e.g., dCas9-Fok1 fusion protein)). See, e.g., Zhang et al. (2013) “Processing-independent CRISPR RNAs limit natural transformation in Neisseria meningitidis” Molecular Cell 50: 488-503, incorporated herein by reference; Lee et al., supra, incorporated herein by reference.

In some exemplary embodiments, the crRNA comprises a sequence according to SEQ ID NO: 1:

NNNNNNNNNNNNrGrUrUrUrArArGrArGrCrUrArUrGrCrUrGrUrUrUrUrG

where the “NNNNNNNNNNNN” represents the DNA-targeting sequence that is complementary to the target sequence (e.g., of a nucleic acid to be subject to editing (e.g., knockin)). In some embodiments, the 5′ end of the crRNA comprises a detectable label, e.g., a dye, e.g., a fluorescent dye.

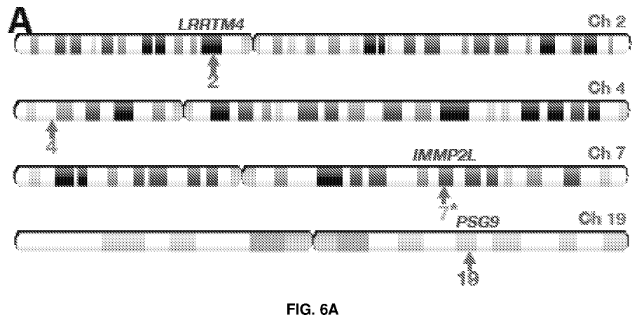

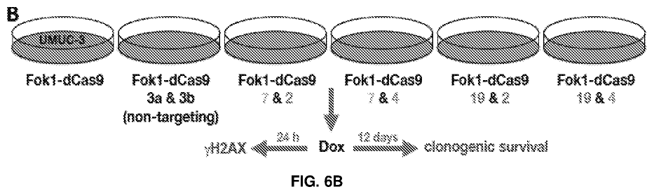

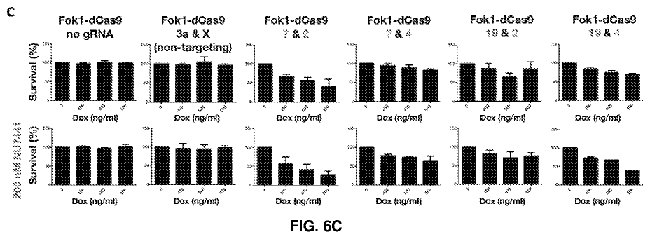

In some embodiments, the tracrRNA comprises a sequence of a naturally occurring tracrRNA, e.g., a provided by FIGS. 6, 35, and 37, and by SEQ ID NOs: 267-272 and 431-562 of U.S. Pat. App. Pub. No. 20170051312, incorporated herein by reference.

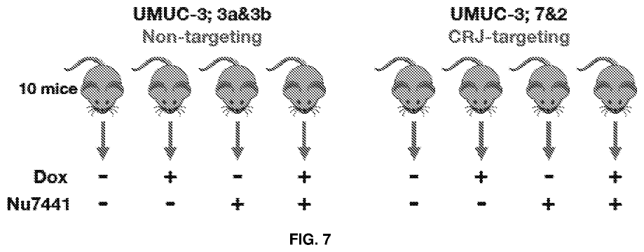

In some embodiments, the crRNA comprises a sequence that hybridizes to a tracrRNA to form a duplex structure, e.g., a sequence provided by FIG. 7 and SEQ ID NOs: 563-679 of U.S. Pat. App. Pub. No. 20170051312, incorporated herein by reference. In some embodiments, a crRNA comprises a sequence provided by FIG. 37 of U.S. Pat. App. Pub. No. 20170051312, incorporated herein by reference. In some embodiments, the duplex-forming segment of the crRNA is at least about 60% identical to one of the tracrRNA molecules set forth in SEQ ID NOs: 431-679 of U.S. Pat. App. Pub. No. 20170051312, incorporated herein by reference, or a complement thereof.

Thus, in some embodiments, exemplary (but not limiting) nucleotide sequences that are included in a dgRNA system include either of the sequences set forth in U.S. Pat. App. Pub. No. 20170051312, incorporated herein by reference, as SEQ ID NOs: 431-562, or complements thereof pairing with any sequences set forth in U.S. Pat. App. Pub. No. 20170051312, incorporated herein by reference, SEQ ID NOs: 563-679, or complements thereof that can hybridize to form a protein binding segment.

In some embodiments, a single-molecule gRNA (e.g., a sgRNA) comprises two complementary stretches of nucleotides that hybridize to form a dsRNA duplex. In some embodiments, the sgRNA (or a DNA encoding the sgRNA) is at least about 60% identical to one of the tracrRNA molecules set forth in U.S. Pat. App. Pub. No. 20170051312, incorporated herein by reference, as SEQ ID NOs: 431-562, or a complement thereof, over at least 8 contiguous nucleotides. In some embodiments, the sgRNA (or a DNA encoding the sgRNA) is at least about 60% identical to one of the tracrRNA molecules set forth in U.S. Pat. App. Pub. No. 20170051312, incorporated herein by reference, as SEQ ID NOs: 563-679, or a complement thereof, over at least 8 contiguous nucleotides. Appropriate naturally occurring pairs of crRNAs and tracrRNAs can be routinely determined by taking into account the species name and base-pairing (for the dsRNA duplex of the protein-binding domain) when determining appropriate cognate pairs.

In some embodiments, the technology provides a gRNA-targeted nuclease fusion protein that comprises Fok1 and dCas9 (e.g., dCas9-Fok1). In some embodiments, a dCas9-Fok1/gRNA complex binds to a target nucleic acid with a sequence specificity provided by the gRNA to produce a double strand break in the nucleic acid. In some embodiments, the dCas9-Fok1/gRNA RNP binds to the target nucleic acid with sequence specificity. In some embodiments, the dCas9-Fok1 fusion is a protein provided by U.S. Pat. App. Pub. No. 2015/0071899, incorporated herein by reference. In some embodiments, a Cas9-Fok1 fusion is modified to inhibit, minimize, and/or eliminate the nuclease activity of the Cas9 to produce a dCas9-Fok1 fusion as described herein (e.g., comprising one or more amino acid substitutions as described herein for dCas9).

Furthermore, while the Cas9/gRNA system initially targeted sequences adjacent to a PAM, in some embodiments the dCas9-Fok1/gRNA system as used herein has been engineered to target any nucleotide sequence for binding (e.g., the technologies described herein are PAM-independent). Also, Cas9 orthologs encoded by compact genes (e.g., Cas9 from Staphylococcus aureus) are known (see, e.g., Ran et al. (2015) “In vivo genome editing using Staphylococcus aureus Cas9” Nature 520: 186-191, incorporated herein by reference), which improves the cloning and manipulation of the Cas9 components in vitro. The technology encompasses embodiments comprising use of these compact genes fused to a nuclease, e.g., Fok1.

In some embodiments, different Cas9 proteins (e.g., Cas9 proteins from various species and modified versions (e.g., nuclease-deficient versions) thereof) may be advantageous to use in the various provided methods in order to capitalize on various characteristics of the different Cas9 proteins (e.g., for different PAM sequence preferences; for no PAM sequence requirement; for increased or decreased binding activity; for an increased or decreased level of cellular toxicity; for increase or decrease efficiency of in vitro RNP formation; for increase or decrease ability for introduction into cells (e.g., living cells, e.g., living primary cells), etc.). Cas9 proteins from various species may require different PAM sequences in the target DNA. Thus, for a particular Cas9 protein of choice, the PAM sequence requirement may be different than the 5′-XGG-3′ sequence described above. In some embodiments, the protein is an xCas protein having an expanded PAM compatibility (e.g., a Cas9 variant that recognizes a broad range of PAM sequences including NG, GAA and GAT), e.g., as described in Hu et al. (2018) “Evolved Cas9 variants with broad PAM compatibility and high DNA specificity” Nature 556: 57-63, incorporated herein by reference in its entirety.

In some embodiments, the technology comprises use of other Cas9-like RNA-guided nucleases (e.g., Cpf1 and modified versions thereof) and DNA-binding domains thereof. For example, in some embodiments, use of other RNA-guide nucleases (e.g., Cpf1 and modified versions thereof) provides advantages—e.g., in some embodiments the characteristics of the different nucleases are appropriate for methods as described herein (e.g., other RNA-guided nucleases have preferences for different PAM sequence preferences; other RNA-guided nucleases operate using single crRNAs other than cr/tracrRNA complexes; other RNA-guided nucleases operate with shorter guide RNAs, etc.) In some embodiments, the technology comprises use of a Cpf1 enzyme, e.g., as described in U.S. Pat. No. 9,790,490, which is incorporated herein by reference in its entirety.

Many Cas9 orthologs from a wide variety of species have been identified herein and the proteins share only a few identical amino acids. All identified Cas9 orthologs have the same domain architecture with a central HNH endonuclease domain and a split RuvC/RNaseH domain. Cas9 proteins share 4 key motifs with a conserved architecture. Motifs 1, 2, and 4 are RuvC like motifs while motif 3 is an HNH-motif. In some embodiments, a suitable polypeptide (e.g., a Cas9) comprises an amino acid sequence having 4 motifs, each of motifs 1-4 having at least about 75%, at least about 80%, at least about 85%, at least about 90%, at least about 95%, at least about 99% or 100% amino acid sequence identity to the motifs 1-4 of a known Cas9 and/or Csn1 amino acid sequence.

A number of bacteria express Cas9 protein variants. The Cas9 from Streptococcus pyogenes is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12; 2013, incorporated herein by reference), and a large number of Cas9 proteins are listed in supplementary FIG. 1 and supplementary table 1 thereof, which are incorporated by reference herein. Additional Cas9 proteins are described in Esvelt et al., Nat Methods. 2013 November; 10(11):1116-21 and Fonfara et al., “Phylogeny of Cas9 determines functional exchangeability of dual-RNA and Cas9 among orthologous type II CRISPR-Cas systems.” Nucleic Acids Res. 42: 2577-90 (2014), each of which is incorporated herein by reference.

Cas9, and thus dCas9-Fok1, molecules of a variety of species find use in the technology described herein. While the S. pyogenes and S. thermophilus Cas9 molecules are widely used, Cas9 molecules of, derived from, or based on the Cas9 proteins of other species listed herein find use in embodiments of the technology. Accordingly, the technology provides for the replacement of S. pyogenes and S. thermophilus Cas9 and dCas9-Fok1 molecules with Cas9 and dCas9-Fok1 molecules produced by and/or derived from other species, e.g:

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

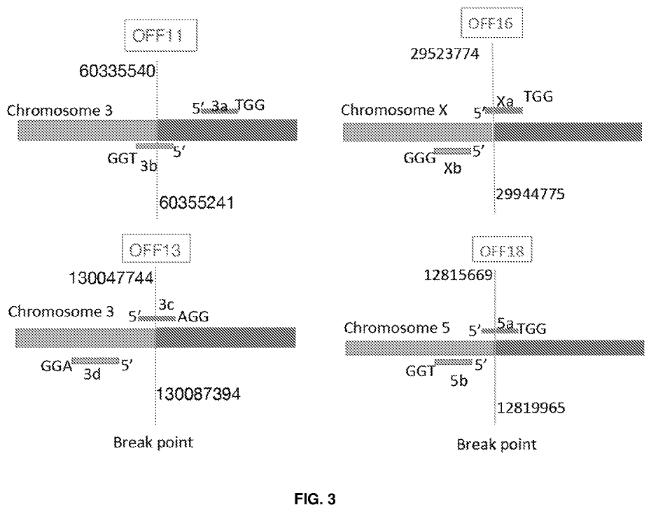

See also U.S. Pat. App. Pub. No. 20170051312 at FIGS. 3, 4, 5, incorporated herein by reference.

In some embodiments, the technology described herein encompasses the use of a dCas9-Fok1 fusion protein derived from any Cas9 protein (e.g., as listed above) and their corresponding guide RNAs or other guide RNAs that are compatible. The Cas9 from the Streptococcus thermophilus LMD-9 CRISPR1 system has been shown to function in human cells (see, e.g., Cong et al. (2013) Science 339: 819, incorporated herein by reference). Additionally, Jinek showed in vitro that Cas9 orthologs from S. thermophilus and L. innocua, can be guided by a dual S. pyogenes gRNA to cleave target plasmid DNA.

In some embodiments, the present technology comprises a polypeptide that is, that comprises, that is similar to, or that comprises a portion that is or is similar to, the Cas9 protein from S. pyogenes, either as encoded in bacteria or codon-optimized for expression in mammalian cells. For example, in some embodiments, the Cas9 used herein is at least approximately 50% identical to the sequence of S. pyogenes Cas9, e.g., at least 50% identical to the following sequence (SEQ ID NO: 2).

```
Met Asp Lys Lys Tyr Ser Ile Gly Leu Asp Ile Gly Thr Asn Ser Val
1 5 10 15
Gly Trp Ala Val Ile Thr Asp Glu Tyr Lys Val Pro Ser Lys Lys Phe
                        20 25 30
Lys Val Leu Gly Asn Thr Asp Arg His Ser Ile Lys Lys Asn Leu Ile
                35 40 45
Gly Ala Leu Leu Phe Asp Ser Gly Glu Thr Ala Glu Ala Thr Arg Leu
        50 55 60
Lys Arg Thr Ala Arg Arg Arg Tyr Thr Arg Arg Lys Asn Arg Ile Cys
65 70 75 80
Tyr Leu Gln Glu Ile Phe Ser Asn Glu Met Ala Lys Val Asp Asp Ser
                                85 90 95
Phe Phe His Arg Leu Glu Glu Ser Phe Leu Val Glu Glu Asp Lys Lys
                        100 105 110
His Glu Arg His Pro Ile Phe Gly Asn Ile Val Asp Glu Val Ala Tyr
                115 120 125
His Glu Lys Tyr Pro Thr Ile Tyr His Leu Arg Lys Lys Leu Val Asp
        130 135 140
Ser Thr Asp Lys Ala Asp Leu Arg Leu Ile Tyr Leu Ala Leu Ala His
145 150 155 160
Met Ile Lys Phe Arg Gly His Phe Leu Ile Glu Gly Asp Leu Asn Pro
                              165 170 175
Asp Asn Ser Asp Val Asp Lys Leu Phe Ile Gln Leu Val Gln Thr Tyr
                      180 185 190
Asn Gln Leu Phe Glu Glu Asn Pro Ile Asn Ala Ser Gly Val Asp Ala
                195 200 205
Lys Ala Ile Leu Ser Ala Arg Leu Ser Lys Ser Arg Arg Leu Glu Asn
      210 215 220
Leu Ile Ala Gln Leu Pro Gly Glu Lys Lys Asn Gly Leu Phe Gly Asn
225 230 235 240
Leu Ile Ala Leu Ser Leu Gly Leu Thr Pro Asn Phe Lys Ser Asn Phe
                                245 250 255
Asp Leu Ala Glu Asp Ala Lys Leu Gln Leu Ser Lys Asp Thr Tyr Asp
                        260 265 270
Asp Asp Leu Asp Asn Leu Leu Ala Gln Ile Gly Asp Gln Tyr Ala Asp
                275 280 285
Leu Phe Leu Ala Ala Lys Asn Leu Ser Asp Ala Ile Leu Leu Ser Asp
        290 295 300
Ile Leu Arg Val Asn Thr Glu Ile Thr Lys Ala Pro Leu Ser Ala Ser
305 310 315 320
Met Ile Lys Arg Tyr Asp Glu His His Gln Asp Leu Thr Leu Leu Lys
                                325 330 335
Ala Leu Val Arg Gln Gln Leu Pro Glu Lys Tyr Lys Glu Ile Phe Phe
                        340 345 350
Asp Gln Ser Lys Asn Gly Tyr Ala Gly Tyr Ile Asp Gly Gly Ala Ser
                355 360 365
Gln Glu Glu Phe Tyr Lys Phe Ile Lys Pro Ile Leu Glu Lys Met Asp
        370 375 380
Gly Thr Glu Glu Leu Leu Val Lys Leu Asn Arg Glu Asp Leu Leu Arg
385 390 395 400
Lys Gln Arg Thr Phe Asp Asn Gly Ser Ile Pro His Gln Ile His Leu
                                405 410 415
Gly Glu Leu His Ala Ile Leu Arg Arg Gln Glu Asp Phe Tyr Pro Phe
                        420 425 430
Leu Lys Asp Asn Arg Glu Lys Ile Glu Lys Ile Leu Thr Phe Arg Ile
                435 440 445
Pro Tyr Tyr Val Gly Pro Leu Ala Arg Gly Asn Ser Arg Phe Ala Trp
        450 455 460
Met Thr Arg Lys Ser Glu Glu Thr Ile Thr Pro Trp Asn Phe Glu Glu
465 470 475 480
Val Val Asp Lys Gly Ala Ser Ala Gln Ser Phe Ile Glu Arg Met Thr
                                485 490 495
Asn Phe Asp Lys Asn Leu Pro Asn Glu Lys Val Leu Pro Lys His Ser
                        500 505 510
Leu Leu Tyr Glu Tyr Phe Thr Val Tyr Asn Glu Leu Thr Lys Val Lys
                515 520 525
Tyr Val Thr Glu Gly Met Arg Lys Pro Ala Phe Leu Ser Gly Glu Gln
        530 535 540
Lys Lys Ala Ile Val Asp Leu Leu Phe Lys Thr Asn Arg Lys Val Thr
545 550 555 560
Val Lys Gln Leu Lys Glu Asp Tyr Phe Lys Lys Ile Glu Cys Phe Asp
                                565 570 575
Ser Val Glu Ile Ser Gly Val Glu Asp Arg Phe Asn Ala Ser Leu Gly
                        580 585 590
Thr Tyr His Asp Leu Leu Lys Ile Ile Lys Asp Lys Asp Phe Leu Asp
                595 600 605
Asn Glu Glu Asn Glu Asp Ile Leu Glu Asp Ile Val Leu Thr Leu Thr
        610 615 620
Leu Phe Glu Asp Arg Glu Met Ile Glu Glu Arg Leu Lys Thr Tyr Ala
625 630 635 640
His Leu Phe Asp Asp Lys Val Met Lys Gln Leu Lys Arg Arg Arg Tyr
                                645 650 655
Thr Gly Trp Gly Arg Leu Ser Arg Lys Leu Ile Asn Gly Ile Arg Asp
                        660 665 670
Lys Gln Ser Gly Lys Thr Ile Leu Asp Phe Leu Lys Ser Asp Gly Phe
                675 680 685
Ala Asn Arg Asn Phe Met Gln Leu Ile His Asp Asp Ser Leu Thr Phe
        690 695 700
Lys Glu Asp Ile Gln Lys Ala Gln Val Ser Gly Gln Gly Asp Ser Leu
705 710 715 720
His Glu His Ile Ala Asn Leu Ala Gly Ser Pro Ala Ile Lys Lys Gly
                                725 730 735
Ile Leu Gln Thr Val Lys Val Val Asp Glu Leu Val Lys Val Met Gly
                        740 745 750
Arg His Lys Pro Glu Asn Ile Val Ile Glu Met Ala Arg Glu Asn Gln
                755 760 765
Thr Thr Gln Lys Gly Gln Lys Asn Ser Arg Glu Arg Met Lys Arg Ile
        770 775 780
Glu Glu Gly Ile Lys Glu Leu Gly Ser Gln Ile Leu Lys Glu His Pro
785 790 795 800
Val Glu Asn Thr Gln Leu Gln Asn Glu Lys Leu Tyr Leu Tyr Tyr Leu
                                805 810 815
Gln Asn Gly Arg Asp Met Tyr Val Asp Gln Glu Leu Asp Ile Asn Arg
                        820 825 830
Leu Ser Asp Tyr Asp Val Asp His Ile Val Pro Gln Ser Phe Leu Lys
                835 840 845
Asp Asp Ser Ile Asp Asn Lys Val Leu Thr Arg Ser Asp Lys Asn Arg
        850 855 860
Gly Lys Ser Asp Asn Val Pro Ser Glu Glu Val Val Lys Lys Met Lys
865 870 875 880
Asn Tyr Trp Arg Gln Leu Leu Asn Ala Lys Leu Ile Thr Gln Arg Lys
                                885 890 895
Phe Asp Asn Leu Thr Lys Ala Glu Arg Gly Gly Leu Ser Glu Leu Asp
                        900 905 910
Lys Ala Gly Phe Ile Lys Arg Gln Leu Val Glu Thr Arg Gln Ile Thr
                915 920 925
Lys His Val Ala Gln Ile Leu Asp Ser Arg Met Asn Thr Lys Tyr Asp
        930 935 940
Glu Asn Asp Lys Leu Ile Arg Glu Val Lys Val Ile Thr Leu Lys Ser
945 950 955 960
Lys Leu Val Ser Asp Phe Arg Lys Asp Phe Gln Phe Tyr Lys Val Arg
                                965 970 975
Glu Ile Asn Asn Tyr His His Ala His Asp Ala Tyr Leu Asn Ala Val
                        980 985 990
Val Gly Thr Ala Leu Ile Lys Lys Tyr Pro Lys Leu Glu Ser Glu Phe
                995 1000 1005
Val Tyr Gly Asp Tyr Lys Val Tyr Asp Val Arg Lys Met Ile Ala
      1010 1015 1020
Lys Ser Glu Gln Glu Ile Gly Lys Ala Thr Ala Lys Tyr Phe Phe
        1025 1030 1035
Tyr Ser Asn Ile Met Asn Phe Phe Lys Thr Glu Ile Thr Leu Ala
      1040 1045 1050
Asn Gly Glu Ile Arg Lys Arg Pro Leu Ile Glu Thr Asn Gly Glu
      1055 1060 1065
Thr Gly Glu Ile Val Trp Asp Lys Gly Arg Asp Phe Ala Thr Val
      1070 1075 1080
Arg Lys Val Leu Ser Met Pro Gln Val Asn Ile Val Lys Lys Thr
      1085 1090 1095
Glu Val Gln Thr Gly Gly Phe Ser Lys Glu Ser Ile Leu Pro Lys
      1100 1105 1110
Arg Asn Ser Asp Lys Leu Ile Ala Arg Lys Lys Asp Trp Asp Pro
      1115 1120 1125
Lys Lys Tyr Gly Gly Phe Asp Ser Pro Thr Val Ala Tyr Ser Val
      1130 1135 1140
Leu Val Val Ala Lys Val Glu Lys Gly Lys Ser Lys Lys Leu Lys
      1145 1150 1155
Ser Val Lys Glu Leu Leu Gly Ile Thr Ile Met Glu Arg Ser Ser
      1160 1165 1170
Phe Glu Lys Asn Pro Ile Asp Phe Leu Glu Ala Lys Gly Tyr Lys
      1175 1180 1185
Glu Val Lys Lys Asp Leu Ile Ile Lys Leu Pro Lys Tyr Ser Leu
      1190 1195 1200
Phe Glu Leu Glu Asn Gly Arg Lys Arg Met Leu Ala Ser Ala Gly
      1205 1210 1215
Glu Leu Gln Lys Gly Asn Glu Leu Ala Leu Pro Ser Lys Tyr Val
      1220 1225 1230
Asn Phe Leu Tyr Leu Ala Ser His Tyr Glu Lys Leu Lys Gly Ser
      1235 1240 1245
Pro Glu Asp Asn Glu Gln Lys Gln Leu Phe Val Glu Gln His Lys
      1250 1255 1260
His Tyr Leu Asp Glu Ile Ile Glu Gln Ile Ser Glu Phe Ser Lys
      1265 1270 1275
Arg Val Ile Leu Ala Asp Ala Asn Leu Asp Lys Val Leu Ser Ala
      1280 1285 1290
Tyr Asn Lys His Arg Asp Lys Pro Ile Arg Glu Gln Ala Glu Asn
      1295 1300 1305
Ile Ile His Leu Phe Thr Leu Thr Asn Leu Gly Ala Pro Ala Ala
      1310 1315 1320
Phe Lys Tyr Phe Asp Thr Thr Ile Asp Arg Lys Arg Tyr Thr Ser
      1325 1330 1335
Thr Lys Glu Val Leu Asp Ala Thr Leu Ile His Gln Ser Ile Thr
      1340 1345 1350
Gly Leu Tyr Glu Thr Arg Ile Asp Leu Ser Gln Leu Gly Gly Asp
      1355 1360 1365
```

In some embodiments, the technology comprises use of a nucleotide sequence that is approximately 50%, 55%, 60%, 65%, 70%, 75%, 80%, 85%, 90%, 95%, 99% or 100% identical to a nucleotide sequence that encodes a protein described by SEQ ID NO: 2.

In some embodiments, the Cas9 portion of the dCas9-Fok1 fusion protein used herein is at least about 50% identical to the sequence of the S. pyogenes Cas9, e.g., at least 50%, 55%, 60%, 65%, 70%, 75%, 80%, 85%, 90%, 95%, 99% or 100% identical to SEQ ID NO: 2.

In some embodiments, the present technology comprises use of a catalytically inactive form of a Cas9 or Cas9-like protein (“dead Cas9” or “dCas9”). In some embodiments, the dCas9 or dCas9-like protein comprises point mutations (e.g., introduced by genetic engineering, molecular biology, and/or other recombinant nucleic acid technologies) that disable the nuclease activity. In some embodiments, the dCas9 protein is from S. pyogenes. In some embodiments, the dCas9 protein comprises mutations at, e.g., D10, E762, H983, and/or D986; and at H840 and/or N863 (e.g., at D10 and H840 (e.g., comprising D10A or D10N and H840A or H840N or H840Y)). In some embodiments, the present technology comprises the Cas9 protein from S. pyogenes, either as encoded in bacteria or codon-optimized for expression in mammalian cells, containing mutations at D10, E762, H983, or D986 and H840 or N863, e.g., D10A/D10N and H840A/H840N/H840Y, to render the nuclease portion of the protein catalytically inactive; substitutions at these positions are, in some embodiments, alanine (Nishimasu (2014) Cell 156: 935-949) or, in some embodiments, other residues, e.g., glutamine, asparagine, tyrosine, serine, or aspartate, e.g., E762Q, H983N, H983Y, D986N, N863D, N863S, or N863H. The sequence of one S. pyogenes dCas9 protein that finds use in embodiments of the technology provided herein is described in US20160010076, which is incorporated herein by reference in its entirety.

In some embodiments, the present technology comprises a polypeptide that is, that comprises, that is similar to, or that comprises a portion that is or is similar to, the sequence of S. pyogenes Cas9, e.g., at least 50% identical to the following sequence of dCas9 comprising the D10A and H840A substitutions (SEQ ID NO: 3). For example, in some embodiments, the dCas9 used herein is at least about 50% identical to the sequence of S. pyogenes Cas9, e.g., at least 50% identical to the following sequence of dCas9 comprising the D10A and H840A substitutions (SEQ ID NO: 3).

```
Met Asp Lys Lys Tyr Ser Ile Gly Leu Ala Ile Gly Thr Asn Ser Val
1 5 10 15
Gly Trp Ala Val Ile Thr Asp Glu Tyr Lys Val Pro Ser Lys Lys Phe
                        20 25 30
Lys Val Leu Gly Asn Thr Asp Arg His Ser Ile Lys Lys Asn Leu Ile
                35 40 45
Gly Ala Leu Leu Phe Asp Ser Gly Glu Thr Ala Glu Ala Thr Arg Leu
        50 55 60
Lys Arg Thr Ala Arg Arg Arg Tyr Thr Arg Arg Lys Asn Arg Ile Cys
65 70 75 80
Tyr Leu Gln Glu Ile Phe Ser Asn Glu Met Ala Lys Val Asp Asp Ser
                                85 90 95
Phe Phe His Arg Leu Glu Glu Ser Phe Leu Val Glu Glu Asp Lys Lys
                      100 105 110
His Glu Arg His Pro Ile Phe Gly Asn Ile Val Asp Glu Val Ala Tyr
                115 120 125
His Glu Lys Tyr Pro Thr Ile Tyr His Leu Arg Lys Lys Leu Val Asp
        130 135 140
Ser Thr Asp Lys Ala Asp Leu Arg Leu Ile Tyr Leu Ala Leu Ala His
145 150 155 160
Met Ile Lys Phe Arg Gly His Phe Leu Ile Glu Gly Asp Leu Asn Pro
                                165 170 175
Asp Asn Ser Asp Val Asp Lys Leu Phe Ile Gln Leu Val Gln Thr Tyr
                        180 185 190
Asn Gln Leu Phe Glu Glu Asn Pro Ile Asn Ala Ser Gly Val Asp Ala
                195 200 205
Lys Ala Ile Leu Ser Ala Arg Leu Ser Lys Ser Arg Arg Leu Glu Asn
        210 215 220
Leu Ile Ala Gln Leu Pro Gly Glu Lys Lys Asn Gly Leu Phe Gly Asn
225 230 235 240
Leu Ile Ala Leu Ser Leu Gly Leu Thr Pro Asn Phe Lys Ser Asn Phe
                                245 250 255
Asp Leu Ala Glu Asp Ala Lys Leu Gln Leu Ser Lys Asp Thr Tyr Asp
                        260 265 270
Asp Asp Leu Asp Asn Leu Leu Ala Gln Ile Gly Asp Gln Tyr Ala Asp
                275 280 285
Leu Phe Leu Ala Ala Lys Asn Leu Ser Asp Ala Ile Leu Leu Ser Asp
        290 295 300
Ile Leu Arg Val Asn Thr Glu Ile Thr Lys Ala Pro Leu Ser Ala Ser
305 310 315 320
Met Ile Lys Arg Tyr Asp Glu His His Gln Asp Leu Thr Leu Leu Lys
                                325 330 335
Ala Leu Val Arg Gln Gln Leu Pro Glu Lys Tyr Lys Glu Ile Phe Phe
                        340 345 350
Asp Gln Ser Lys Asn Gly Tyr Ala Gly Tyr Ile Asp Gly Gly Ala Ser
                355 360 365
Gln Glu Glu Phe Tyr Lys Phe Ile Lys Pro Ile Leu Glu Lys Met Asp
        370 375 380
Gly Thr Glu Glu Leu Leu Val Lys Leu Asn Arg Glu Asp Leu Leu Arg
385 390 395 400
Lys Gln Arg Thr Phe Asp Asn Gly Ser Ile Pro His Gln Ile His Leu
                                405 410 415
Gly Glu Leu His Ala Ile Leu Arg Arg Gln Glu Asp Phe Tyr Pro Phe
                        420 425 430
Leu Lys Asp Asn Arg Glu Lys Ile Glu Lys Ile Leu Thr Phe Arg Ile
              435 440 445
Pro Tyr Tyr Val Gly Pro Leu Ala Arg Gly Asn Ser Arg Phe Ala Trp
      450 455 460
Met Thr Arg Lys Ser Glu Glu Thr Ile Thr Pro Trp Asn Phe Glu Glu
465 470 475 480
Val Val Asp Lys Gly Ala Ser Ala Gln Ser Phe Ile Glu Arg Met Thr
                                485 490 495
Asn Phe Asp Lys Asn Leu Pro Asn Glu Lys Val Leu Pro Lys His Ser
                        500 505 510
Leu Leu Tyr Glu Tyr Phe Thr Val Tyr Asn Glu Leu Thr Lys Val Lys
                515 520 525
Tyr Val Thr Glu Gly Met Arg Lys Pro Ala Phe Leu Ser Gly Glu Gln
        530 535 540
Lys Lys Ala Ile Val Asp Leu Leu Phe Lys Thr Asn Arg Lys Val Thr
545 550 555 560
Val Lys Gln Leu Lys Glu Asp Tyr Phe Lys Lys Ile Glu Cys Phe Asp
                                565 570 575
Ser Val Glu Ile Ser Gly Val Glu Asp Arg Phe Asn Ala Ser Leu Gly
                        580 585 590
Thr Tyr His Asp Leu Leu Lys Ile Ile Lys Asp Lys Asp Phe Leu Asp
                595 600 605
Asn Glu Glu Asn Glu Asp Ile Leu Glu Asp Ile Val Leu Thr Leu Thr
        610 615 620
Leu Phe Glu Asp Arg Glu Met Ile Glu Glu Arg Leu Lys Thr Tyr Ala
625 630 635 640
His Leu Phe Asp Asp Lys Val Met Lys Gln Leu Lys Arg Arg Arg Tyr
                              645 650 655
Thr Gly Trp Gly Arg Leu Ser Arg Lys Leu Ile Asn Gly Ile Arg Asp
                        660 665 670
Lys Gln Ser Gly Lys Thr Ile Leu Asp Phe Leu Lys Ser Asp Gly Phe
                675 680 685
Ala Asn Arg Asn Phe Met Gln Leu Ile His Asp Asp Ser Leu Thr Phe
        690 695 700
Lys Glu Asp Ile Gln Lys Ala Gln Val Ser Gly Gln Gly Asp Ser Leu
705 710 715 720
His Glu His Ile Ala Asn Leu Ala Gly Ser Pro Ala Ile Lys Lys Gly
                                725 730 735
Ile Leu Gln Thr Val Lys Val Val Asp Glu Leu Val Lys Val Met Gly
                        740 745 750
Arg His Lys Pro Glu Asn Ile Val Ile Glu Met Ala Arg Glu Asn Gln
                755 760 765
Thr Thr Gln Lys Gly Gln Lys Asn Ser Arg Glu Arg Met Lys Arg Ile
        770 775 780
Glu Glu Gly Ile Lys Glu Leu Gly Ser Gln Ile Leu Lys Glu His Pro
785 790 795 800
Val Glu Asn Thr Gln Leu Gln Asn Glu Lys Leu Tyr Leu Tyr Tyr Leu
                                805 810 815
Gln Asn Gly Arg Asp Met Tyr Val Asp Gln Glu Leu Asp Ile Asn Arg
                        820 825 830
Leu Ser Asp Tyr Asp Val Asp Ala Ile Val Pro Gln Ser Phe Leu Lys
                835 840 845
Asp Asp Ser Ile Asp Asn Lys Val Leu Thr Arg Ser Asp Lys Asn Arg
      850 855 860
Gly Lys Ser Asp Asn Val Pro Ser Glu Glu Val Val Lys Lys Met Lys
865 870 875 880
Asn Tyr Trp Arg Gln Leu Leu Asn Ala Lys Leu Ile Thr Gln Arg Lys
                                885 890 895
Phe Asp Asn Leu Thr Lys Ala Glu Arg Gly Gly Leu Ser Glu Leu Asp
                        900 905 910
Lys Ala Gly Phe Ile Lys Arg Gln Leu Val Glu Thr Arg Gln Ile Thr
                915 920 925
Lys His Val Ala Gln Ile Leu Asp Ser Arg Met Asn Thr Lys Tyr Asp
        930 935 940
Glu Asn Asp Lys Leu Ile Arg Glu Val Lys Val Ile Thr Leu Lys Ser
945 950 955 960
Lys Leu Val Ser Asp Phe Arg Lys Asp Phe Gln Phe Tyr Lys Val Arg
                                965 970 975
Glu Ile Asn Asn Tyr His His Ala His Asp Ala Tyr Leu Asn Ala Val
                        980 985 990
Val Gly Thr Ala Leu Ile Lys Lys Tyr Pro Lys Leu Glu Ser Glu Phe
                995 1000 1005
Val Tyr Gly Asp Tyr Lys Val Tyr Asp Val Arg Lys Met Ile Ala
      1010 1015 1020
Lys Ser Glu Gln Glu Ile Gly Lys Ala Thr Ala Lys Tyr Phe Phe
      1025 1030 1035
Tyr Ser Asn Ile Met Asn Phe Phe Lys Thr Glu Ile Thr Leu Ala
      1040 1045 1050
Asn Gly Glu Ile Arg Lys Arg Pro Leu Ile Glu Thr Asn Gly Glu
      1055 1060 1065
Thr Gly Glu Ile Val Trp Asp Lys Gly Arg Asp Phe Ala Thr Val
      1070 1075 1080
Arg Lys Val Leu Ser Met Pro Gln Val Asn Ile Val Lys Lys Thr
      1085 1090 1095
Glu Val Gln Thr Gly Gly Phe Ser Lys Glu Ser Ile Leu Pro Lys
      1100 1105 1110
Arg Asn Ser Asp Lys Leu Ile Ala Arg Lys Lys Asp Trp Asp Pro
      1115 1120 1125
Lys Lys Tyr Gly Gly Phe Asp Ser Pro Thr Val Ala Tyr Ser Val
      1130 1135 1140
Leu Val Val Ala Lys Val Glu Lys Gly Lys Ser Lys Lys Leu Lys
      1145 1150 1155
Ser Val Lys Glu Leu Leu Gly Ile Thr Ile Met Glu Arg Ser Ser
      1160 1165 1170
Phe Glu Lys Asn Pro Ile Asp Phe Leu Glu Ala Lys Gly Tyr Lys
      1175 1180 1185
Glu Val Lys Lys Asp Leu Ile Ile Lys Leu Pro Lys Tyr Ser Leu
      1190 1195 1200
Phe Glu Leu Glu Asn Gly Arg Lys Arg Met Leu Ala Ser Ala Gly
      1205 1210 1215
Glu Leu Gln Lys Gly Asn Glu Leu Ala Leu Pro Ser Lys Tyr Val
      1220 1225 1230
Asn Phe Leu Tyr Leu Ala Ser His Tyr Glu Lys Leu Lys Gly Ser
      1235 1240 1245
Pro Glu Asp Asn Glu Gln Lys Gln Leu Phe Val Glu Gln His Lys
      1250 1255 1260
His Tyr Leu Asp Glu Ile Ile Glu Gln Ile Ser Glu Phe Ser Lys
      1265 1270 1275
Arg Val Ile Leu Ala Asp Ala Asn Leu Asp Lys Val Leu Ser Ala
      1280 1285 1290
Tyr Asn Lys His Arg Asp Lys Pro Ile Arg Glu Gln Ala Glu Asn
      1295 1300 1305
Ile Ile His Leu Phe Thr Leu Thr Asn Leu Gly Ala Pro Ala Ala
      1310 1315 1320
Phe Lys Tyr Phe Asp Thr Thr Ile Asp Arg Lys Arg Tyr Thr Ser
      1325 1330 1335
Thr Lys Glu Val Leu Asp Ala Thr Leu Ile His Gln Ser Ile Thr
      1340 1345 1350
Gly Leu Tyr Glu Thr Arg Ile Asp Leu Ser Gln Leu Gly Gly Asp
      1355 1360 1365
```

In some embodiments, the technology comprises use of a nucleotide sequence that is approximately 50%, 55%, 60%, 65%, 70%, 75%, 80%, 85%, 90%, 95%, 99% or 100% identical to a nucleotide sequence that encodes a protein described by SEQ ID NO: 3.

In some embodiments, the dCas9 used herein is at least about 50% identical to the sequence of the catalytically inactive S. pyogenes Cas9, i.e., at least 50%, 55%, 60%, 65%, 70%, 75%, 80%, 85%, 90%, 95%, 99% or 100% identical to SEQ ID NO:1, wherein the mutations at D10 and H840, e.g., D10A/D10N and H840A/H840N/H840Y are maintained.

In some embodiments, any differences from SEQ ID NO:1 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11)1116-21 and Fonfara et al., Nucl. Acids Res. (2014) 42 (4): 2577-2590, and wherein the mutations at D10 and H840, e.g., D10A/D10N and H840A/H840N/H840Y are maintained.

In exemplary embodiments, the technology provides a gRNA-guided nuclease (e.g., a fusion protein) comprising a nucleic acid-binding component (e.g., a dCas9 or portion thereof) and a nuclease (e.g., a Fok1 or portion thereof). In some embodiments, the gRNA-guided nuclease binds a guide RNA (gRNA).

In some embodiments, the technology provides a polypeptide (e.g., a dCas9-Fok1) comprising a Cas protein, CRISPR enzyme, Cas-like protein, or domain thereof (e.g., a dead Cas protein, CRISPR enzyme, Cas-like protein, or domain thereof). “Cas protein” and “CRISPR enzyme” and “Cas-like protein”, as used herein, includes polypeptides, enzymatic activities, and polypeptides having activities similar to proteins known in the art as, or encoded by genes known in the art as, e.g., Cas1, Cas1B, Cast, Cas3, Cas4, Cas5, Cas6, Cas7, Cas8, Cas9 (also known as Csn1 and Csx12), Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, homologs thereof, or modified versions thereof, e.g., including fusions of a nuclease (e.g., Fok1) with any of these Cas proteins, CRISPR enzymes, and/or Cas-like proteins known in the art.

In some embodiments, the technology comprises use of a polypeptide (e.g., a Type V/Type VI protein) such as Cpf1 or C2c1 or C2c2 and homologs and orthologs of a Type V/Type VI protein such as Cpf1 or C2c1 or C2c2 to provide a fusion with a nuclease (e.g., Fok1). Embodiments encompass Cpf1, modified Cpf1 (e.g., Cpf1-Fok1 fusion), and Cpf1, and CRISPR systems related to Cpf1, modified Cpf1 (Cpf1-Fok1 fusion), and chimeric Cpf1. In some embodiments, the polypeptide (e.g., a Type V/Type VI protein) such as Cpf1 or C2c1 or C2c2 is from a genus that is, e.g., Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter; Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacteriurn, or Acidaminococcus. In some embodiments, the polypeptide (e.g., a Type V/Type VI protein) such as Cpf1 or C2c1 or C2c2 is from an organism that is, e.g., S. mutans, S. agalactiae, S. equisimills, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, or C. sordellii. See, e.g., U.S. Pat. No. 9,790,490, incorporated herein by reference in its entirety. In some embodiments, a gRNA-targeted nuclease comprises a Cpf1 protein as described in U.S. Pat. App. Pub. No. 20180155716, which is incorporated herein by reference.

In some embodiments, proteins of the technology comprise differences in their amino acid sequence relative to SEQ ID NO: 2 in non-conserved regions, e.g., as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11)1116-21; and/or Fonfara et al., Nucl. Acids Res. (2014) 42 (4): 2577-2590, each of which is incorporated herein by reference.

Thus, in some embodiments, the polypeptide of the Cas9 portion of the RNP is a naturally-occurring polypeptide. In some embodiments, the polypeptide of the Cas9 portion of the RNP is not a naturally-occurring polypeptide (e.g., a chimeric polypeptide, a naturally-occurring polypeptide that is modified, e.g., by one or more amino acid substitutions produced by an engineered nucleic acid comprising one or more nucleotide substitutions, deletions, insertions).

In some embodiments, choosing, designing, synthesizing, and analyzing nucleotide sequences and amino acid sequences (e.g., of the polypeptide and RNA components of an RNP complex as described herein) comprise use of sequence alignment methods to identify similarities and differences in two or more nucleotide sequences or amino acid sequences. To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence). The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch ((1970) J. Mol. Biol. 48:444-453, incorporated herein by reference) algorithm, which has been incorporated into the GAP program in the GCG software package, e.g., using a Blosum 62 scoring matrix with a gap penalty of 12, a gap extend penalty of 4, and a frameshift gap penalty of 5. Other methods are known in the art, e.g., as discussed elsewhere herein.

In some embodiments, the RNP comprises a protein that is a Cas9 or Cas9 derivative, e.g., a Cas9-Fok1 fusion. Thus, in some embodiments, the protein is a Type II Cas9 protein. In some embodiments, the Cas9 has been engineered to modify (e.g., remove, partially inactivate, and/or totally inactivate) the nuclease domain (e.g., to provide a “dead Cas9” (dCas9) or a “Cas9 nickase”; see, e.g., Nature Methods 11: 399-402 (2014), incorporated herein by reference). In some embodiments, the RNP protein is a protein from a CRISPR system other than the S. pyogenes system, e.g., a Type V Cpf1, C2c1, C2c2, C2c3 protein, or derivative thereof.

In some embodiments, the polypeptide of the RNP is a chimeric or fusion polypeptide, e.g., a polypeptide that comprises two or more functional domains (e.g., a gRNA-guided DNA-binding domain (e.g., dCas9) and a nuclease (e.g., Fok1) domain). For example, in some embodiments a chimeric polypeptide interacts with (e.g., binds to) an RNA to form an RNP (described above). The RNA guides the fusion polypeptide to a target sequence within a target DNA (e.g., nucleic acid rearrangement junction (e.g., chromosome rearrangement junction (CRJ), extrachromosomal circle junction)). Thus, in some embodiments a chimeric polypeptide binds target DNA.

A chimeric or fusion polypeptide comprises at least two portions, e.g., an RNA binding portion and an “activity” portion (e.g., a nuclease). A chimeric or fusion polypeptide comprises amino acid sequences that are derived from at least two different polypeptides. A chimeric or fusion polypeptide can comprise modified and/or naturally occurring polypeptide sequences (e.g., a first amino acid sequence from a modified or unmodified Cas9/Csn1 protein; and a second amino acid sequence other than the Cas9/Csn1 protein, e.g., a nuclease (e.g., Fok1) domain).

In some embodiments, the RNA-binding portion of a chimeric polypeptide is a naturally-occurring polypeptide. In some embodiments, the RNA-binding portion of a chimeric polypeptide is not a naturally-occurring molecule (e.g., modified with respect to a naturally-occurring polypeptide by, e.g., substitution, deletion, insertion). In some embodiments, naturally-occurring RNA-binding portions of interest are derived from polypeptides known in the art, e.g., discussed herein (e.g., Cas9 and similar polypeptides).

In some embodiments, the RNA-binding portion of a chimeric polypeptide comprises an amino acid sequence having at least about 75%, at least about 80%, at least about 85%, at least about 90%, at least about 95%, at least about 98%, at least about 99%, or 100% amino acid sequence identity to the RNA-binding portion of a polypeptide described herein.

In some embodiments, the chimeric polypeptide comprises an amino acid sequence having at least about 75%, at least about 80%, at least about 85%, at least about 90%, at least about 95%, at least about 99%, or 100% amino acid sequence identity to a portion of a Cas9 amino acid sequence provided herein.

In addition to the RNA-binding portion, the chimeric polypeptide comprises an “activity portion”, e.g., a nuclease (e.g., Fok1) domain.

A gRNA comprises a first segment (also referred to herein as a “DNA-targeting segment” or a “DNA-targeting sequence”) and a second segment (also referred to herein as a “protein-binding segment” or a “protein-binding sequence”).

The DNA-targeting segment of a gRNA comprises a nucleotide sequence that is complementary to a sequence in a target DNA. In other words, the DNA-targeting segment of a gRNA interacts with a target DNA in a sequence-specific manner via hybridization (e.g., complementary base pairing). As such, the nucleotide sequence of the DNA targeting segment may vary and determines the location within the target DNA that the DNA targeting RNA and the target DNA will interact. The DNA-targeting segment of a gRNA can be modified (e.g., by genetic engineering) to hybridize to any desired sequence within a target DNA.

The DNA-targeting segment (e.g., comprising the DNA-targeting sequence and, in some embodiments, additional nucleic acid) can have a length of from about 8 nucleotides to about 100 nucleotides. For example, the DNA-targeting segment can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the DNA-targeting segment can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

In some embodiments, the nucleotide sequence (the DNA-targeting sequence) of the DNA-targeting segment that is complementary to a nucleotide sequence (target sequence) of the target DNA can have a length at least about 12 nt. For example, the DNA-targeting sequence of the DNA-targeting segment that is complementary to a target sequence of the target DNA can have a length at least about 12 nt, at least about 15 nt, at least about 18 nt, at least about 19 nt, at least about 20 nt, at least about 25 nt, at least about 30 nt, at least about 35 nt, or at least about 40 nt. For example, the DNA-targeting sequence of the DNA-targeting segment that is complementary to a target sequence of the target DNA can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence (the DNA-targeting sequence) of the DNA-targeting segment that is complementary to a nucleotide sequence (target sequence) of the target DNA can have a length at least about 12 nt.

In additional embodiments, the nucleotide sequence (the DNA-targeting sequence) of the DNA-targeting segment that is complementary to a nucleotide sequence (target sequence) of the target DNA can have a length of from about 8 nucleotides to about 30 nucleotides. For example, the DNA-targeting segment can have a length of from about 8 nucleotides (nt) to about 30 nt, from about 8 nt to about 30 nt, from about 8 nt to about 25 nt, from about 8 nt to about 20 nt, from about 8 nt to about 18 nt, from about 8 nt to about 15 nt, or from about 8 nt to about 12 nt, e.g., 8 nt, 9 nt, 10 nt, 11 nt, or 12 nt.

In some embodiments, the DNA-targeting sequence of the DNA-targeting segment that is complementary to a target sequence of the target DNA is 8-20 nucleotides in length. In some embodiments, the DNA-targeting sequence of the DNA-targeting segment that is complementary to a target sequence of the target DNA is 9-12 nucleotides in length.

The percent complementarity between the DNA-targeting sequence of the DNA-targeting segment and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the DNA-targeting segment and the target sequence of the target DNA is 100% over the seven contiguous 5′-most nucleotides of the target sequence of the complementary strand of the target DNA. In some embodiments, the percent complementarity between the DNA-targeting sequence of the DNA-targeting segment and the target sequence of the target DNA is at least 60% over about 20 contiguous nucleotides. In some embodiments, the percent complementarity between the DNA-targeting sequence of the DNA-targeting segment and the target sequence of the target DNA is 100% over the fourteen contiguous 5′-most nucleotides of the target sequence of the complementary strand of the target DNA and as low as 0% over the remainder. In such a case, the DNA-targeting sequence can be considered to be 14 nucleotides in length. In some embodiments, the percent complementarity between the DNA targeting sequence of the DNA-targeting segment and the target sequence of the target DNA is 100% over the seven contiguous 5′-most nucleotides of the target sequence of the complementary strand of the target DNA and as low as 0% over the remainder. In such a case, the DNA-targeting sequence can be considered to be 7 nucleotides in length.

The protein-binding segment of a gRNA interacts with a polypeptide, e.g., a dCas9, dCas9-Fok1, or dCas9-like polypeptide. The gRNA guides the bound polypeptide to a specific nucleotide sequence within target DNA via the above-mentioned DNA-targeting segment. The protein-binding segment of a gRNA comprises two segments comprising nucleotide sequences that are complementary to one another. The complementary nucleotides of the protein-binding segment hybridize to form a double stranded RNA duplex.

A dgRNA comprises two separate RNA molecules. Each of the two RNA molecules of a dgRNA comprises a segment is complementary to one another such that the complementary nucleotides of the two RNA molecules hybridize to form the double stranded RNA duplex of the protein-binding segment.

In some embodiments, the duplex-forming segment of the activator-RNA is at least about 60% identical to one of the activator-RNA (tracrRNA) molecules set forth in U.S. Pat. App. Pub. No. 20170051312, incorporated herein by reference, as SEQ ID NOs: 431-562, or a complement thereof, over a segment of at least 8 contiguous nucleotides. For example, the duplex-forming segment of the activator-RNA (or the DNA encoding the duplex-forming segment of the activator-RNA) is at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to one of the tracrRNA sequences set forth in U.S. Pat. App. Pub. No. 20170051312, incorporated herein by reference, as SEQ ID NOs: 431-562, or a complement thereof, over a segment of at least 8 contiguous nucleotides.

In some embodiments, the duplex-forming segment of the targeter-RNA is at least about 60% identical to one of the targeter-RNA (crRNA) sequences set forth in U.S. Pat. App. Pub. No. 20170051312, incorporated herein by reference, as SEQ ID NOs: 563-679, or a complement thereof, over a segment of at least 8 contiguous nucleotides. For example, the duplex-forming segment of the targeter-RNA (or the DNA encoding the duplex-forming segment of the targeter-RNA) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the crRNA sequences set forth in U.S. Pat. App. Pub. No. 20170051312, incorporated herein by reference, as SEQ ID NOs: 563-679, or a complement thereof, over a segment of at least 8 contiguous nucleotides.

Non-limiting examples of nucleotide sequences that can be included in a two-molecule DNA targeting RNA (dgRNA) include either of the sequences set forth in U.S. Pat. App. Pub. No. 20170051312, incorporated herein by reference, as SEQ ID NOs: 431-562, or complements thereof pairing with any sequences set forth in U.S. Pat. App. Pub. No. 20170051312, incorporated herein by reference, as SEQ ID NOs: 563-679, or complements thereof that can hybridize to form a protein binding segment.

A single-molecule DNA-targeting RNA (sgRNA) comprises two segments of nucleotides (a targeter-RNA and an activator-RNA) that are complementary to one another, are covalently linked by intervening nucleotides (“linkers” or “linker nucleotides”), and hybridize to form the double-stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The linker of a single-molecule DNA-targeting RNA can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single molecule DNA-targeting RNA is 4 nt.

An exemplary single-molecule DNA-targeting RNA comprises two complementary segments of nucleotides that hybridize to form a dsRNA duplex. In some embodiments, one of the two complementary segments of nucleotides of the single-molecule DNA-targeting RNA (or the DNA encoding the segment) is at least about 60% identical to one of the activator-RNA (tracrRNA) molecules set forth in U.S. Pat. App. Pub. No. 20170051312, incorporated herein by reference, as SEQ ID NOs: 431-562, or a complement thereof, over a segment of at least 8 contiguous nucleotides. For example, one of the two complementary segments of nucleotides of the single-molecule DNA-targeting RNA (or the DNA encoding the segment) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the tracrRNA sequences set forth in U.S. Pat. App. Pub. No. 20170051312, incorporated herein by reference, as SEQ ID NOs: 431-562, or a complement thereof, over a segment of at least 8 contiguous nucleotides.

In some embodiments, one of the two complementary segments of nucleotides of the single molecule DNA-targeting RNA (or the DNA encoding the segment) is at least about 60% identical to one of the targeter-RNA (crRNA) sequences set forth in U.S. Pat. App. Pub. No. 20170051312, incorporated herein by reference, as SEQ ID NOs: 563-679, or a complement thereof, over a segment of at least 8 contiguous nucleotides. For example, one of the two complementary segments of nucleotides of the single-molecule DNA-targeting RNA (or the DNA encoding the segment) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the crRNA sequences set forth in U.S. Pat. App. Pub. No. 20170051312, incorporated herein by reference, as SEQ ID NOs: 563-679, or a complement thereof, over a stretch of at least 8 contiguous nucleotides.

With regard to both a sgRNA and a dgRNA, artificial sequences that share a wide range of identity (approximately at least 50% identity) with naturally occurring tracrRNAs and crRNAs function with Cas9 and dCas9-Fok1 to deliver RNP to target nucleic acids with sequence specificity, particularly provided that the structure of the protein-binding domain of the DNA targeting RNA is conserved. Thus, information and modeling relating to RNA folding and RNA secondary structure of a naturally occurring protein-binding domain of a DNA-targeting RNA provides guidance to design artificial protein-binding domains (either in dgRNA or sgRNA). As a non-limiting example, a functional artificial DNA-targeting RNA may be designed based on the structure of the protein-binding segment of a naturally occurring DNA-targeting segment of an RNA (e.g., including the same or similar number of base pairs along the RNA duplex and including the same or similar “bulge” region as present in the naturally occurring RNA). Structures can readily be produced by one of ordinary skill in the art for any naturally occurring crRNA:tracrRNA pair from any species; thus, in some embodiments an artificial DNA-targeting-RNA is designed to mimic the natural structure for a given species when using the Cas9 (or a related Cas9 or dCas9) from that species. Thus, in some embodiments a suitable DNA-targeting RNA is an artificially designed RNA (non-naturally occurring) comprising a protein-binding domain that was designed to mimic the structure of a protein-binding domain of a naturally occurring DNA-targeting RNA. In exemplary embodiments, the protein-binding segment has a length of from about 10 nucleotides to about 100 nucleotides; e.g., the protein-binding segment has a length of from about 15 nucleotides (nt) to about 80 nt, from about 15 nt to about 50 nt, from about 15 nt to about 40 nt, from about 15 nt to about 30 nt or from about 15 nt to about 25 nt.

Nucleic acids can be analyzed and designed using a variety of computer tools, e.g., Vector NTI (Invitrogen) for nucleic acids and AlignX for comparative sequence analysis of proteins. Further, in silico modeling of RNA structure and folding can be performed using the Vienna RNA package algorithms and RNA secondary structures and folding models can be predicted with RNAfold and RNAcofold, respectively, and visualized with VARNA. See, e.g., Denman (1993), Biotechniques 15, 1090; Hofacker and Stadler (2006), Bioinformatics 22, 1172; and Darty and Ponty (2009), Bioinformatics 25, 1974, each of which is incorporated herein by reference.

Thus, as described herein, in some embodiments, the technology provides methods, systems, kits, compositions, uses, etc. comprising and/or comprising use of a RNP comprising a polypeptide and one or more RNAs. In some embodiments, the RNA comprises a segment (e.g., comprising 6-10 nucleotides, e.g., comprising 6, 7, 8, 9, or 10 nucleotides) that is complementary (e.g., at least 80, 81, 82, 83, 84, 85, 86, 87, 88, 89, 90, 91, 92, 93, 94, 95, 96, 97, 98, 98.5, 99, 99.1, 99.2, 99.3, 99.4, 99.5, 99.6, 99.7, 99.8, 99.9, or 100% complementary) to a nucleotide sequence in the target DNA.

In some embodiments, the RNA comprises a segment comprising a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 431-682 (e.g., SEQ ID NOs: 431-562) of U.S. Pat. App. Pub. No. 20170051312, incorporated herein by reference. In some embodiments, the RNA comprises a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 563-682 of U.S. Pat. App. Pub. No. 20170051312, incorporated herein by reference.

In some embodiments, the polypeptide comprises a segment comprising an amino acid sequence that is at least approximately 75% amino acid identical to amino acids 7-166 or 731-1003 of any of the amino acid sequences set forth as SEQ ID NOs: 1-256 and 795-1346 of U.S. Pat. App. Pub. No. 20170051312, incorporated herein by reference.

### Synthesis and Assembly of RNP and Delivery of RNP

In some embodiments, the fusion protein is synthesized, purified, and assembled in vitro. In some embodiments, the gRNA is transcribed in vitro. In some embodiments, the gRNA is chemically synthesized de novo. In some embodiments, the RNP complex is assembled in vitro using in vitro-transcribed, or de novo-synthesized single guide RNA (sgRNA) and a protein that is synthesized, purified, and folded in vitro.

In some embodiments, an expression system (e.g., comprising an expression vector and a suitable expression host) finds use in producing a polypeptide and/or the RNA of the RNP. Numerous suitable expression vectors are known to those of skill in the art, and many are commercially available. The following vectors are provided by way of example; for eukaryotic host cells; pXT1, pSG5 (Stratagene), pSVK3, pBPV, pMSG, and pSVLSV40 (Pharmacia). However, any other vector may be used so long as it is compatible with the host cell. Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544, incorporated herein by reference).

In some embodiments, the protein is provided as a single polypeptide (e.g., a full gRNA-targeted nuclease (e.g., a dCas9-Fok1 fusion)). In some embodiments, the protein is provided in multiple polypeptides, e.g., a split gRNA-targeted nuclease (e.g., a dCas9-Fok1 fusion) protein provided in two parts, three parts, etc.

In some embodiments, the RNP is provided as a nanoparticle for administration to a live organism.

In some embodiments, the RNP is delivered into cells using a technique or composition related to nucleofection, cell penetrating peptide, viral vesicles, cell surface tunneling protein, ultrasound, electroporation, cell squeezing, nanoparticles, gold or other metal particles, lipid particles, liposomes, viral transduction, viral particles, cell-cell fusion, ballistics, microinjection, and exosome intake.

In some embodiments, the gRNA-targeted nuclease (e.g., a dCas9-Fok1 fusion) comprises a nuclear localization signal (NLS), e.g., an SV40 NLS, to direct the RNP to enter a nucleus. In some embodiments, the protein (e.g., gRNA-targeted nuclease (e.g., a dCas9-Fok1 fusion)) comprises an importin beta binding (IBB) domain sequence, e.g., to promote import of the polypeptide into a cell nucleus, e.g., by an importin (see, e.g., Lott and Cingolani (2011), Biochim Biophys Acta 1813(9): 1578-92, incorporated herein by reference).

In some embodiments, an RNA is introduced into a cell that expresses a gRNA-targeted nuclease (e.g., a dCas9-Fok1 fusion). In some embodiments, crRNA/tracrRNA complexes (e.g., comprising a crRNA and/or a trarcrRNA) are introduced into cells stably expressing a gRNA-targeted nuclease (e.g., a dCas9-Fok1 fusion). In some embodiments, labeled sgRNA is introduced into cells stably expressing a gRNA-targeted nuclease (e.g., a dCas9-Fok1 fusion).

### Nuclease Domains

Embodiments of the technology provide a gRNA-guided nuclease, e.g., comprising a dCas9, homolog, or variant (or DNA-binding domain thereof) and a nuclease (e.g., a Fok1, homolog, or variant thereof). While exemplary embodiments relate to a Fok1 nuclease, the technology is not limited to this nuclease and includes any nuclease that can be provide in the fusion proteins described herein that target nucleic acid rearrangement junctions (e.g., chromosome rearrangement junctions (CRJ), extrachromosomal circle junctions, etc.) and produce double stranded breaks in a chromosome (e.g., by the nuclease activity of the nuclease). In some embodiments, the nuclease portion of the fusion proteins provided herein is or comprises a Type IIS restriction endonuclease or nuclease domain thereof. In some embodiments, the technology comprises use of a catalytically inactive Cas9 fused to Fok1 as described in Guilinger at al. (2014) “Fusion of catalytically inactive Cas9 to Fok1 nuclease improves the specificity of genome modification” Nat Biotechnol 32: 577-82, incorporated herein by reference.

While Fok1 is discussed herein as an exemplary nuclease that finds use in embodiments of the technology, the technology is not limited to protein fusions comprising Fok1. For example, the technology provides gRNA-guided nucleases comprising a Cas9, Cas9-like, or Cas9 homolog (e.g., a dead Cas9, Cas9-like, or Cas9 homolog) fused with other endonucleases (e.g., restriction endonucleases). In some embodiments, the technology provides protein fusions comprising a Cas9, Cas9-like, or Cas9 homolog (e.g., a dead Cas9, Cas9-like, or Cas9 homolog) fused to an endonuclease that produces double stranded breaks comprising blunt ends. Endonucleases that produce blunt ends are known in the art. For example AfeI, AleI, AluI, BmgBI, BsaAI, BsaBI, BsrBI, BstUI, BstZ17I, Cac8I, CviKI, DpnI, DraI, Eco53kI, EcoRV, FspI, HaeIII, HincII, HpaI, Hpy166II, HpyCH4V, MlyI, MscI, MslI, MspA1I, NaeI, NlaIV, NruI, PmeI, PmlI, PshAI, PsiI, PvuII, RsaI, ScaI, SfoI, SmaI, SnaBI, SrfI, SspI, StuI, SwaI, XmnI, and ZraI are known in the art to produce double stranded breaks comprising blunt ends. These and other endonucleases that produce double stranded breaks comprising blunt ends are available from commercial suppliers such as, e.g., New England BioLabs. Restriction endonucleases that produce double stranded breaks comprising sticky ends, frayed ends, blunt ends, etc. are also widely available, e.g., from New England BioLabs and other commercial suppliers.

Blunt end double stranded breaks are repaired in vivo much less efficiently than double stranded breaks comprising an overhang (see, e.g., Costa (1991) “Differences in accumulation of blunt- and cohesive-ended double-strand breaks generated by restriction endonucleases in electroporated CHO cells” Mutat Res 254: 239-246; Suzuki (2010) “Requirement of ATM-dependent pathway for the repair of a subset of DNA double strand breaks created by restriction endonucleases” Genome Integr 1: 4; and Westmoreland (2010) “Blunt-ended DNA double-strand breaks induced by endonucleases PvuII and EcoRV are poor substrates for repair in Saccharomyces cerevisiae” DNA Repair (Amst) 9: 617-626, each of which is incorporated herein by reference. Accordingly, producing blunt end double stranded breaks in a nucleic acid (e.g., in a nucleic acid comprising a nucleic acid rearrangement junction (e.g., chromosome rearrangement junction (CRJ), extrachromosomal circle junction) in a cancer cell) according to the technology provided herein (e.g., by administering a gRNA-guided nuclease (e.g., a protein fusion comprising a dCas9 fused to an endonuclease that produces blunt end double stranded breaks) and a pair of gRNAs targeting a nucleic acid rearrangement junction) provides improved efficiency of killing cancer cells because repair of blunt end double stranded breaks is less efficient in vivo.

Embodiments of the technology comprise use and/or production of a nucleic acid encoding a polypeptide that is an endonuclease (e.g., a restriction endonuclease (e.g., a restriction endonuclease that produces a blunt end double strand break)) or a portion thereof that has endonuclease activity. Embodiments of the technology comprise use and/or production of a nucleic acid encoding a polypeptide that is a gRNA-guided nuclease comprising a Cas9, Cas9-like, or Cas9 homolog (e.g., a dead Cas9, Cas9-like, or Cas9 homolog) fused with an endonuclease (e.g., a restriction endonuclease (e.g., a restriction endonuclease that produces a blunt end double strand break)) or portion thereof that has restriction endonuclease activity (e.g., restriction endonuclease activity that produces a blunt end double strand break).

In some embodiments, the technology comprises use of a “split” protein. For example, in some embodiments, the technology comprises use of a first protein fusion comprising a Cas9, Cas9-like, or Cas9 homolog (e.g., a dead Cas9, Cas9-like, or Cas9 homolog) fused to a first portion of a split protein and a second protein fusion comprising a Cas9, Cas9-like, or Cas9 homolog (e.g., a dead Cas9, Cas9-like, or Cas9 homolog) fused to a second portion of the split protein. According to embodiments of the technology, bringing the two portions of the split protein together (e.g., by a pair of gRNAs) at a target on a nucleic acid (e.g., nucleic acid rearrangement junction (e.g., chromosome rearrangement junction (CRJ), extrachromosomal circle junction)) provides a protein dimer comprising the first portion of the split protein and the second portion of the split protein (e.g., bound to each other), wherein the protein dimer has an activity (e.g., endonuclease activity, conversion of a prodrug to a drug, or other activity that is toxic to a cell such as a cancer cell comprising a nucleic acid rearrangement junction). See, e.g., Wehr (2016) “Split protein biosensor assays in molecular pharmacological studies” Drug Discovery Today 21(3): 415-29; and Ozawa (2006) “Designing split reporter proteins for analytical tools” Analytica Chimica Acta 556(1): 58-68, each of which is incorporated herein by reference. The technology comprises use of any protein that can be split into two parts and reconstituted (e.g., non-covalently) to form a functional protein.

In exemplary embodiments, the technology comprises use of a split horseradish peroxide (HRP) protein where each portion of the split protein has been fused to a Cas9, Cas9-like, or Cas9 homolog (e.g., a dead Cas9, Cas9-like, or Cas9 homolog). In some embodiments, HRP activity converts a prodrug to an active drug at the target site, e.g., specifically at or in a cancer cell (e.g., a cell comprising a nucleic acid comprising a nucleic acid rearrangement junction (e.g., chromosome rearrangement junction (CRJ), extrachromosomal circle junction)). See, e.g., Martell (2016) “A split horseradish peroxidase for the detection of intercellular protein-protein interactions and sensitive visualization of synapses” Nat Biotechnol 34: 774-780; Bonifert (2016) “Recombinant horseradish peroxidase variants for targeted cancer treatment” Cancer Med 5: 1194-1203; Greco (2001) “Horseradish peroxidase-mediated gene therapy: choice of prodrugs in oxic and anoxic tumor conditions” Mol Cancer Ther 1: 151-160; and Tupper (2010) “In vivo characterization of horseradish peroxidase with indole-3-acetic acid and 5-bromoindole-3-acetic acid for gene therapy of cancer” Cancer Gene Ther 17: 420-428, each of which is incorporated herein by reference.

While use of HRP is provided as an example protein that finds use in the present technology, the technology is not limited to use of HRP. For instance, enzymes that find use in the present technology for production of a split protein include, e.g., cytosine deaminase, cytochrome P450, nitroreductase, carboxypeptidase (e.g., carboxypeptidase G2), purine nucleoside phosphorylase, HRP, and carboxylesterase. See, e.g., Malekshah (2016) “Enzyme/Prodrug Systems for Cancer Gene Therapy” Curr Pharmacol Rep 2: 299-308, incorporated herein by reference. Identification, rational design, and production of split proteins is known in the art. See, e.g., Shekhawat (2011) “Split-Protein Systems: Beyond Binary Protein-Protein Interactions” Curr Opin Chem Biol 15(6): 789-97, each of which is incorporated herein by reference. Exemplary split proteins that find use in embodiments of the technology include, e.g., Gal4 (see, e.g., Joung (2000) “A bacterial two-hybrid selection system for studying protein-DNA and protein-protein interactions” PNAS 97(13): 7382-87, incorporated herein by reference), ubiquitin, dihydrofolate reductase (DHFR), focal adhesion kinase, infrared fluorescent protein, green fluorescent protein, beta-lactamase, firefly luciferase, tobacco etch virus protease, chorismite mutase, and thymidine kinase. Computational methods are available to identify where to split a polypeptide (e.g., to identify a first portion of the split protein and a second portion of the split protein). In some embodiments, the “split energy” is used to identify split points and produce split proteins. See, e.g., Dagliyan (2018) “Computational design of chemogenetic and optogenetic split proteins” Nat Commun. 10 02; 9(1): 4042, incorporated herein by reference. In some embodiments, a protein has multiple domains connected by linker sequences such that the portions of the split protein comprise separated domains of the protein.

Embodiments of the technology comprise use and/or production of a first nucleic acid encoding a first polypeptide comprising a Cas9, Cas9-like, or Cas9 homolog (e.g., a dead Cas9, Cas9-like, or Cas9 homolog) fused a first portion of a split protein and a second nucleic acid encoding a second polypeptide comprising a Cas9, Cas9-like, or Cas9 homolog (e.g., a dead Cas9, Cas9-like, or Cas9 homolog) fused a second portion of a split protein.

### DSB Repair Inhibitors

In some embodiments, the technology comprises use of an inhibitor of double-strand break repair (e.g., an inhibitor of DNA-dependent protein kinase (DNA-PK) (e.g., Nu7441)). In some embodiments, the technology comprises use of an inhibitor of the gene product of the human PRKDC (or XRCC7) gene. In some embodiments, the technology comprises use of a DNA-PK inhibitor that is, e.g., NU7441, NU7026, IC86621, IC87361, SU11752, IC486241, caffeine, NK314, CC-115, Compound 401, KU 0060648, LTURM 34, DMNB, ETP 45658, OK-1035, a vanillin, a 6-aryl-2-morpholin-4-yl-4H-pyran-4-one compound, and/or a 6-aryl-2-morpholin-4-yl-4H-thiopyran-4-one compound. In some embodiments, the technology comprises use of a PI3K inhibitor that has inhibitory activity against DNA-PK, e.g., wortmannin, PX-866, PW-458, PI 103 hydrochloride, and/or LY294002. These inhibitors are described in Pospisilova (2017) “Small molecule inhibitors of DNA-PK for tumor sensitization to anticancer therapy” Journal of Physiology and Pharmacology 68: 337; Collins (2004) “The life and death of DNA-PK” Oncogene 24: 949; and Hollick (2003) “2,6-Disubstituted pyran-4-one and thiopyran-4-one inhibitors of DNA-Dependent protein kinase (DNA-PK)” Bioorganic & Medicinal Chemistry Letters 13: 2083, each of which is incorporated herein by reference. In some embodiments, the technology comprises use of an antibody, antibody fragment, aptamer, or other specific binding molecule that inhibits the activity of DNA-PK.

Processes of DSB repair (e.g., through non-homologous end joining) and inhibition of DSB repair are described in Collins (2004) “The life and death of DNA-PK” Oncogene 24: 949, incorporated herein by reference. In some embodiments, molecular targeting strategies are used to inhibit DNA-PK, e.g., siRNA, antisense, and/or microRNA strategies; use of inhibitory peptides, dominant-negative forms of the protein, and/or an inhibitory antibody fragment. In some embodiments, the technology comprises combining use of a gRNA-guided nuclease (e.g., a dCas9-Fok1 fusion) with any inhibitor of the DNA damage response (DDR) pathway. For example, in addition to inhibiting proteins and/or activities of the DSB repair machinery (e.g., DNA-PK, which is targeted by NU7441), the technology comprises inhibiting DNA damage sensor kinases (e.g., ATM and ATR) and cell cycle arrest mediators (e.g., CHK1, CHK2, WEE1, etc.) to improve the toxic effects to tumors of the nucleic acid rearrangement junction-targeting technology provided herein. For example, following induction of a single DSB by CRISPR/Cas9, inhibitors of ATM/ATR or WEE1 resulted in an augmented growth inhibiting effect (see, e.g., van den Berg (2018) “A limited number of double-strand DNA breaks is sufficient to delay cell cycle progression” Nucleic Acids Res 46: 10132-10144, incorporated herein by reference). Further, in some embodiments, the technology comprises combining administering a gRNA-guided nuclease (e.g., dCas9-Fok1 fusion protein) and a composition that decreases the apoptotic threshold of cells, e.g., to increase the toxicity of the technology provided herein. Accordingly, the technology provides combining the nucleic acid rearrangement junction-targeting technology (e.g., dCas9-Fok1 and gRNA pairs) with any drug (at low toxicity levels) that interferes with the sensing, repair, and/or cell cycle arrest following induction of DSBs by the gRNA-guided nuclease. Furthermore, in some embodiments, the technology comprises use of drugs that enhance the toxic effects by modulating the apoptotic machinery.

### Methods

In some embodiments, the technology provides methods for treating cancer. For example, embodiments of methods (e.g., methods for treating cancer) comprise one or more of, e.g., obtaining (e.g., biopsying) a sample from a patient (e.g., a cell and/or tissue from a tumor obtained from a patient having a cancer, suspected of having a cancer, or at risk of having a cancer), sequencing the genome of the sample (e.g., using whole genome sequencing), identifying nucleic acid rearrangement junctions (e.g., chromosome rearrangement junctions (CRJ), extrachromosomal circle junctions, etc.) in the patient tumor sample (e.g., by nucleotide sequence analysis (e.g., by comparison to a normal sample)), designing and/or providing a gRNA pair targeting each nucleic acid rearrangement junction identified (e.g., one or more gRNA pairs each targeting a nucleic acid rearrangement junction); and administering a gRNA-targeted nuclease (e.g., a dCas9-Fok1 fusion) and said gRNA pairs to the patient. In some embodiments, the methods further comprise administering an inhibitor of DSB repair (e.g., an inhibitor of DNA-PK (e.g., Nu7441)) to said patient (e.g., in a non-toxic dose). In some embodiments, the methods comprise administering a DNA-PK inhibitor that is, e.g., NU7441, NU7026, IC86621, IC87361, SU11752, IC486241, caffeine, NK314, CC-115, Compound 401, KU 0060648, LTURM 34, DMNB, ETP 45658, OK-1035, a vanillin, a 6-aryl-2-morpholin-4-yl-4H-pyran-4-one compound, and/or a 6-aryl-2-morpholin-4-yl-4H-thiopyran-4-one compound. In some embodiments, methods comprise administering a PI3K inhibitor that has inhibitory activity against DNA-PK, e.g., wortmannin, PX-866, PW-458, PI 103 hydrochloride, and/or LY294002. In some embodiments, methods comprise administering an antibody, antibody fragment, aptamer, or other specific binding molecule that inhibits the activity of DNA-PK. In some embodiments, methods comprise administering an siRNA, antisense, microRNA, inhibitory peptide, dominant-negative forms of DNA-PK, and/or an inhibitory antibody fragment. In some embodiments, the inhibitor of DSB repair is administered before, substantially simultaneously with, or after the administration of the gRNA-targeted nuclease (e.g., a Cas9-Fok1 fusion) and said gRNA pairs to the patient.

In some embodiments, methods comprise sequencing a partial or full genome of a sample. In some embodiments, methods comprise sequencing a partial or full genome of a cell. In some embodiments, methods comprise producing nucleotide sequences from a sample and comparing the nucleotide sequences with nucleotide sequences from another sample from the same subject (e.g., not comprising a cancer cell) or with sequences obtained from a normal subject, e.g., to identify one or more nucleic acid rearrangement junctions (e.g., chromosome rearrangement junctions (CRJ), extrachromosomal circle junctions, etc.) in the nucleotide sequence from the sample. In some embodiments, methods comprise obtaining 10-1000 bases of sequence comprising a nucleic acid rearrangement junction (e.g., 10, 20, 30, 40, 50, 60, 70, 80, 90, 100, 200, 300, 400, 500, 600, 700, 800, 900, or 1000 bases of sequence comprising a nucleic acid rearrangement junction). In some embodiments, methods comprise obtaining a plurality of sequences (e.g., 1, 2, 3, 4, 5, 6, 7, 8, 9, or 10 sequences and/or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 sequences), each sequence comprising 10-1000 bases of sequence comprising a nucleic acid rearrangement junction (e.g., 10, 20, 30, 40, 50, 60, 70, 80, 90, 100, 200, 300, 400, 500, 600, 700, 800, 900, or 1000 bases of sequence comprising a nucleic acid rearrangement junction).

In some embodiments, identifying nucleic acid rearrangement junctions (e.g., chromosome rearrangement junctions (CRJ), extrachromosomal circle junctions, etc.) comprises identifying 1 to 10 nucleic acid rearrangement junctions (e.g., 1, 2, 3, 4, 5, 6, 7, 8, 9, or 10 nucleic acid rearrangement junctions) from the genome sequence of the sample. In some embodiments, identifying nucleic acid rearrangement junctions comprises identifying 1 to 100 nucleic acid rearrangement junctions (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleic acid rearrangement junctions) from the genome sequence of the sample.

In some embodiments, the technology comprises designing and/or providing a gRNA pair targeting each nucleic acid rearrangement junction identified (e.g., one or more gRNA pairs each targeting a nucleic acid rearrangement junction). For example, in some embodiments, nucleic acid rearrangement junctions are identified from genome sequence information produced by sequencing the genome of the sample (e.g., using whole genome sequencing). Then, in some embodiments, the genome sequence information is used to design two gRNAs for each nucleic acid rearrangement junction that is to be targeted. In some embodiments, 1 to 10 nucleic acid rearrangement junctions are targeted (e.g., 1, 2, 3, 4, 5, 6, 7, 8, 9, or 10 nucleic acid rearrangement junctions are targeted). In some embodiments, 1 to 100 nucleic acid rearrangement junctions are targeted (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleic acid rearrangement junctions are targeted).

In some embodiments, designing two gRNAs comprises identifying nucleotide sequence flanking (e.g., on each side of) a nucleic acid rearrangement junction (e.g., nucleotide sequence on the 5′ side of the nucleic acid rearrangement junction and nucleotide sequence on the 3′ sequence of the nucleic acid rearrangement junction) and designing a gRNA targeting a sequence on each side of the nucleic acid rearrangement junction. In some embodiments, sequence on one side of the nucleic acid rearrangement junction is from one of the two fusion partners joined at the nucleic acid rearrangement junction and sequence on the other side of the nucleic acid rearrangement junction is from a second of the two fusion partners joined at the nucleic acid rearrangement junction. In some embodiments, designing two gRNAs comprises identifying sequence comprising the nucleic acid rearrangement junction and a sequence flanking the nucleic acid rearrangement junction (e.g., within one of the two fusion partners). In some embodiments, designing two gRNAs comprises producing two nucleotide sequences that are complementary to nucleotide sequences flanking (e.g., on each side of) a nucleic acid rearrangement junction (e.g., producing a first nucleotide sequence that is complementary to a nucleotide sequence on the 5′ side of the nucleic acid rearrangement junction and producing a second nucleotide sequence that is complementary to a nucleotide sequence on the 3′ sequence of the nucleic acid rearrangement junction).

In some embodiments, designing two gRNAs comprises producing a nucleotide sequence that is complementary to a nucleotide sequence comprising the nucleic acid rearrangement junction and producing a nucleotide sequence that is complementary to a nucleotide sequence flanking the nucleic acid rearrangement junction (e.g., within one of the two fusion partners). In some embodiments, 1 to 10 pairs of gRNA nucleotide sequences (e.g., 2 to 20 gRNA nucleotide sequences) are produced (e.g., 1, 2, 3, 4, 5, 6, 7, 8, 9, or 10 pairs (e.g., 2, 4, 6, 8, 10, 12, 14, 16, 18, or 20 gRNA nucleotide sequences)). In some embodiments, 1 to 100 pairs of complementary gRNA nucleotide sequences (e.g., 2 to 200 gRNA nucleotide sequences) are produced (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 pairs (e.g., 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, or 200 gRNA nucleotide sequences)).

In some embodiments, the first gRNA and the second gRNA of a gRNA pair are designed to bind approximately 1-1000 bases apart (e.g., 1, 2, 3, 4, 5, 6, 7, 8, 9, 10, 20, 30, 40, 50, 60, 70, 80, 90, 100, 200, 300, 400, 500, 600, 700, 800, 900, or 1000 bases apart) on a nucleic acid (e.g., a chromosome) comprising a nucleic acid rearrangement junction. In some embodiments, the first gRNA and the second gRNA of a gRNA pair are designed to bind approximately 10 to 20 bases (e.g., 13 to 18 bases (e.g., 10, 11, 12, 13, 14, 15, 16, 17, 18, 19, or 20 bases)) apart on a nucleic acid (e.g., a chromosome) comprising a nucleic acid rearrangement junction. In some embodiments, the technology provides the first gRNA and the second gRNA at the target site (e.g., at or near a nucleic acid rearrangement junction) at a distance of 10 to 20 bases (e.g., 13 to 18 bases (e.g., 10, 11, 12, 13, 14, 15, 16, 17, 18, 19, or 20 bases). See, e.g., Tsai (2014) “Dimeric CRISPR RNA-guided FokI nucleases for highly specific genome editing” Nat Biotechnol 32: 569-576, incorporated herein by reference. Data collected during the development of embodiments of the technology described herein indicated that a distance between gRNAs of approximately 11 bases provided adequate and/or robust Fok1 dimerization and nuclease activity.

Methods are known in the art for designing gRNA sequences for targeting genomic sequences. See, e.g., Doench (2014) “Rational design of highly active gRNAs for CRISPR-Cas9-mediated gene inactivation.” Nature biotechnology 32: 1262-67; Doench (2016) “Optimized sgRNA design to maximize activity and minimize off-target effects for genetic screens with CRISPR-Cas9.” Nat Biotechnol 34: 184-94; Radzisheuskaya (2016) “Optimizing sgRNA position markedly improves the efficiency of CRISPR/dCas9-mediated transcriptional repression” Nucleic Acids Research 44: e141-e141; Aach (2014) “Flexible algorithm for identifying specific Cas9 targets in genomes” BioRxiv doi: http://dx.doi.org/10.1101/005074 (2014); Bae (2014) “Cas-OFFinder: a fast and versatile algorithm that searches for potential off-target sites of Cas9 RNA-guided endonucleases” Bioinformatics 30: 1473-1475; Heigwer (2014) “E-CRISP: fast CRISPR target site identification” Nat Methods.11: 122-123; Ma (2013) “A guide RNA sequence design platform for the CRISPR/Cas9 system for model organism genomes” Biomed Res Int 2013 (Article ID 270805); Montague (2014) “CHOPCHOP: a CRISPR/Cas9 and TALEN web tool for genome editing” Nucleic Acids Res. 42(W1): W401-W407; Liu (2015) “CRISPR-ERA: a comprehensive design tool for CRISPR-mediated gene editing, repression and activation” Bioinformatics 31: 3676-78; Ran (2015) “In vivo genome editing using Staphylococcus aureus Cas9” Nature 520: 186-91; Wu (2015) “Target specificity of the CRISPR-Cas9 system” Quant Biol 2: 59-70; Xiao (2014) “CasOT: a genome-wide Cas9/gRNA off-target searching tool” Bioinformatics 30: 1180-82; and Haeussler (2016) “Evaluation of off-target and on-target scoring algorithms and integration into the guide RNA selection tool CRISPOR” Genome Biol 17: 1-12, each of which is incorporated herein by reference. Several online tools are available including from Addgene and the ATUM CRISPR gRNA Design tool.

In some embodiments, methods comprise providing and/or producing gRNA oligonucleotides. In some embodiments, providing and/or producing gRNA oligonucleotides comprises chemical synthesis, ordering from a commercial supplier (e.g., having made RNA oligonucleotides by another for use in the present technology), and other methods known in the art for producing RNA oligonucleotides as described herein.

The technology is not limited in the methodology used to deliver the gRNA-targeted nuclease (e.g., a dCas9-Fok1 fusion) and gRNA pairs to the patient (e.g., to a tumor). Experiments are conducted to evaluate known methods of administration. The following references teach administration of Cas9 RNP and/or gRNA and/or use of plasmid-based expression of Cas9 using nanoparticles, amphiphilic complexes, biomaterials, and other delivery strategies: Lee (2018) “Nanoparticle delivery of CRISPR into the brain rescues a mouse model of fragile X syndrome from exaggerated repetitive behaviours” Nat Biomed Eng 2: 497-507; LaFleur (2019) “A CRISPR-Cas9 delivery system for in vivo screening of genes in the immune system” Nat Commun 10: 1668; Park “In vivo neuronal gene editing via CRISPR-Cas9 amphiphilic nanocomplexes alleviates deficits in mouse models of Alzheimer's disease” Nat Neurosci 22: 524-528; Cicek (2019) “Advances in CRISPR/Cas9 Technology for in Vivo Translation” Biol Pharm Bull 42: 304-311; Sakuma (2014) “Multiplex genome engineering in human cells using all-in-one CRISPR/Cas9 vector system” Sci Rep 4: 5400; and Eoh (2019) “Biomaterials as vectors for the delivery of CRISPR-Cas9” Biomater Sci 7: 1240-1261, each of which is incorporated herein by reference. In some embodiments, experiments are performed to improve administration methods for this technology, e.g., to develop a clinical treatment for cancer therapy. As described herein, normal cells are not affected by this approach because dimerization of the Fok1 endonuclease only occurs at nucleic acid rearrangement junctions in cancer cells. Embodiments of the technology find use in clinical treatment by providing a precise, individualized medicine for treating cancer. The technology is not limited to any particular cancer provided that nucleic acid rearrangement junction are present in cancer cells.

In some embodiments, the methods of treating cancer described herein may include a step of administering a therapeutically effective amount of a pharmaceutical composition comprising a gRNA-targeted nuclease (e.g., a Cas9-Fok1 fusion) as described herein and gRNA pairs targeting one or more nucleic acid rearrangement junctions to the patient and, optionally, an inhibitor of DSB repair. In some embodiments, the gRNA-targeted nuclease (e.g., a Cas9-Fok1 fusion) and gRNA pairs targeting one or more nucleic acid rearrangement junctions to the patient and, optionally, an inhibitor of DSB repair is administered by any suitable route of administration, alone or as part of a pharmaceutical composition. A route of administration may refer to any administration pathway known in the art, including but not limited to aerosol, enteral, nasal, ophthalmic, oral, intracranial, parenteral, rectal, transdermal (e.g., topical cream or ointment, patch), or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal.

The term “effective amount” as used herein refers to an amount of a composition (e.g., a gRNA-targeted nuclease (e.g., a Cas9-Fok1 fusion) and gRNA pairs targeting one or more nucleic acid rearrangement junctions) that produces a desired effect. For example, a population of cells may be contacted with an effective amount of a composition to study its effect in vitro (e.g., cell culture) or to produce a desired therapeutic effect ex vivo or in vitro. An effective amount of a composition may be used to produce a therapeutic effect in a subject, such as preventing or treating a target condition (e.g., cancer), alleviating symptoms associated with the condition (e.g., cancer), or producing a desired physiological effect. In such a case, the effective amount of a composition is a “therapeutically effective amount,” “therapeutically effective concentration”, or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the composition (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further, an effective or therapeutically effective amount may vary depending on whether the composition is administered alone or in combination with another compound, drug, therapy or other therapeutic method or modality (e.g., an inhibitor of DSB repair). One skilled in the clinical and pharmacological arts will be able to determine an effective amount or therapeutically effective amount through routine experimentation, namely by monitoring a cell's or subject's response to administration of a compound and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, incorporated herein by reference.

“Treating” or “treatment” of a condition (e.g., cancer) may refer to preventing the condition (e.g., cancer), slowing the onset or rate of development of the condition (e.g., cancer), reducing the risk of developing the condition (e.g., cancer), preventing or delaying the development of symptoms associated with the condition (e.g., cancer), reducing or ending symptoms associated with the condition (e.g., cancer), generating a complete or partial regression of the condition (e.g., cancer), or some combination thereof. Treatment may also mean a prophylactic or preventative treatment of a condition (e.g., cancer).

As used herein, a “subject in need of treatment” is a subject having a disorder (e.g., cancer) caused by one or more nucleic acid rearrangement junctions or a subject having an increased risk of developing such a disorder relative to the population at large. In some embodiments, a subject in need of treatment has a precancerous condition. In some embodiments, a subject in need of treatment has cancer. A “subject” includes a mammal. The mammal can be, e.g., any mammal, e.g., a human, primate, bird, mouse, rat, fowl, dog, cat, cow, horse, goat, camel, sheep, or a pig. Preferably, the mammal is a human. In some embodiments, the subject is a human subject who has been diagnosed with, has symptoms of, or is at risk of developing a cancer or a precancerous condition.

In some embodiments, a subject in need of treatment is a subject having a disorder (e.g., cancer) associated with, indicated by, and/or caused by one or more nucleic acid rearrangement junctions. In some embodiments, a subject in need of treatment has a precancerous condition associated with, indicated by, and/or caused by one or more nucleic acid rearrangement junctions. In some embodiments, a subject in need of treatment has cancer associated with, indicated by, and/or caused by one or more nucleic acid rearrangement junctions. In some embodiments, a subject in need of treatment has one or more cancers selected from the group consisting of brain and central nervous system (CNS) cancer, head and neck cancer, kidney cancer, ovarian cancer, pancreatic cancer, leukemia, lung cancer, lymphoma, myeloma, sarcoma, breast cancer, prostate cancer and a hematological cancer. In some embodiments, a subject in need of treatment has a hematologic cancer, wherein the hematologic cancer is leukemia or lymphoma. One exemplary leukemia is MLL. In some embodiments, the cancer is a multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

In some embodiments, a subject in need of treatment has been previously diagnosed or identified as having cancer or a precancerous condition. In some embodiments, a subject in need of treatment has (is suffering from) cancer or a precancerous condition. Alternatively, a subject in need of treatment has an increased risk of developing such disorder relative to the population at large (e.g., a subject who is predisposed to developing such disorder relative to the population at large).

In some embodiments, a subject in need of treatment has a cancer associated with one or more nucleic acid rearrangement junctions. In some embodiments, a subject in need of treatment may have increased mRNA, protein, and/or activity level of at least one gene product encoded by a nucleotide sequence that forms a nucleic acid rearrangement junction. As used herein, the term “increase in activity” refers to increased or a gain of function of a gene product/protein compared to the wild type. Accordingly, an increase in mRNA or protein expression and/or activity levels can be detected using any suitable method available in the art.

In some embodiments, a subject in need of treatment has already undergone, is undergoing, or will undergo at least one therapeutic intervention for the cancer or precancerous condition.

In some embodiments, a subject in need of treatment may have refractory cancer on most recent therapy. “Refractory cancer” means cancer that does not respond to treatment. The cancer may be resistant at the beginning of treatment or it may become resistant during treatment. Refractory cancer is also called resistant cancer. In some embodiments, the subject in need of treatment has cancer recurrence following remission on most recent therapy. In some embodiments, the subject in need of therapy received and failed all known effective therapies for cancer treatment. In some embodiments, the subject in need of treatment received at least one prior therapy.

In some embodiments, a subject in need of treatment may have a secondary cancer as a result of a previous therapy. “Secondary cancer” means cancer that arises due to or as a result from previous carcinogenic therapies, such as chemotherapy.

The present technology provides personalized medicine, treatment, and/or cancer management for a subject by genetic screening to detect one or more nucleic acid rearrangement junctions in the subject. For example, the present technology provides methods for treating or alleviating a symptom of cancer or a precancerous condition in a subject in need of treatment by determining a genotype and/or genome sequence of a subject (e.g., sequencing the genome of one or more cancer cells, cancer tissues, and/or cancer samples from the subject), identifying nucleic acid rearrangement junctions for targeting (e.g., nucleic acid rearrangement junctions that are statistically more prominent for the cancer cells from the subject in need of treatment and/or unique to the cancer cells from the subject in need of treatment relative to normal cells (e.g., normal cells from the subject in need of treatment (e.g., from a non-cancerous cell or tissue) and/or from a subject who does not have a cancer and/or who does not have the same cancer as the subject in need of treatment)), and administering to the subject a composition of the technology (e.g., a gRNA-targeted nuclease (e.g., a Cas9-Fok1 fusion) and gRNA pairs targeting one or more nucleic acid rearrangement junctions).

By “sample” it means any biological sample derived from the subject, includes but is not limited to, cells, tissues samples, body fluids (including, but not limited to, mucus, blood, plasma, serum, urine, saliva, and semen), tumor cells, and tumor tissues. In some embodiments, the sample is selected from bone marrow, peripheral blood cells, blood, plasma, and serum. Samples can be provided by the subject under treatment or testing. Alternatively samples can be obtained by the physician according to routine practice in the art.

The compositions and methods provided herein may be used for the treatment of a wide variety of cancers including tumors such as prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, the compositions provided herein can be used to treat the following types of cancers: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

In some embodiments, treating cancer results in a reduction in size of a tumor. A reduction in size of a tumor may also be referred to as “tumor regression”. In some embodiments, after treatment, tumor size is reduced by 5% or greater relative to its size prior to treatment; in some embodiments, tumor size is reduced by 10% or greater; in some embodiments, tumor size is reduced by 20% or greater; in some embodiments, tumor size is reduced by 30% or greater; in some embodiments, tumor size is reduced by 40% or greater; and, in some embodiments, tumor size is reduced by 50% or greater; and in some embodiments, tumor size is reduced by greater than 75% or greater. Size of a tumor may be measured by any reproducible means of measurement. The size of a tumor may be measured as a diameter of the tumor.

In some embodiments, treating cancer results in a reduction in tumor volume. In some embodiments, after treatment, tumor volume is reduced by 5% or greater relative to its size prior to treatment; in some embodiments, tumor volume is reduced by 10% or greater; in some embodiments, tumor volume is reduced by 20% or greater; in some embodiments, tumor volume is reduced by 30% or greater; in some embodiments, tumor volume is reduced by 40% or greater; and, in some embodiments, tumor volume is reduced by 50% or greater; in some embodiments, tumor volume is reduced by greater than 75% or greater. Tumor volume may be measured by any reproducible means of measurement.

In some embodiments, treating cancer results in a decrease in number of tumors. In some embodiments, after treatment, tumor number is reduced by 5% or greater relative to number prior to treatment; in some embodiments, tumor number is reduced by 10% or greater; in some embodiments, tumor number is reduced by 20% or greater; in some embodiments, tumor number is reduced by 30% or greater; in some embodiments, tumor number is reduced by 40% or greater; and, in some embodiments, tumor number is reduced by 50% or greater; in some embodiments, tumor number is reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

In some embodiments, treating results in a decrease in number of metastatic lesions in other tissues or organs distant from the primary tumor site. In some embodiments, after treatment, the number of metastatic lesions is reduced by 5% or greater relative to number prior to treatment; in some embodiments, the number of metastatic lesions is reduced by 10% or greater; in some embodiments, the number of metastatic lesions is reduced by 20% or greater; in some embodiments, the number of metastatic lesions is reduced by 30% or greater; in some embodiments, the number of metastatic lesions is reduced by 40% or greater; and, in some embodiments, the number of metastatic lesions is reduced by 50% or greater; and, in some embodiments, the number of metastatic lesions is reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

In some embodiments, treating cancer results in an increase in average survival time of a population of treated subjects in comparison to a population receiving carrier alone. In some embodiments, the average survival time is increased by more than 30 days; in some embodiments, the average survival time is increased by more than 60 days; in some embodiments, the average survival time is increased by more than 90 days; and, in some embodiments, the average survival time is increased by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

In some embodiments, treating cancer results in an increase in average survival time of a population of treated subjects in comparison to a population of untreated subjects. Preferably, the average survival time is increased by more than 30 days; in some embodiments, the average survival time is increased by more than 60 days; in some embodiments, the average survival time is increased by more than 90 days; and, in some embodiments, the average survival time is increased by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

In some embodiments, treating cancer results in increase in average survival time of a population of treated subjects in comparison to a population receiving monotherapy with a composition that is not a composition of the present technology. In some embodiments, the average survival time is increased by more than 30 days; in some embodiments, the average survival time is increased by more than 60 days; in some embodiments, the average survival time is increased by more than 90 days; and, in some embodiments the average survival time is increased by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

In some embodiments, treating cancer results in a decrease in the mortality rate of a population of treated subjects in comparison to a population receiving carrier alone. In some embodiments, treating cancer results in a decrease in the mortality rate of a population of treated subjects in comparison to an untreated population. In some embodiments, treating cancer results in a decrease in the mortality rate of a population of treated subjects in comparison to a population receiving monotherapy with a composition that is not a composition of the present technology. In some embodiments, the mortality rate is decreased by more than 2%; in some embodiments, the mortality rate is decreased by more than 5%; in some embodiments, the mortality rate is decreased by more than 10%; and, in some embodiments, the mortality rate is decreased by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

In some embodiments, treating cancer results in a decrease in tumor growth rate. In some embodiments, after treatment, tumor growth rate is reduced by at least 5% relative to growth rate prior to treatment; in some embodiments, tumor growth rate is reduced by at least 10%; in some embodiments, tumor growth rate is reduced by at least 20%; in some embodiments, tumor growth rate is reduced by at least 30%; in some embodiments, tumor growth rate is reduced by at least 40%; in some embodiments, tumor growth rate is reduced by at least 50%; and, in some embodiments, tumor growth rate is reduced by at least 50%; and in some embodiments, tumor growth rate is reduced by at least 75%. Tumor growth rate may be measured by any reproducible means of measurement. Tumor growth rate can be measured according to a change in tumor diameter per unit time.

In some embodiments, treating cancer results in a decrease in tumor regrowth. In some embodiments, after treatment, tumor regrowth is less than 5%; in some embodiments, tumor regrowth is less than 10%; in some embodiments, tumor regrowth is less than 20%; in some embodiments, tumor regrowth is less than 30%; in some embodiments, tumor regrowth is less than 40%; in some embodiments, tumor regrowth is less than 50%; and, in some embodiments, tumor regrowth is less than 50%; and, in some embodiments, tumor regrowth is less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

In some embodiments, treating or preventing a cell proliferative disorder results in a reduction in the rate of cellular proliferation. In some embodiments, after treatment, the rate of cellular proliferation is reduced by at least 5%; in some embodiments, the rate of cellular proliferation is reduced by at least 10%; in some embodiments, the rate of cellular proliferation is reduced by at least 20%; in some embodiments, the rate of cellular proliferation is reduced by at least 30%; in some embodiments, the rate of cellular proliferation is reduced by at least 40%; in some embodiments, the rate of cellular proliferation is reduced by at least 50%; and, in some embodiments, the rate of cellular proliferation is reduced by at least 50%; and in some embodiments, the rate of cellular proliferation is reduced by at least 75%. The rate of cellular proliferation may be measured by any reproducible means of measurement. The rate of cellular proliferation is measured, for example, by measuring the number of dividing cells in a tissue sample per unit time.

In some embodiments, treating or preventing a cell proliferative disorder results in a reduction in the proportion of proliferating cells. In some embodiments, after treatment, reduced by at least 5%; in some embodiments, by at least 10%; in some embodiments, the proportion of proliferating cells is reduced by at least 20%; in some embodiments, the proportion of proliferating cells is reduced by at least 30%; in some embodiments, the proportion of proliferating cells is reduced by at least 40%; in some embodiments, the proportion of proliferating cells is reduced by at least 50%; and, in some embodiments, the proportion of proliferating cells is reduced by at least 50%; and in some embodiments, the proportion of proliferating cells is reduced by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

In some embodiments, treating or preventing a cell proliferative disorder results in a decrease in size of an area or zone of cellular proliferation. In some embodiments, after treatment, the size of an area or zone of cellular proliferation is reduced by at least 5% relative to its size prior to treatment; in some embodiments, the size of an area or zone of cellular proliferation is reduced by at least 10%; in some embodiments, the size of an area or zone of cellular proliferation is reduced by at least 20%; in some embodiments, the size of an area or zone of cellular proliferation is reduced by at least 30%; in some embodiments, the size of an area or zone of cellular proliferation is reduced by at least 40%; in some embodiments, the size of an area or zone of cellular proliferation is reduced by at least 50%; and, in some embodiments, the size of an area or zone of cellular proliferation is reduced by at least 50%; and in some embodiments, the size of an area or zone of cellular proliferation is reduced by at least 75%. Size of an area or zone of cellular proliferation may be measured by any reproducible means of measurement. The size of an area or zone of cellular proliferation may be measured as a diameter or width of an area or zone of cellular proliferation.

In some embodiments, treating or preventing a cell proliferative disorder results in a decrease in the number or proportion of cells having an abnormal appearance or morphology. In some embodiments, after treatment, the number of cells having an abnormal morphology is reduced by at least 5% relative to its size prior to treatment; in some embodiments, the number of cells having an abnormal morphology is reduced by at least 10%; in some embodiments, the number of cells having an abnormal morphology is reduced by at least 20%; in some embodiments, the number of cells having an abnormal morphology is reduced by at least 30%; in some embodiments, the number of cells having an abnormal morphology is reduced by at least 40%; in some embodiments, the number of cells having an abnormal morphology is reduced by at least 50%; and, in some embodiments, the number of cells having an abnormal morphology is reduced by at least 50%; and, in some embodiments, the number of cells having an abnormal morphology is reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. For example, according to embodiments of the technology provided herein, a composition described herein acts selectively on a cancer or precancerous cell but not on a normal cell. In some embodiments, an event occurs selectively in population A relative to population B if it occurs greater than two times more frequently in population A as compared to population B. In some embodiments, an event occurs selectively in population A relative to population B if it occurs greater than five times more frequently in population A as compared to population B. In some embodiments, an event occurs selectively in population A relative to population B if it occurs greater than ten times more frequently in population A as compared to population B. In some embodiments, an event occurs selectively in population A relative to population B if it occurs greater than fifty times more frequently in population A as compared to population B. In some embodiments, an event occurs selectively in population A relative to population B if it occurs greater than one hundred times more frequently in population A as compared to population B. In some embodiments, an event occurs selectively in population A relative to population B if it occurs greater than one thousand times more frequently in population A as compared to population B. For example, cell death (e.g., resulting from producing DSBs according to the technology provided herein) would be said to occur selectively in cancer cells if it occurred greater than twice as frequently in cancer cells as compared to normal cells.

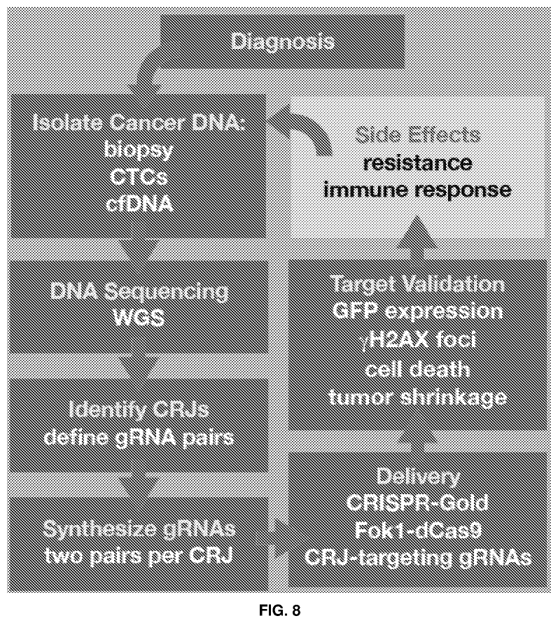

As described herein, some embodiments of the technology provide a precision method for treating a cancer. In some embodiments, methods comprise performing whole genome sequencing (WGS) on isolated cancer DNA. In some embodiments, methods comprise identifying nucleic acid rearrangement junctions (e.g., computationally) and synthesizing pairs of nucleic acid rearrangement junction-targeting gRNAs. In some embodiments, methods comprise delivering the gRNAs and the Fok1-dCas9 CRISPR reagents to a tumor. In some embodiments, methods comprise assessing the efficiency of delivery and tumor control. See, e.g., FIG. 8.

### Reaction Mixtures

In some embodiments, the technology provides a reaction mixture. In some embodiments, the technology provides a reaction mixture comprising: a) a nucleic acid (e.g., a chromosome) comprising a nucleic acid rearrangement junction; b) a first gRNA-guided nuclease (e.g., a dCas9-Fok1 fusion); c) a first gRNA; d) a second gRNA-guided nuclease (e.g., a dCas9-Fok1 fusion); and e) a second gRNA-guided nuclease. In some embodiments, the first gRNA-guided nuclease (e.g., dCas9-Fok1 fusion) binds the first gRNA. In some embodiments, the second gRNA-guided nuclease (e.g., a dCas9-Fok1 fusion) binds the second gRNA. In some embodiments, the first gRNA-guided nuclease (e.g., dCas9-Fok1 fusion) binding the first gRNA is bound to the nucleic acid comprising the nucleic acid rearrangement junction. In some embodiments, the second gRNA-guided nuclease (e.g., dCas9-Fok1 fusion) binding the second gRNA is bound to the nucleic acid comprising the nucleic acid rearrangement junction. In some embodiments, the first gRNA is bound (e.g., hybridized to) the nucleic acid comprising the nucleic acid rearrangement junction. In some embodiments, the second gRNA is bound (e.g., hybridized to) the nucleic acid comprising the nucleic acid rearrangement junction.

In some embodiments, the first gRNA-guided nuclease (e.g., dCas9-Fok1 fusion) binding the first gRNA is bound to the nucleic acid comprising the nucleic acid rearrangement junction and the second gRNA-guided nuclease (e.g., dCas9-Fok1 fusion) binding the second gRNA is bound to the nucleic acid; and the first gRNA-guided nuclease (e.g., dCas9-Fok1 fusion) binding the first gRNA and the second gRNA-guided nuclease (e.g., dCas9-Fok1 fusion) binding the second gRNA flank the nucleic acid rearrangement junction (e.g., approximately 1-100 nt (e.g., 1, 2, 3, 4, 5, 6, 7, 8, 9, 10, 20, 30, 40, 50, 60, 70, 80, 90, or 100 bases or nt) are between the first gRNA-guided nuclease (e.g., dCas9-Fok1 fusion) binding the first gRNA and the second gRNA-guided nuclease (e.g., dCas9-Fok1 fusion) binding the second gRNA. In some embodiments, the first gRNA-guided nuclease (e.g., dCas9-Fok1 fusion) binding the first gRNA is bound to the nucleic acid comprising the nucleic acid rearrangement junction and the second gRNA-guided nuclease (e.g., dCas9-Fok1 fusion) binding the second gRNA is bound to the nucleic acid; and the first gRNA-guided nuclease (e.g., dCas9-Fok1 fusion) binding the first gRNA is bound to the nucleic acid rearrangement junction and the second gRNA-guided nuclease (e.g., dCas9-Fok1 fusion) binding the second gRNA is adjacent (e.g., within 1-100 nt (e.g., 1, 2, 3, 4, 5, 6, 7, 8, 9, 10, 20, 30, 40, 50, 60, 70, 80, 90, or 100 bases or nt) to the nucleic acid rearrangement junction.

In some embodiments, the first gRNA-guided nuclease (e.g., dCas9-Fok1 fusion) binding the first gRNA is bound to the nucleic acid comprising the nucleic acid rearrangement junction and the second gRNA-guided nuclease (e.g., dCas9-Fok1 fusion) binding the second gRNA is bound to the nucleic acid; and the first gRNA-guided nuclease (e.g., dCas9-Fok1 fusion) binding the first gRNA and the second gRNA-guided nuclease (e.g., dCas9-Fok1 fusion) binding the second gRNA flank the nucleic acid rearrangement junction (e.g., approximately 10 to 20 bases or nt (e.g., 13 to 18 bases (e.g., 10, 11, 12, 13, 14, 15, 16, 17, 18, 19, or 20 bases))) are between the first gRNA-guided nuclease (e.g., dCas9-Fok1 fusion) binding the first gRNA and the second gRNA-guided nuclease (e.g., dCas9-Fok1 fusion) binding the second gRNA. In some embodiments, the first gRNA-guided nuclease (e.g., dCas9-Fok1 fusion) binding the first gRNA is bound to the nucleic acid comprising the nucleic acid rearrangement junction and the second gRNA-guided nuclease (e.g., dCas9-Fok1 fusion) binding the second gRNA is bound to the nucleic acid; and the first gRNA-guided nuclease (e.g., dCas9-Fok1 fusion) binding the first gRNA is bound to the nucleic acid rearrangement junction and the second gRNA-guided nuclease (e.g., dCas9-Fok1 fusion) binding the second gRNA is adjacent (e.g., within approximately 10 to 20 bases or nt (e.g., 13 to 18 bases or nt (e.g., 10, 11, 12, 13, 14, 15, 16, 17, 18, 19, or 20 bases or nt))) to the nucleic acid rearrangement junction.

In some embodiments, the first gRNA-guided nuclease (e.g., dCas9-Fok1 fusion) binding the first gRNA and the second gRNA-guided nuclease (e.g., dCas9-Fok1 fusion) binding the second gRNA form a dimer of proteins or protein domains (e.g., a dimer of Fok1 proteins or Fek1 domains) that has nuclease activity, e.g., to produce a double strand break in a nucleic acid (e.g., in a chromosome comprising a nucleic acid rearrangement junction). In some embodiments, the first gRNA and the second gRNA bind to a nucleic acid (e.g., comprising a nucleic acid rearrangement junction) at locations and/or a distance from each other that promote the dimerization of the nuclease domains (e.g., the Fok1 domains) of two gRNA-guided nucleases (e.g., two dCas9-Fok1 fusions), e.g., to produce a dimer that has nuclease activity, e.g., to produce a double stranded break in a nucleic acid comprising a nucleic acid rearrangement junction.

In some embodiments, the nucleic acid comprising the nucleic acid rearrangement junction comprises a double stranded break (e.g., between the first gRNA-guided nuclease (e.g., dCas9-Fok1 fusion) binding the first gRNA and the second gRNA-guided nuclease (e.g., dCas9-Fok1 fusion) binding the second gRNA (e.g., between the first and second gRNAs bound to the nucleic acid comprising the nucleic acid rearrangement junction).

In some embodiments, reaction mixtures comprise 1 to 10 nucleic acids each comprising a nucleic acid rearrangement junction (e.g., 1, 2, 3, 4, 5, 6, 7, 8, 9, or 10 nucleic acids each comprising a nucleic acid rearrangement junction). In some embodiments, reaction mixtures comprise 1 to 100 nucleic acids each comprising a nucleic acid rearrangement junction (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleic acids each comprising a nucleic acid rearrangement junction). Accordingly, embodiments provide reaction mixtures comprising a plurality of nucleic acids each comprising a nucleic acid rearrangement junction; and a plurality of gRNA pairs, wherein each gRNA pair is specific for a nucleic acid comprising a nucleic acid rearrangement junction (e.g., each pair comprises a first gRNA complementary to the nucleic acid comprising a nucleic acid rearrangement junction and a second gRNA complementary to the nucleic acid comprising the nucleic acid rearrangement junction, wherein the first and second gRNAs are complementary to a first and second nucleotide sequences flanking the nucleic acid rearrangement junction or one gRNA is complementary to a sequence comprising the nucleic acid rearrangement junction and the second gRNA is complementary to a nucleotide sequence adjacent to the CJR). Further, embodiments provide a plurality of gRNA-guided nucleases (e.g., dCas9-Fok1 fusions) bound to gRNAs and bound to a plurality of nucleic acids each comprising a nucleic acid rearrangement junction. Similarly, embodiments provide a plurality of gRNA pairs bound to a plurality of nucleic acids each comprising a nucleic acid rearrangement junction. In some embodiments, the gRNA pairs flank the nucleic acid rearrangement junction. In some embodiments, the gRNA pairs comprise a gRNA bound to the nucleic acid rearrangement junction and a gRNA bound adjacent to the nucleic acid rearrangement junction. That is, in some embodiments, the first and second gRNAs are complementary to a first and second nucleotide sequences flanking the nucleic acid rearrangement junction or one gRNA is complementary to a sequence comprising the nucleic acid rearrangement junction and the second gRNA is complementary to a nucleotide sequence adjacent to the CJR. In any of the above reaction mixture embodiments, the reaction mixtures may further comprise an inhibitor of DSB repair (e.g., an inhibitor of DNA-PK (e.g., Nu7441)).

### Kits

In some embodiments, the technology is related to kits, e.g., kits for treating cancer. In some embodiments, kits comprise a composition as described herein. In some embodiments, kits comprise a gRNA-targeted nuclease (e.g., a dCas9-nuclease (e.g., Fok1) fusion). In some embodiments, kits comprise an inhibitor of DSB repair (e.g., an inhibitor of DNA-PK as described herein (e.g., Nu7441 or another inhibitor of DNA-PK)). In some embodiments, kits comprise a gRNA-targeted nuclease (e.g., a dCas9-nuclease (e.g., Fok1) fusion) and an inhibitor of DSB repair (e.g., an inhibitor of DNA-PK as described herein (e.g., Nu7441 or another inhibitor of DNA-PK)). In some embodiments, kits comprise a solution for preparing a solution for administering one or more kit components to a subject (e.g., for preparing a solution of a gRNA-targeted nuclease (e.g., a dCas9-nuclease (e.g., Fok1) fusion), an inhibitor of DSB repair (e.g., an inhibitor of DNA-PK as described herein (e.g., Nu7441 or another inhibitor of DNA-PK)), and one or more pairs of gRNAs). In some embodiments, kits further comprise computer-readable media comprising software tools for analyzing genomic sequence, identifying nucleic acid rearrangement junctions, designing gRNA (e.g., designing pairs of gRNAs for each nucleic acid rearrangement junction that is targeted), and producing gRNA. In some embodiments, kits comprise a means for administration of the gRNA-targeted nuclease and gRNAs.

In some embodiments, a kit further includes one or more additional reagents, where such additional reagents can be selected from: a buffer; a wash buffer; a control reagent; a control expression vector or RNA polynucleotide; a reagent for in vitro production of the gRNA-targeted nuclease (e.g., a dCas9-Fok1 fusion) from DNA; and the like. In some embodiments, the fusion protein further comprises a domain providing enhanced or improved localization (e.g., transport) to the nucleus (e.g., an NLS, an IBB, etc.) In some embodiments, components of the kit are in separate containers; in some embodiments, one or more components of a kit are combined in a single container. Further, in some embodiments, a kit can further include instructions for using the components of the kit to practice a method described herein. In some embodiments, kits comprise one or more compositions as described herein, e.g., packaged in one or more containers for use by a user. Further, in some embodiments, a kit can further include instructions for using the components of the kit to practice a method described herein.

In some embodiments of kits, kits comprise one or more vectors. In some embodiments of the technology, the gRNA-targeted nuclease (e.g., a dCas9-Fok1 fusion) is codon optimized for expression by the desired cell type, preferentially a eukaryotic cell, preferably a mammalian cell or a human cell.

In some embodiments, kits comprise packaging cells that are used to form virus particles that are capable of infecting a host cell. Such cells include 293 cells, which package adenovirus, and psi2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, hut lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

The present disclosure provides kits for carrying out a subject method. In some embodiments, a kit comprises a gRNA-targeted nuclease (e.g., a dCas9-Fok1 fusion) and/or a nucleic acid having nucleotides encoding a gRNA-targeted nuclease (e.g., a dCas9-Fok1 fusion). In some embodiments, a kit can further include one or more additional reagents, where such additional reagents can be selected from: a dilution buffer; a reconstitution solution; a wash buffer; a control reagent; a control expression vector or RNA polynucleotide; a reagent for in vitro production of gRNA-targeted nuclease (e.g., a dCas9-Fok1 fusion) from DNA, and the like. The components of a subject kit can be in the same or different containers (in any desired combination).

In addition to above-mentioned components, a kit can further include instructions for using the components of the kit to practice a method as described herein. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or sub-packaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Systems

In some embodiments, the technology provides systems, e.g., for treating cancer. In some embodiments, systems comprise a composition as described herein. In some embodiments, systems comprise a gRNA-targeted nuclease (e.g., a dCas9-nuclease (e.g., Fok1) fusion). In some embodiments, systems comprise an inhibitor of DSB repair (e.g., an inhibitor of DNA-PK as described herein (e.g., Nu7441 or another inhibitor of DNA-PK)). In some embodiments, systems comprise a gRNA-targeted nuclease (e.g., a dCas9-nuclease (e.g., Fok1) fusion) and an inhibitor of DSB repair (e.g., an inhibitor of DNA-PK as described herein (e.g., Nu7441 or another inhibitor of DNA-PK)). In some embodiments, systems comprise a solution for preparing a solution for administering one or more kit components to a subject (e.g., for preparing a solution of a gRNA-targeted nuclease (e.g., a dCas9-nuclease (e.g., Fok1) fusion), an inhibitor of DSB repair (e.g., an inhibitor of DNA-PK as described herein (e.g., Nu7441 or another inhibitor of DNA-PK)), and one or more pairs of gRNAs).

In some embodiments, systems comprise a sequencer configured to produce a nucleotide sequence from a nucleic acid (e.g., from a nucleic acid in a sample obtained from a subject in need of treatment). For example, in some embodiments, sequencing is utilized to provide an analysis of the sequence and frequency of nucleic acid rearrangement junctions in samples. Illustrative non-limiting examples of nucleic acid sequencing techniques implemented on a sequencer in embodiments of the technology include, but are not limited to, chain terminator (Sanger) sequencing, dye terminator sequencing, and high-throughput sequencing methods. Many of these sequencing methods are well known in the art, See, e.g., Sanger (1997) Proc. Natl. Acad. Sci. USA 74: 5463-5467; Maxam (1977) Proc. Natl. Acad. Sci. USA 74: 560-564; Drmanac (1998) Nat. Biotechnol. 16: 54-58; Kato (2009) Int. J. Clin. Exp. Med. 2: 193-202; Ronaghi (1996) Anal. Biochem. 242: 84-89; Margulies (2005) Nature 437: 376-380; Ruparel (2005) Proc. Natl. Acad. Sci. USA 102: 5932-5937; Harris (2008) Science 320: 106-109; Levene (2003) Science 299: 682-686; Korlach (2008) Proc. Natl. Acad. Sci. USA 105: 1176-1181; Branton (2008) Nat. Biotechnol. 26(10): 1146-53; and Eid (2009) Science 323: 133-138, each of which is herein incorporated by reference. In some embodiments, “four-color sequencing by synthesis using cleavable fluorescent nucleotide reversible terminators” is used, e.g., as commercialized by Intelligent Bio-Systems. The technology is described in Turro (2006) PNAS 103: 19635-40; and in U.S. Pat. Appl. Pub. Nos. 2010/0323350, 2010/0063743, 2010/0159531, 20100035253, 20100152050, each of which is incorporated herein by reference. In some embodiments, nanopore sequencing is used in which integrated circuits enable massively parallel single-molecule DNA sequencing. See, e.g., Rothberg (2011) “An integrated semiconductor device enabling non-optical genome sequencing” Nature 475: 348; Timp (2010) “Nanopore Sequencing—Electrical Measurements of the Code of Life”, IEEE Transactions on Nanotechnology 9: 281; and Stoddart (2009) “Single-nucleotide discrimination in immobilized DNA oligonucleotides with a biological nanopore” PNAS 106: 7703, each of which is incorporated herein by reference. Morozova and Marra (2008) provide a review of some such technologies in Genomics 92: 255, which is incorporated herein by reference; additional discussions are found in Mardis (2008) Annu. Rev. Genomics Hum. Genet. 9: 387-402; and in Fuller (2009) Nat. Biotechnol. 27: 1013, each of which is incorporated herein by reference. Several sequencing technologies are commercially available, e.g., sequencing techniques such as single molecule real time sequencing (Pacific Biosciences), sequencing by synthesis (Illumina, Inc.), 454 pyrosequencing (Roche Diagnostics, Inc.), SOLiD sequencing (Life Technologies, Inc.), and ion semiconductor sequencing (Life Technologies, Inc.)

In some embodiments, systems comprise one or more vectors. In some system embodiments of the technology, the gRNA-targeted nuclease (e.g., a dCas9-Fok1 fusion) is codon optimized for expression by the desired cell type, preferentially a eukaryotic cell, preferably a mammalian cell or a human cell.

In some embodiments, systems comprise packaging cells used to form virus particles that are capable of infecting a host cell. Such cells include 293 cells, which package adenovirus, and psi2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, hut lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

Some embodiments comprise a computer system upon which embodiments of the present teachings may be implemented. For example, in some embodiments, systems further comprise software tools for analyzing genomic sequence and identifying nucleic acid rearrangement junctions in nucleotide sequences. In various embodiments, a computer system includes a bus or other communication mechanism for communicating information, and a processor coupled with the bus for processing information. In various embodiments, the computer system includes a memory, which can be a random access memory (RAM) or other dynamic storage device, coupled to the bus for identifying bases (e.g., making “base calls”), and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display. This input device typically has two degrees of freedom in two axes, a first axis (e.g., x) and a second axis (e.g., y), that allows the device to specify positions in a plane.

A computer system can perform embodiments of the present technology. Consistent with certain implementations of the present teachings, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present teachings are not limited to any specific combination of hardware circuitry and software.

The term “computer-readable medium” as used herein refers to any media that participates in providing instructions to the processor for execution. Such a medium can take many forms, including but not limited to, non-volatile media, volatile media, and transmission media. Examples of non-volatile media can include, but are not limited to, optical or magnetic disks, such as a storage device. Examples of volatile media can include, but are not limited to, dynamic memory. Examples of transmission media can include, but are not limited to, coaxial cables, copper wire, and fiber optics, including the wires that comprise the bus.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

Various forms of computer readable media can be involved in carrying one or more sequences of one or more instructions to the processor for execution. For example, the instructions can initially be carried on the magnetic disk of a remote computer. The remote computer can load the instructions into its dynamic memory and send the instructions over a network connection (e.g., a LAN, a WAN, the internet, a telephone line). A local computer system can receive the data and transmit it to the bus. The bus can carry the data to the memory, from which the processor retrieves and executes the instructions. The instructions received by the memory may optionally be stored on a storage device either before or after execution by the processor.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

In accordance with such a computer system, some embodiments of the technology provided herein further comprise functionalities for collecting, storing, and/or analyzing data (e.g., nucleotide sequence data (e.g., nucleic acid rearrangement junction data)). For example, some embodiments contemplate a system that comprises a processor, a memory, and/or a database for, e.g., storing and executing instructions, analyzing imaging data from a sequencing reaction, performing calculations using the data, transforming the data, and storing the data. It some embodiments, a base-calling algorithm assigns a sequence of bases to the data and associates quality scores to base calls based on a statistical model. In some embodiments, the system is configured to assemble a sequence from multiple sub-sequences, in some instances accounting for overlap and calculating a consensus sequence. In some embodiments, a sequence determined from a sequencing reaction is aligned to a reference sequence or to a scaffold.

Many diagnostics involve determining the presence of, or a nucleotide sequence of, one or more nucleic acids. Thus, in some embodiments, an equation comprising variables representing the presence or sequence properties of multiple nucleic acids produces a value that finds use in making a diagnosis or assessing the presence or qualities of a nucleic acid. As such, in some embodiments this value is presented by a device, e.g., by an indicator related to the result (e.g., an LED, an icon on an LCD, a sound, or the like). In some embodiments, a device stores the value, transmits the value, or uses the value for additional calculations.

Moreover, in some embodiments a processor is configured to control the sequencing reactions and collect the data (e.g., images). In some embodiments, the processor is used to initiate and/or terminate each round of sequencing and data collection relating to a sequencing reaction. Some embodiments comprise a processor configured to analyze the dataset of intensities and/or colors acquired during the sequencing reaction and discern the sequence of the target nucleic acid and/or of its complement.

In some embodiments, a device that comprises a user interface (e.g., a keyboard, buttons, dials, switches, and the like) for receiving user input is used by the processor to direct a measurement. In some embodiments, the device further comprises a data output for transmitting (e.g., by a wired or wireless connection) data to an external destination, e.g., a computer, a display, a network, and/or an external storage medium.

In some embodiments, the technology finds use in providing the sequence of one or more nucleic acids. Accordingly, the technology provided herein finds use in the medical, clinical, and emergency medical fields. In some embodiments a device is used to assay biological samples. In such an assay, the biological sample comprises a nucleic acid and sequencing the nucleic acid is indicative of a state or a property of the sample (e.g., presence and/or identity of one or more nucleic acid rearrangement junctions) and, in some embodiments, the subject from which the sample was taken.

The sequence of output signals provides the sequence of the synthesized DNA and, by the rules of base complementarity, also thus provides the sequence of the template strand.

In various embodiments, the sequencing instrument can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In some embodiments, the nucleic acid is genomic DNA obtained from a subject in need of treatment. In various embodiments, the nucleic acid can include or be derived from a fragment library, a mate pair library, a ChIP fragment, or the like. In particular embodiments, the sequencing instrument can obtain sequence information from a single nucleic acid molecule or from a group of substantially identical nucleic acid molecules.

In various embodiments, the sequencing instrument can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

Some embodiments comprise a system for reconstructing a nucleic acid sequence in accordance with the various embodiments provided herein. The system can include a nucleic acid sequencer, a sample sequence data storage, a reference sequence data storage, and an analytics computing device/server/node. In various embodiments, the analytics computing device/server/node can be a workstation, a mainframe computer, a personal computer, a mobile device, etc.

The nucleic acid sequencer can be configured to analyze (e.g., interrogate) a nucleic acid fragment (e.g., single fragment, mate-pair fragment, paired-end fragment, etc.) utilizing all available varieties of techniques, platforms, or technologies to obtain nucleic acid sequence information, e.g., using sequencing by synthesis, single molecule sequencing, etc. In various embodiments, the nucleic acid sequencer can be in communications with the sample sequence data storage either directly via a data cable (e.g., a serial cable, a direct cable connection, etc.) or bus linkage or, alternatively, through a network connection (e.g., Internet, LAN, WAN, VPN, etc.). In various embodiments, the network connection can be a “hardwired” physical connection. For example, the nucleic acid sequencer can be communicatively connected (via Category 5 (CAT5), fiber optic, or equivalent cabling) to a data server that can be communicatively connected (via CAT5, fiber optic, or equivalent cabling) through the internet and to the sample sequence data storage. In various embodiments, the network connection can be a wireless network connection (e.g., Wi-Fi, WLAN, etc.), for example, utilizing an 802.11b/g or equivalent transmission format. In practice, the network connection utilized is dependent upon the particular requirements of the system. In various embodiments, the sample sequence data storage can be an integrated part of the nucleic acid sequencer.

In various embodiments, the sample sequence data storage can be any database storage device, system, or implementation (e.g., data storage partition, etc.) that is configured to organize and store nucleic acid sequence read data generated by nucleic acid sequencer such that the data can be searched and retrieved manually (e.g., by a database administrator/client operator) or automatically by way of a computer program/application/software script. In various embodiments, the reference data storage can be any database device, storage system, or implementation (e.g., data storage partition, etc.) that is configured to organize and store reference sequences (e.g., whole/partial genome, whole/partial exome, etc.) such that the data can be searched and retrieved manually (e.g., by a database administrator/client operator) or automatically by way of a computer program/application/software script. In various embodiments, sample nucleic acid sequencing read data is stored on the sample sequence data storage and/or the reference data storage in a variety of different data file types/formats, including, but not limited to: *.fasta, *.csfasta, *seq.txt, *qseq.txt, *.fastq, *.sff, *prb.txt, *.sms, *srs, and/or *.qv.

In various embodiments, the sample sequence data storage and the reference data storage are independent standalone devices/systems or implemented on different devices. In various embodiments, the sample sequence data storage and the reference data storage are implemented on the same device/system. In various embodiments, the sample sequence data storage and/or the reference data storage can be implemented on the analytics computing device/server/node.

The analytics computing device/server/node can be in communications with the sample sequence data storage and the reference data storage either directly via a data cable (e.g., serial cable, direct cable connection, etc.) or bus linkage or, alternatively, through a network connection (e.g., Internet, LAN, WAN, VPN, etc.). In various embodiments, the analytics computing device/server/node can host a reference mapping engine, a de novo mapping module, and/or a tertiary analysis engine. In various embodiments, the reference mapping engine can be configured to obtain sample nucleic acid sequence reads from the sample data storage and map them against one or more reference sequences obtained from the reference data storage to assemble the reads into a sequence that is similar but not necessarily identical to the reference sequence using all varieties of reference mapping/alignment techniques and methods. The reassembled sequence can then be further analyzed by one or more optional tertiary analysis engines to identify differences in the genomic sequence (e.g., one or more nucleic acid fusions comprising one or more nucleic acid rearrangement junctions) for the subject relative to a reference sequence. For example, in various embodiments, the tertiary analysis engine can be configured to identify various genomic variants (in the assembled sequence) due to mutations, recombination/crossover, or chromosomal rearrangement.

The optional de novo mapping module can be configured to assemble sample nucleic acid sequence reads from the sample data storage into new and previously unknown sequences.

It should be understood, however, that the various engines and modules hosted on the analytics computing device/server/node can be combined or collapsed into a single engine or module, depending on the requirements of the particular application or system architecture. Moreover, in various embodiments, the analytics computing device/server/node can host additional engines or modules as needed by the particular application or system architecture.

In various embodiments, the mapping and/or tertiary analysis engines are configured to process the nucleic acid and/or reference sequence reads in signal amplitude space. In various embodiments, the mapping and/or tertiary analysis engines are configured to process the nucleic acid and/or reference sequence reads in color space. It should be understood, however, that the mapping and/or tertiary analysis engines disclosed herein can process or analyze nucleic acid sequence data in any schema or format as long as the schema or format can convey the base identity and position of the nucleic acid sequence.

In various embodiments, the sample nucleic acid sequencing read and referenced sequence data can be supplied to the analytics computing device/server/node in a variety of different input data file types/formats, including, but not limited to: *.fasta, *.csfasta, *seq.txt, *qseq.txt, *.fastq, *.sff, *prb.txt, *.sms, *srs and/or *.qv.

In some embodiments, systems comprise computer software configured for designing gRNA (e.g., designing pairs of gRNAs for each nucleic acid rearrangement junction that is targeted). For example, in some embodiments, systems comprise software configured to design gRNA sequences for targeting genomic sequences. See, e.g., Doench (2014) “Rational design of highly active gRNAs for CRISPR-Cas9-mediated gene inactivation.” Nature biotechnology 32: 1262-67; Doench (2016) “Optimized sgRNA design to maximize activity and minimize off-target effects for genetic screens with CRISPR-Cas9.” Nat Biotechnol 34: 184-94; Radzisheuskaya (2016) “Optimizing sgRNA position markedly improves the efficiency of CRISPR/dCas9-mediated transcriptional repression” Nucleic Acids Research 44: e141-e141; Aach (2014) “Flexible algorithm for identifying specific Cas9 targets in genomes” BioRxiv doi: http://dx.doi.org/10.1101/005074 (2014); Bae (2014) “Cas-OFFinder: a fast and versatile algorithm that searches for potential off-target sites of Cas9 RNA-guided endonucleases” Bioinformatics 30: 1473-1475; Heigwer (2014) “E-CRISP: fast CRISPR target site identification” Nat Methods.11: 122-123; Ma (2013) “A guide RNA sequence design platform for the CRISPR/Cas9 system for model organism genomes” Biomed Res Int 2013 (Article ID 270805); Montague (2014) “CHOPCHOP: a CRISPR/Cas9 and TALEN web tool for genome editing” Nucleic Acids Res. 42(W1): W401-W407; Liu (2015) “CRISPR-ERA: a comprehensive design tool for CRISPR-mediated gene editing, repression and activation” Bioinformatics 31: 3676-78; Ran (2015) “In vivo genome editing using Staphylococcus aureus Cas9” Nature 520: 186-91; Wu (2015) “Target specificity of the CRISPR-Cas9 system” Quant Biol 2: 59-70; Xiao (2014) “CasOT: a genome-wide Cas9/gRNA off-target searching tool” Bioinformatics 30: 1180-82; and Haeussler (2016) “Evaluation of off-target and on-target scoring algorithms and integration into the guide RNA selection tool CRISPOR” Genome Biol 17: 1-12, each of which is incorporated herein by reference. In some embodiments, systems comprise use of an online tools, e.g., tools available from Addgene and the ATUM CRISPR gRNA Design tool.

In some embodiments, systems comprise a component for producing gRNA (e.g., for receiving a nucleotide sequence and synthesizing an oligonucleotide comprising the nucleotide sequence). See, e.g., Beaucage (1992) “Advances in the Synthesis of Oligonucleotides by the Phosphoramidite Approach” Tetrahedron 48 (12): 2223; Brown (1993) A brief history of oligonucleotide synthesis. Methods in Molecular Biology (Totowa, N.J., United States), 20 (Protocols for Oligonucleotides and Analogs), 1-17; Reese (2005) “Oligo- and poly-nucleotides: 50 years of chemical synthesis” Organic & Biomolecular Chemistry 3 (21): 3851; Iyer, R. P.; Beaucage, S. L. 7.05. Oligonucleotide synthesis. In: Comprehensive Natural Products Chemistry, Vol. 7: DNA and Aspects of Molecular Biology (Kool, Eric T, ed. (1999), Elsevier, Amsterdam) pp. 105-152; Beaucage (1993) “The functionalization of oligonucleotides via phosphoramidite derivatives” Tetrahedron 49 (10): 1925-1963; Beaucage (1993) “The synthesis of modified oligonucleotides by the phosphoramidite approach and their applications” Tetrahedron 49 (28): 6123-6194; Beaucage, “Oligodeoxyribonucleotides synthesis. Phosphoramidite approach. Methods in Molecular Biology (Totowa, N.J., United States) (1993), 20 (Protocols for Oligonucleotides and Analogs), 33-61; and Reese (2002) “The chemical synthesis of oligo- and poly-nucleotides: a personal commentary”. Tetrahedron. 58 (44): 8893-8920, each of which is incorporated herein by reference. Several commercial suppliers provide custom synthesis of oligonucleotides based on a user-submitted nucleotide sequence.

In some embodiments, systems comprise a means for administration of the gRNA-targeted nuclease and gRNAs. One of ordinary skill in the art can choose the delivery format, delivery vehicle, and formulation for administration to a subject in need of treatment. Delivery formats include, e.g., plasmid DNA, mRNA, or RNP; delivery vehicles include, e.g., viral, non-viral, physical, chemical, and encapsulation. See, e.g., Glass (2018) “Engineering the Delivery System for CRISPR-based Genome Editing” Trends in Biotechnology 36: 173, incorporated herein by reference.

### Uses

The technology finds use in various research, clinical, and medical applications. For example, in some embodiments the technology finds use in treating a subject in need of treatment (e.g., in need of a cancer treatment). In some embodiments, the technology finds use in research for studying cancer in vitro, in vivo, or ex vivo. The technology in some embodiments comprises a method of modifying a cell or organism (e.g., modifying the genome of a cell or organism (e.g., a human cell or a human organism (e.g., a subject in need of a treatment for cancer))). The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish, or shrimp. The cell may also be a plant cell. The plant cell may be of a crop plant such as cassava, corn, sorghum, wheat, or rice. The plant cell may also be of an algae, tree, or vegetable. The modification introduced to the cell by the present technology may be such that the cell and progeny of the cell are altered for improved production of biologic products such as an antibody, starch, alcohol, or other desired cellular output. The modification introduced to the cell by the present technology may be such that the cell and progeny of the cell include an alteration that changes the biologic product produced.

The technology may comprise use of one or more different vectors. In some embodiments of the technology, the gRNA-targeted nuclease (e.g., a dCas9-Fok1 fusion) is codon optimized for expression by the desired cell type, preferentially a eukaryotic cell, preferably a mammalian cell or a human cell.

In some embodiments, packaging cells are used to form virus particles that are capable of infecting a host cell. Such cells include 293 cells, which package adenovirus, and psi2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, hut lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, one or more vectors described herein are used to produce a non-human transgenic animal or transgenic plant. In some embodiments, the transgenic animal is a mammal, such as a mouse, rat, or rabbit. Methods for producing transgenic animals and plants are known in the art, and generally begin with a method of cell transfection, such as described herein. In another embodiment, a fluid delivery device with an array of needles (see, e.g., US Patent Publication No. 20110230839 assigned to the Fred Hutchinson Cancer Research Center) may be contemplated for delivery of a gRNA-targeted nuclease (e.g., a dCas9-Fok1 fusion) to solid tissue. A device of US Patent Publication No. 20110230839, incorporated herein by reference, for delivery of a fluid to a solid tissue may comprise a plurality of needles arranged in an array; a plurality of reservoirs, each in fluid communication with a respective one of the plurality of needles; and a plurality of actuators operatively coupled to respective ones of the plurality of reservoirs and configured to control a fluid pressure within the reservoir.

In some embodiments, the technology provides for methods of modifying a target polynucleotide in a eukaryotic cell. In some embodiments, the method comprises allowing a nucleic acid-targeting complex to bind to the target polynucleotide to effect cleavage of said target polynucleotide thereby modifying the target polynucleotide, wherein the nucleic acid-targeting complex comprises a gRNA-targeted nuclease (e.g., a dCas9-Fok1 fusion) complexed with a guide RNA hybridized to a target sequence within said target polynucleotide.

### EXAMPLES

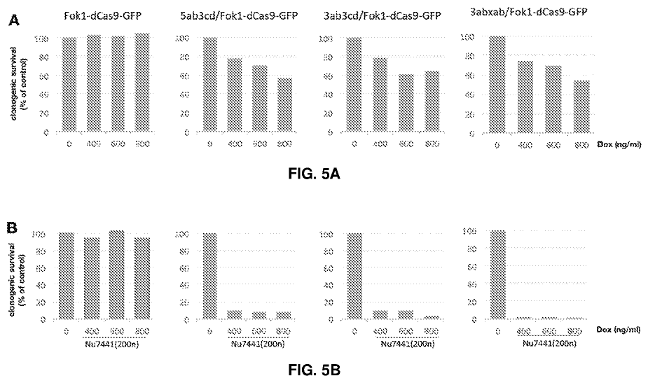

During the development of embodiments of the technology provided herein, experiments were conducted to evaluate use of a dCas9-Fok1 fusion protein and to produce a dCas9-Fok1 fusion protein further comprising a GFP domain to provide a tool to detect expression in cells. Furthermore, the dCas9-Fok1-GFP fusion and the gRNA genes were placed under the regulation of a tet-inducible promoter for performing experiments to establish the cells in culture or as xenografts and then induce expression by addition of doxycycline (Dox). The HCT116 colorectal cancer cell line was used for experiments because it is easily transfected and its genome has been sequenced and all nucleic acid rearrangement junctions are known. After evaluating 10 nucleic acid rearrangement junction sequences in these cells, four nucleic acid rearrangement junctions were identified that comprised favorable locations of PAM sequences for dCas9 positioning. A pair of gRNAs was designed for each of the four nucleic acid rearrangement junctions and the gRNA coding sequences for each pair were cloned into vectors expressing dCas9-Fok1-GFP. The vectors were then used to transfect HCT116 cells to generate four cell lines each providing dox-inducible expression of dCas9-Fok1-GFP and a pair of gRNAs. Addition of increasing concentrations of Dox resulted in a 40-50% reduction in clonogenic survival in all the three cell lines expressing dCas9-Fok1-GFP and two pairs of gRNAs (FIG. 5A). However, no such loss of clonogenicity was observed in the cell line expressing dCas9-Fok1-GFP without gRNAs. The data indicated that the technology described herein for targeting nucleic acid rearrangement junctions in the HCT116 cells produces loss of cancer cell fitness (e.g., survival). More impressively, when Dox-induction of dCas9-Fok1-GFP and gRNAs was combined with adding the DNA-PK inhibitor Nu7441, cell survival decreased below 10% with no effect observed in the control cells only expressing dCas9-Fok1-GFP (FIG. 5B). DSBs are induced by the dimerization of Fok1 endonuclease at the targeted nucleic acid rearrangement junction and the cells become less able to survive these breaks when the repair of these DSBs is inhibited by the DNA-PK inhibitor Nu7441.

### Identification of CRJs in HCT116 Cells

Experiments were conducted using the colon cancer cell line HCT116. This cell line has been sequenced and all CRJs present in HCT113 cells are known (see, e.g., the Cosmic database provided by the Sanger Center; Tate et al. (2019) “COSMIC: the Catalogue Of Somatic Mutations In Cancer” Nucleic Acids Research 47(D1): D941-D947, incorporated herein by reference). Four different CRJs were selected for targeting (out of >100 possible CRJs) based criteria including favorable positioning of PAM sequences on either side of the CRJs (see, e.g., FIG. 1 (generally) and FIG. 3 (specifically)). Two CRJs were located on chromosome 3, one on chromosome 5, and one on the X chromosome. Guide RNA (gRNA) sequences were designed and synthesized. The sequences of the guide RNAs targeting chromosomes 3, 5, and X are provided in Table 1.

TABLE 1
gRNAs targeting CRJs in HCT116 cells

| | | SEQ |
|---|---|---|
| | | ID |
| Name | sequence (5′ to 3′) | NO: |
| 3aFw | CACCGGGAATGCGATTTACAAGCTT | 4 |
| 3aRev | AAACAAGCTTGTAAATCGCATTCCC | 5 |
| 3bFw | CACCGCTAGCAATTCTTAGCTGTCA | 6 |
| 3bRev | AAACTGACAGCTAAGAATTGCTAGC | 7 |
| 3cFw | CACCGTGGCAACCATTAGGAATAAC | 8 |
| 3cRev | AAACGTTATTCCTAATGGTTGCCAC | 9 |
| 3dFw | CACCGAAAGTGGTTCTGTCTTAAA | 10 |
| 3dRev | AAACTTTAAGACAGAACCACTTT | 11 |
| 5aFw | CACCGAAAATCTCTGTCTTCAAAAG | 12 |
| 5aRev | AAACCTTTTGAAGACAGAGATTTTC | 13 |
| 5bFw | CACCGTGCATGCTAAAGTGTGTGAA | 14 |
| 5bRev | AAACTTCACACACTTTAGCATGCAC | 15 |
| xaFw | CACCGTGAATATATTTTTCTCAGCC | 16 |
| xaRev | AAACGGCTGAGAAAAATATATTCAC | 17 |
| xbFw | CACCGGCATTCTACCACCTGGTCTT | 18 |
| xbRev | AAACAAGACCAGGTGGTAGAATGCC | 19 |

Cloning of dCas9-Fok1-GFP

A GFP domain and a dox-inducible regulatory element (TRE3G) were cloned into a vector comprising a dCas9-Fok1 fusion. The pX330A_dCas9-Fok1-1x4 (see, e.g., Tsai et al. (2014) “Dimeric CRISPR RNA-guided Fok1 nucleases for highly specific genome editing” Nature biotechnology 32, 569-576, incorporated herein by reference), pSLQ1658-dCas9-EGFP (see, e.g., Chen et al. (2013) “Dynamic Imaging of Genomic Loci in Living Human Cells by an Optimized CRISPR/Cas System” Cell 155(7): 1479-91, incorporated herein by reference), and single gRNA expressing plasmid vectors were obtained from Addgene (see, e.g., Nakagawa (2015) “Production of knockout mice by DNA microinjection of various CRISPR/Cas9 vectors into freeze-thawed fertilized oocytes” BMC Biotechnol 15: 33; and Sakuma (2014) “Multiplex genome engineering in human cells using all-in-one CRISPR/Cas9 vector system” Sci Rep 4: 5400, each of which is incorporated herein by reference.

An amplicon comprising the enhanced green fluorescent protein (EGFP) gene and the puromycin resistance gene (PuroR) was produced from the pSLQ1658-dCas9-EGFP vector using polymerase chain reaction. The PCR strategy introduced a restriction enzyme recognition site for PsiI at the C-terminus of the PCR fragment. The pX330A_dCas9-Fok1-1x4 plasmid was digested with EcoRV and PsiI and the fragment comprising dCas9-Fok1 was gel purified. The EGFP-PuroR amplicon was also digested with EcoRV and PsiI and gel purified. After ligating the fragment encoding dCas9-Fok1 with the fragment encoding EGFP and PuroR using T4 ligase, the ligation product (pX330A_dCas9-Fok1-GFP) was transformed into Mach1 competent cells. Colonies of pX330A_dCas9-Fok1-GFP were picked and validated by DNA sequencing.

Cloning of dCas9-Fok1-GFP with Dox-Inducible Regulatory Element (TRE3G)

The TRE3G promoter was cloned into the pX330A_dCas9-Fok1-GFP vector. First, the CMV promoter in the pX330A_dCas9-Fok1-GFP vector was released by digesting the pX330A_dCas9-Fok1-GFP vector with KpnI and AgeI double digestion. The Dox-inducible regulatory element (TRE3G) promoter was obtained from the pLVX-TRE3G vector (Clontech Laboratories) using PCR primers flanking the TRE3G promoter and designed to have a KpnI recognition site on the forward primer tail and an AgeI recognition site on the reverse primer tail. The TRE3G fragment was amplified and gel purified. The pTRE3G-dCas9-Fok1-GFP construct was obtained by assembling the two fragments using Gibson assembly (Gibson et al. (2009) “Enzymatic assembly of DNA molecules up to several hundred kilobases” Nature Methods 6: 343-345, incorporated herein by reference) and the recombinant products were validated by DNA sequence analysis.

Cloning of Pairs of CRJ-Targeting gRNAs

Three different gRNA pairs were cloned into individual vectors. CRJs located on chromosomes 3, 5, and X were selected for targeting by complementary gRNA (see above). Pairs of gRNA cassettes named “3ab”, “3cd”, “5ab”, and “xab” were designed using the ATUM CRISPR gRNA design tool. The gRNA design and targeting strategy incorporated an offset of 11 to 18 bases for gRNA pairs to provide high efficiency targeting (see, e.g., Tsai, supra). Annealed 3a and 5a gRNA oligonucleotides were cloned into the pX330A_Fok1-1x4 vector, annealed 3b and 5b gRNA oligonucleotides were cloned into the pX330S-2 vector, annealed 3c and xa gRNA oligonucleotides were cloned into the pX330S-3 vector, and annealed 3d and Xb gRNA oligonucleotides were cloned into the pX330S-4 vectors using PpiI digestion and T4 ligation. Next, golden gate assembly (see, e.g., Engler et al. (2008) PLoS ONE 3: e3647; and Engler et al. (2009) PLoS ONE 4: e5553, each of which is incorporated herein by reference) was employed using BsaI digestion and ligation using 25 cycles of 37° C. for 5 minutes and 16° C. for 19 minutes. The assembled vectors were validated by DNA sequencing.

Immunofluorescence Staining of gH2AX

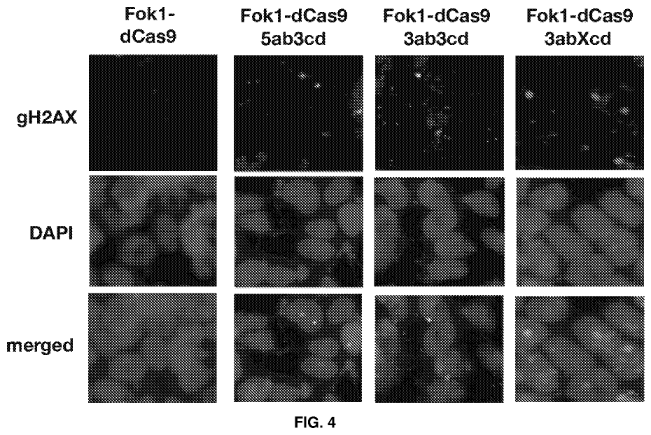

HCT116 cells were then transfected with the four vectors and cells expressing each vector were selected with puromycin to produce four cell lines with dox-inducible expression of the different vectors. dCas9-Fok1-GFP, 3ab3cd/Fok1-dcas9-GFP, 5ab3cd/Fok1-GFP, and 3abxab/dCas9-GFP HCT116 cell lines were cultured in DMEM containing 10% fetal bovine serum (FBS) on chamber slides coated with 0.01% poly-L-lysine. Fok1-GFP expression was induced by adding doxycycline (600 ng/ml) and 200 nM Nu7441, an inhibitor of the DNA-dependent protein kinase (DNA-PK) complex, for 24 hours. Cells were fixed with 4% paraformaldehyde and evaluated by immunofluorescence using a mouse monoclonal antibody against gH2AX at a dilution of 1:300, followed by Cy3-conjugated ani-mouse secondary antibody at a 1:500 dilution. Following multiple stringent washes, images were recorded using a fluorescence microscope. gH2AX foci were only formed in cells expressing both the dCas9-Fok1-GFP and CRJ-targeting gRNA pairs (see FIG. 4).

### Clonogenic Survival

HCT116 cells with stable expression of dCas9-Fok1-GFP, 3ab3cd/Fok1-dcas9-GFP, 5ab3cd/Fok1-GFP, and 3abxab/dCas9-GFP were maintained in DMEM containing 10% FBS. Cells were dissociated by trypsin/EDTA and then washed once with DMEM containing 10% FBS and filtered through a cell strainer to obtain a single cell suspension. 500 cells were seeded in 60-mm falcon dishes. At 2 hours after seeding, cells were treated with Doxycycline at doses of 0, 400, 600 and 800 ng/ml in the absence or presence of the DNA-PK inhibitor Nu7441 at a final concentration of 200 nM for 12 days (FIG. 5A and FIG. 5B). Cells were fixed with 0.5% paraformaldehyde and stained with crystal violet. Clonogenic survival colonies with 50 cells or more were counted. Data from three independent experiments indicated that induction of dCas9-Fok1-GFP with Dox in HCT116 cells did not result in toxicity (FIG. 5A, first plot from the left). However, induction of dCas9-Fok1-GFP together with targeting gRNA pairs resulted in a reduction of clonogenc survival by 20-40% (FIG. 5A, second, third, and fourth plots from the left). Adding the DNA-PK inhibitor Nu7441 to cells expressing dCas9-Fok1-GFP alone did not affect clonogenicity of the cells (FIG. 5B, first plot from the left). However, addition of Nu7441 to cells expressing both dCas9-Fok1-GFP and two pairs of CRJ-targeting gRNA oligonucleotides resulted in a loss of clonogenicity (FIG. 5B, second, third, and fourth plots from the left).

dCas9-Fok1 Targeting Induces DSBs

Experiments conducted during the development of embodiments of the technology provided herein indicated that Dox-induced expression of dCas9-Fok1 reduces fitness of HCT116 cells when targeted to sites comprising CRJs using gRNA pairs (FIG. 5A and FIG. 5B). Further experiments are conducted to confirm that DSBs are formed by the dimerization of the Fok1 endonuclease at the CRJs. Accordingly, experiments are conducted to assess the induction of γH2AX (histone H2AX phosphorylated on serine 139) using immunohisto-chemistry and γH2AX ChIP-PCR. Phosphorylation of the γH2AX variant of histone H2AX occurs rapidly in chromatin adjacent to DSBs after their formation and therefore provides a signal indicating the presence of DSBs. HCT116 cells are grown on coverslips. Expression of dCas9-Fok1-GFP and gRNAs is induced using Dox and Nu7441 is added to prohibit repair of DSBs produced at the targeted CRJs. After different incubation times, cells are fixed and immunohistochemistry performed using anti-γH2AX antibodies and fluorescent secondary antibodies (e.g., comprising a red fluorescent label). Images of the fixed and stained cells are captured using a florescence microscope. Cells expressing the dCas9-Fok1-GFP appear green (from GFP fluorescence) and γH2AX foci appear red (e.g., from fluorescence of the secondary antibody) and indicate the presence of DSBs. Experiments also comprise use of γH2AX ChIP-PCR at different times following Dox-induction of the dCas9-Fok1-GFP and gRNAs in the HCT116 cells to determine whether γH2AX is induced preferentially at sites of targeted CRJs (e.g., indicating the presence of DSBs). Data indicating two γH2AX foci in HCT116 cells after addition of Dox and Nu7441 indicate specific targeting and activation of dCas9-Fok1-GFP at cancer-specific CRJs (e.g., because the cells express two pairs of CRJ-targeting gRNAs) to produce DSBs at the CRJ sites. In contrast, no γH2AX foci are observed in the HCT116 cells expressing dCas9-Fok1-GFP without the targeting gRNAs. Furthermore, DNA sequences adjacent to the targeted CRJs are preferentially isolated by ChIP-PCR, thus producing a stronger PCR signal than in cells without CRJ-targeting gRNAs.

Studies have shown that unstressed HCT116 cells do not present with γH2AX foci (see, e.g., Mirzayans et al. (2015) “Spontaneous gammaH2AX Foci in Human Solid Tumor-Derived Cell Lines in Relation to p21WAF1 and WIP1 Expression” Int J Mol Sci 16: 11609-11628, incorporated herein by reference). Thus, the HCT116 cells do not show “spontaneous” γH2AX foci that could complicate determining whether the induction of the Fok1 endonuclease results in two additional γH2AX foci. Nevertheless, the γH2AX-ChIP-PCR specifically amplifies targeted CRJs and therefore provides detection of DSBs produced by the technology provided herein in a background of random spontaneous DSB breaks because random spontaneous DSBs will not occur repeatedly at the same site. Further, ChIP-PCR using anti-GFP antibodies is used to detect preferential binding of the dCas9 complex to CRJs.

### Targeting Tumors In Vivo

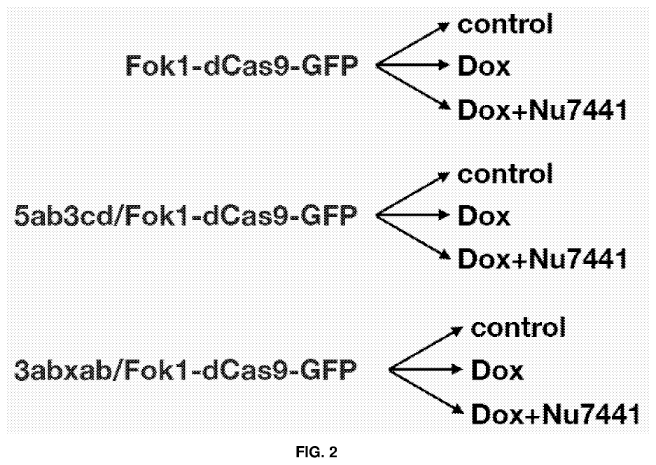

Experiments are conducted in which three of the HCT116 cell lines with dox-inducible expression of dCas9-Fok1-GFP with and without CRJ-targeting gRNAs are used to form xenografts in the flank of mice. In these experiments, one million HCT116 cells in a 100 μl Matrigel suspension are injected s.c. into the hind leg of the mice (a total of 54 NOD/SCID or athymic BALB/C, 10-12 weeks old). When tumors reach sizes of 50-150 mm³, each group is split into three groups (see, e.g., FIG. 2) where they receive ether regular water, water with Dox, or water with Dox and Nu7441. Tumor measurements are taken daily and mouse weights are recorded 3 times weekly. As the control tumors reach the predetermined tumor size limit, animals are euthanized. At this time, the tumors are removed, weighed, and documented (e.g., using digital imaging) and used for downstream analyses. Downstream analyses include measuring expression of dCas9-Fok1-GFP (green fluorescence) and γH2AX ChIP-PCR with primers for sequences near the CRJ and sequences far away as a control.

Similar to the data described herein evaluating clonogenic survival of HCT116 cells in culture, activation of the Fok1 endonuclease by gRNA targeting in vivo induces DSBs at CRJs, which slows tumor growth. Suppression of tumor growth is augmented by the addition of Nu7441. Expression of GFP is observed in the tumors of animals receiving Dox in their drinking water and γH2AX is enriched at the targeted CRJs. The amount of Dox sufficient to activate expression of the dCas9-Fok1-GFP in vivo is assessed by measuring the expression of GFP in the tumor and providing additional administration of Dox and/or Nu7441 (e.g., through intravenous injections) if needed.

### Targeting CRJs in Human Bladder Cancer Cells In Vitro and In Vivo

As described herein, experiments were conducted during the development of embodiments of the technology described herein and data were collected that indicated that Fok1-dCas9 and gRNAs can be used to precisely induce DSBs at CRJs in colon cancer cells, thus leading to significant loss of fitness of cancer cells.

During the development of embodiments of the technology provided herein, the same or similar approach was used to kill bladder cancer cells. Specifically, experiments were conducted to use the technology to target bladder cancer cells in vitro and in orthotopic bladder cancer models in vivo.

First, CRJs were identified in bladder cancer cells (see, e.g., FIG. 6A). Using sequence information from the CRJs identified in bladder cancer cells, gRNA were designed and UMUC-3 (ATCC Accession CRL-1749) cell lines were produced comprising doxycycline-inducible expression of Fok1-dCas9 and the CRJ-targeting gRNAs. gRNA sequences are provided in Table 2.

+0 TABLE 2
gRNAs targeting CRJs in UMUC-3 cells

| | | SEQ |
|---|---|---|
| | | ID |
| Name | sequence (5′ to 3′) | NO: |
| UC3-19a | CAAGGTGGCGCAGTTTTCCC | 20 |
| UC3-19a′ | GGGAAAACTGCGCCACCTTG | 21 |
| UC3-19b | TTATGTAAAAATGTGTGGGC | 22 |
| UC3-19b′ | GCCCACACATTTTTACATAA | 23 |
| UC3-2a | CATCCCTATATCTTAACTCA | 24 |
| UC3-2a′ | TGAGTTAAGATATAGGGATG | 25 |
| UC3-2b | GTACCTCTTCACATTGTTGC | 26 |
| UC3-2b′ | GCAACAATGTGAAGAGGTAC | 27 |
| UC3-4a | ACAGCCCAGATACCATGTTG | 28 |
| UC3-4a′ | CAACATGGTATCTGGGCTGT | 29 |
| UC3-4b | TTATTGCTATATGTAACTGA | 30 |
| UC3-4b′ | TCAGTTACATATAGCAATAA | 31 |
| UC3-7a | CATTATTGGGATATCCTTTA | 32 |
| UC3-7a′ | TAAAGGATATCCCAATAATG | 33 |
| UC3-7b | AAGAGATTTCAGGCTGGGCG | 34 |
| UC3-7b′ | CGCCCAGCCTGAAATCTCTT | 35 |

Six UMUC-3 bladder cancer sublines were generated—Two control cell lines were produced, one expressing no gRNAs and one expressing 2 pairs of non-targeting gRNAs; four experimental cell lines were produced expressing Fok1-dCas9-GFP and two pairs of CRJ-targeting gRNAs under the regulation of doxycycline (see, e.g., FIG. 6B). CRJ-specific DSBs were induced in the cells (e.g., using doxycycline) and toxicity was monitored to determine if interfering with DSB repair leads to increased killing of bladder cancer cells (see, e.g., FIG. 6C, top row). Similar experiments were conducted to determine if interfering with DSB repair and treating with a DNA-PK inhibitor produces more killing of bladder cancer cells (see, e.g., FIG. 6C, bottom row).

The data collected from experiments using the UMUC-3 bladder cancer cells indicated that the technology described herein successfully targeted CRJs in the bladder cancer cells. Induction of the Fok1-dCas9 and CRJ-targeting gRNA produced a significant loss of cell fitness (FIG. 6C). In contrast, control cells expressing nontargeting gRNAs showed no loss in cell fitness (FIG. 6C). Further, the data indicated that the cells in which Fok1 and gRNA were induced to produce DSB had decreased survival and that Nu7441 augmented this effect. Taken together, these data indicate that the technology provided herein successfully induced DSBs specifically at sites of CRJs in a plurality of different tumor cell types.

As described above, experiments conducted with colon and bladder cancer cells indicated that expressing pairs of CRJ-targeting gRNAs induced DSBs that decreased cell fitness. Accordingly, further experiments are conducted to grow these cell lines as orthotopic xenografts in NOD-scid mice and to monitor tumor growth (e.g., using bioluminescence) in vivo as the CRISPR reagents are induced by doxycycline (e.g., provided in food and/or water). UMUC-3 cells are grown as orthotopic tumors in NOD-scid mice, animals are given doxycycline to induce Fok1-dCas9 and the two pairs of gRNAs in the cancer cells, and the effects on tumor growth are assessed by bioluminescence. Experiments use a control cell line expressing two pairs of gRNA that target CRJs in HCT116 cells (see, e.g., FIG. 3), but that do not target CRJs in UMUC-3 cells (FIG. 6C). The other cell line expresses CRJ-targeting gRNAs following doxycycline exposure. Experiments are conducted with or without a DNA-PK inhibitor (e.g., Nu7441) and with and without doxycycline in the drinking water and/or in the food. 10 animals are used per group with 8 groups for a total of 80 animals (FIG. 7).

In these in vivo experiments, induction of apoptosis is evaluated (e.g., by evaluating fractional (subG1) DNA content, caspase 3 activation, and/or PARP1 cleavage) and clonogenic survival is evaluated. Further, experiments are conducted in which DDR signaling is inhibited (e.g., using the ATM inhibitor Ku55933 (e.g., 10 μM) available from Merck-Millipore and/or ATR inhibitor VE-821 (e.g., 10 μM) available from Selleck) and/or cell cycle checkpoints are inhibited (e.g., using the WEE1 inhibitor MK-1775 (e.g., 3 mM) available from Sigma) to augment the cell killing effects of the CRJ targeting technology described herein. See, e.g., Weber (2015) “ATM and ATR as therapeutic targets in cancer” Pharmacology & Therapeutics 149: 124-38, incorporated herein by reference.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method of treating a subject having cancer or in need of a cancer treatment, the method comprising:
a) identifying a nucleic acid rearrangement junction in nucleotide sequence data obtained from a sample from said subject; and
b) contacting a nucleic acid comprising said nucleic acid rearrangement junction with a gRNA-guided nuclease, a first gRNA, and a second gRNA.

2. The method of claim 1 further comprising obtaining the sample from said subject; and producing or having produced said nucleotide sequence data from a nucleic acid obtained from the sample.

3. The method of claim 1 wherein said first gRNA is complementary to a first target sequence of said nucleic acid comprising said nucleic acid rearrangement junction and said second gRNA is complementary to a second target sequence of said nucleic acid comprising said nucleic acid rearrangement junction.

4. The method of claim 3 wherein said first target sequence and said second target sequence flank said nucleic acid rearrangement junction.

5. The method of claim 3 wherein said first target sequence comprises said nucleic acid rearrangement junction and said second target sequence is adjacent to said nucleic acid rearrangement junction.

6-8. (canceled)

9. The method of claim 1 wherein said gRNA-guided nuclease is a first gRNA-guided nuclease and further comprising contacting said nucleic acid comprising said nucleic acid rearrangement junction with a second gRNA-guided nuclease.

10. The method of claim 9 wherein said first gRNA-guided nuclease and said second gRNA-guided nuclease form a dimer.

11. The method of claim 10 wherein said dimer produces a double stranded break in said nucleic acid.

12. The method of claim 1 wherein said gRNA-guided nuclease is a dCas9-Fok1 protein.

13. The method of claim 9 wherein said first gRNA-guided nuclease is a dCas9-Fok1 protein and said second gRNA-guided nuclease is a dCas9-Fok1 protein.

14. The method of claim 1 further comprising administering an effective amount of an inhibitor of double stranded break repair to said subject.

15-16. (canceled)

17. The method of claim 1 wherein said sample comprises a cancer cell.

18. The method of claim 1 wherein said sample is obtained from a biopsy sample from said subject.

19-20. (canceled)

21. The method of claim 1 further comprising analyzing said nucleotide sequence data and designing said first gRNA and said second gRNA to target said nucleic acid comprising said nucleic acid rearrangement junction.

22. The method of claim 1 further comprising synthesizing or having synthesized said first gRNA and said second gRNA.

23. The method of claim 1 further comprising administering said gRNA-guided nuclease or a nucleic acid encoding said gRNA-guided nuclease, said first gRNA, and said second gRNA to said subject.

23. The method of claim 1 comprising identifying a plurality of nucleic acid rearrangement junctions in said nucleotide sequence data.

24. The method of claim 23 comprising designing a specific gRNA pair targeting each of said nucleic acid rearrangement junctions.

25. The method of claim 24 comprising contacting each of a plurality of nucleic acids, wherein each nucleic acid comprises a nucleic acid rearrangement junction, with a specific gRNA pair and a gRNA-guided nuclease.

26. The method of claim 23 wherein said plurality of nucleic acid rearrangement junctions comprises 1-10, 1-20, 1-50, or 1-100 nucleic acid rearrangement junctions.
